(19) United States
(12) Patent Application Publication
(10) Pub. No.: US 2014/0322705 A1
(43) Pub. Date: Oct. 30, 2014

(54) Cancer diagnostic and cancer therapeutic

(71) Applicants: Michael P. Lisanti, Withington (GB); Federica Sotgia, Withington (GB)

(72) Inventors: Michael P. Lisanti, Withington (GB); Federica Sotgia, Withington (GB)

(73) Assignee: Thomas Jefferson University, Philadelphia, PA (US)

(21) Appl. No.: 13/817,687

(22) PCT Filed: Aug. 19, 2011
(86) PCT No.: PCT/US11/48467

§ 371 Date: May 16, 2014

Related U.S. Application Data
(60) Provisional application No. 61/375,364, filed on Aug. 20, 2010.
(60) Provisional application No. 61/375,369, filed on Aug. 20, 2010.

(51) Int. Cl. C12Q 1/68; G01N 33/574

(52) U.S. Cl. CPC C12Q 1/6869; G01N 33/57496; C12Q 1/6844; G01N 2800/52
USPC 435/6.11; 435/6.1; 435/7.1; 435/6.12

(57) ABSTRACT

This invention relates to methods and kits for making a prognosis of disease course in a neoplastic disease patient by determining the level, expression and/or activity of a biomarker.

### BACKGROUND OF THE INVENTION

1. Field of Invention

This invention relates to methods and kits for making a prognosis of disease course in a neoplastic disease patient by determining the level, expression and/or activity of a biomarker.

2. Description of Related Art

It is now well-recognized that the tumor micro-environment plays a primary role in determining tumor progression and metastasis in many different types of epithelial cancers. In this regard, “activated or myofibroblastic” cancer-associated fibroblast have emerged as one of the most prominent cell types in the tumor stroma that may determine clinical outcome in breast and prostate cancers. We have recently identified a loss of stromal Cav-1 in the tumor associated fibroblast compartment as a critical event in the progression of human breast cancers (Mercier I, Casimiro M C, Wang C, Rosenberg A L, Quong J, Allen K G, Danilo C, Sotgia F, Bonnucelli G, Jasmin J F, Xu H, Bosco E, Aronow B, Witkiewicz A, Pestell R G, Knudsen E S, Lisanti M P. Human Breast Cancer-Associated Fibroblasts (CAFs) Show Caveolin-1 Down-regulation and RB Tumor Suppressor Functional Inactivation: Implications for the Response to Hormonal Therapy. Cancer Biol Ther 2008; 7:1212-25; Sotgia F, Del Galdo F, Casimiro M C, Bonuccelli G, Mercier I, Whitaker-Menezes D, Daumer K M, Zhou J, Wang C, Katiyar S, Xu H, Bosco E, Quong A A, Aronow B, Witkiewicz A K, Minetti C, Frank P G, Jimenez S A, Knudsen E S, Pestell R G, Lisanti M P. Caveolin-1−/− null mammary stromal fibroblasts share characteristics with human breast cancer-associated fibroblasts. Am J Pathol 2009; 174:746-61; Witkiewicz A K, Dasgupta A, Sotgia F, Mercier I, Pestell R G, Sabel M, Kleer C G, Brody J R, Lisanti M P. An Absence of Stromal Caveolin-1 Expression Predicts Early Tumor Recurrence and Poor Clinical Outcome in Human Breast Cancers. Am J Pathol 2009; 174:2023-34.). More specifically, a loss of stromal Cav-1 is associated with early tumor recurrence, lymph node metastasis, and tamoxifen-resistance, resulting in poor clinical outcome (Witkiewicz A K, Dasgupta A, Sotgia F, Mercier I, Pestell R G, Sabel M, Kleer C G, Brody J R, Lisanti M P. An Absence of Stromal Caveolin-1 Expression Predicts Early Tumor Recurrence and Poor Clinical Outcome in Human Breast Cancers. Am J Pathol 2009; 174:2023-34). Similar results were obtained with DCIS (10) and prostate cancer patients (Di Vizio D, Morello M, Sotgia F, Pestell R G, Freeman M R, Lisanti M P. An Absence of Stromal Caveolin-1 is Associated with Advanced Prostate Cancer, Metastatic Disease and Epithelial Akt Activation. Cell Cycle 2009; 8:2420-4.), indicating that a loss of stromal Cav-1 in cancer-associated fibroblasts is tightly associated with tumor progression and metastasis. These findings have now been replicated in several independent patient cohorts (Witkiewicz A K, Casimiro M C, Dasgupta A, Mercier I, Wang C, Bonuccelli G, Jasmin J F, Frank P G, Pestell R G, Kleer C G, Sotgia F, Lisanti M P. Towards a new “stromal-based” classification system for human breast cancer prognosis and therapy. Cell Cycle 2009; 8:1654-8; Sloan E K, Ciocca D, Pouliot N, Natoli A, Restall C, Henderson M, Fanelli M, Cuello-Carrion F, Gago F. Anderson R. Stromal Cell Expression of Caveolin-1 Predicts Outcome in Breast Cancer. Am J Pathol 2009; 174:2035-43.), and also extended to other more lethal forms of breast cancer, such as the triple-negative and basal-like breast cancer sub-types (Witkiewicz A K, Dasgupta A, Sammons S, Er O, Potoczek M B, Guiles F. Sotgia F, Brody J R, Mitchell E P, Lisanti M P. Loss of stromal caveolin-1 expression predicts poor clinical outcome in triple negative and basal-like breast cancers. Cancer Biol Ther 2010; 10: In Press). For example, in triple-negative breast cancers, patients with high stromal Cav-1 have a 75.5% survival rate at 12 years, while patients with an absence of stromal Cav-1 have a survival rate of less than 10% at 5 years post-diagnosis (Wikiewicz, 2010). Thus, the inventors have dissected the molecular basis of this phenomenon, to design better therapeutics targeting this high-risk patient population.

To mechanistically understand the lethality of a Cav-1-deficient stromal microenvironment, Cav-1 (−/−) null mice was used as a model system (Pavlides S, Whitaker-Menezes D, Castello-Cros R, Flomenberg N, Witkiewicz A K, Frank P G, Casimiro M C, Wang C, Fortina P, Addya S, Pestell R G, Martinez-Outschoorn U E, Sotgia F, Lisanti M P. The reverse Warburg effect: aerobic glycolysis in cancer associated fibroblasts and the tumor stroma. Cell Cycle 2009; 8:3984-4001). Using this approach, bone-morrow stromal cells were isolated (thought to be the precursors of cancer-associated fibroblasts (Mishra P J, Humeniuk R, Medina D J, Alexe G, Mesirov J P, Ganesan S, Glod J W, Banerjee D. Carcinoma-associated fibroblast-like differentiation of human mesenchymal stem cells. Cancer Res 2008; 68:4331-9), and subjected them to unbiased proteomics and genome-wide transcriptional profiling (Pavlides, 2009). As a result, via our proteomics analysis, it was observed that Cav-1 (−/−) null stromal cells show the upregulation of i) 8 myo-fibroblast markers (including vimentin, calponin, and collagen 1), 8 glycolytic enzymes (such as pyruvate kinase (PKM2) and lactate dehydrogenase (LDHA), and 2 markers of oxidative stress (namely catalase and peroxiredoxin) (Bissell M J, Radisky D. Putting tumours in context. Nat Rev Cancer 2001; 1:46-54) (Wikiewicz, 2010). These results were also independently supported by our data from transcriptional profiling. An informatics analysis of these findings suggested that a loss of stromal Cav-1 results in oxidative stress, driving aerobic glycolysis (a.k.a., the Warburg effect) in cancer-associated fibroblasts (Pavlides S, Tsirigos A, Vera I, Flomenberg N, Frank P G, Casimiro M C, Wang C, Fortina P, Addya S, Pestell R G, Rigoutsos I, Martinez-Outschoorn U E, Sotgia F, Lisanti M P. Loss of Stromal Caveolin-1 Leads to Oxidative Stress, Mimics Hypoxia, and Drives Inflammation in the Tumor Microenvironment, Conferring the “Reverse Warburg Effect”: A Transcriptional Informatics Analysis with Validation. Cell Cycle 2010; In Press; Pavlides S, Tsirigos A, Vera I, Flomenberg N, Frank P G, Casimiro M C, Wang C, Pestell R G, Martinez-Outschoorn U E, Howell A, Sotgia F, Lisanti M P. Transcriptional evidence for the “Reverse Warburg Effect” in human breast cancer tumor stroma and metastasis: similarities with oxidative stress, inflammation, Alzheimer's disease, and “Neuron-Glia Metabolic Coupling”. Aging (Albany N.Y.) 2010; 2:185-99.). This would then provide a feed-forward mechanism by which Cav-1-deficient cancer-associated fibroblasts could literally “feed” adjacent cancer cells, by providing lactate and pyruvate in a paracrine fashion (Wikiewicz, 2010). The inventors have termed this novel idea the “Reverse Warburg Effect”, as the original Warburg effect was originally thought to occur only in epithelial cancer cells and not cancer-associated fibroblasts (Wikiewicz, 2010).

The inventors have now performed an unbiased metabolomics analysis on the mammary fad pads derived from Cav-1 (−/−) null mice to validate the existence of the “Reverse Warburg Effect”. However, what was observed was far more complex and extensive, and was characteristic of a profound catabolic phenotype. The results are consistent with oxidative stress, mitochondrial dysfunction, and autophagy/mitophagy—which would also induce aerobic glycolysis in the tumor stroma (the “Reverse Warburg Effect”).

Interestingly, autophagy, mitophagy, and aerobic glycolysis are all induced by oxidative stress and are all controlled by the same key transcription factor, namely hypoxia-inducible factor-1-alpha (HIF1-alpha). In this regard, the inventors directly show that a loss of stromal Cav-1 leads to the up-regulation of miR-31, which is a known activator of HIF1alpha transcriptional activity (Liu C J, Tsai M M, Hung P S, Kao S Y, Liu T Y, Wu K J, Chiou S H, Lin S C, Chang K W. miR-31 ablates expression of the HIF regulatory factor FIH to activate the HIF pathway in head and neck carcinoma. Cancer Res 2010; 70:1635-44.). Thus, the lethality of a Cav-1-deficient tumor microenvironment could be explained by an autophagic/catabolic tumor stroma, which would then provide both nutrients and energy to epithelial cancer cells in a paracrine fashion. This is the “autophagic tumor stroma model of cancer”. This represents a unique therapeutic opportunity, as blocking autophagy in the tumor stroma should halt cancer growth, while an induction of autophagy in the epithelial cancer cells should have the same effect, thereby halting tumor growth. This new model of compartmentalized autophagy clarifies and explains the controversial role of autophagy in tumor pathogenesis and facilitate the design of novel anti-cancer therapies.

Based on these and other supporting findings, this is a new model for understanding the Warburg effect in tumor metabolism. In this model, epithelial cancer cells induce aerobic glycolysis in adjacent cancer-associated fibroblasts, directing them to produce energy-rich metabolites (such as lactate and 3-hydroxy-butryate). Then, these metabolites would be transferred to the epithelial cancer cells, where they can then enter the mitochondrial TCA cycle, undergo oxidative phosphorylation, resulting high ATP production. The inventors have termed this new model “The Reverse Warburg Effect” Pavlides S, Whitaker-Menezes D, Castello-Cros R, Flomenberg N, Witkiewicz A K, Frank P G, Casimiro M C, Wang C, Fortina P, Addya S, Pestell R G, Martinez-Outschoorn U E, Sotgia F, Lisanti M P. The reverse Warburg effect: aerobic glycolysis in cancer associated fibroblasts and the tumor stroma. Cell Cycle 2009; 8:3984-4001; Pavlides S, Tsirigos A, Vera I, Flomenberg N, Frank P G, Casimiro M C, Wang C, Pestell R G, Martinez-Outschoorn U E, Howell A, Sotgia F, Lisanti M P. Transcriptional evidence for the “Reverse Warburg Effect” in human breast cancer tumor stroma and metastasis: similarities with oxidative stress, inflammation, Alzheimer's disease, and “Neuron-Glia Metabolic Coupling”. Aging (Albany N.Y.) 2010; 2:185-99.

In direct support of these findings, it was recently shown using a co-culture model, that MCF7 epithelial cancer cells have the ability to down-regulate both Cav-1 expression and mitochondria in adjacent fibroblasts via the induction of autophagy/mitophagy. This then drives aerobic glycolysis in the fibroblast compartment. More specifically, MCF7 cells induce oxidative stress in adjacent fibroblasts. Oxidative stress is then sufficient to drive the induction of autophagy/mitophagy in fibroblasts, leading to Cav-1 lysosomal degradation and aerobic glycolysis. Conversely, during co-culture, it was observed that MCF7 epithelial cancer cells dramatically increase their mitochondrial mass and mitochondrial activity 11. Moreover, it was possible to pheno-copy these effects by simply adding L-lactate (an end product of glycolysis) to the tissue culture media of MCF7 cells. Under these conditions, L-lactate treatment was sufficient to dramatically increase mitochondrial mass in MCF7 cancer cells. Martinez-Outschoorn U E, Balliet R, Rivadeneira D, Chiavarina B, Pavlides S, Wang C, Whitaker-Menezes D, Daumer K M, Lin Z, Witkiewicz A K, Flomenberg N, Howell A, Pestell R G, Knudsen E, Sotgia F, Lisanti M P. Oxidative stress in cancer fibroblasts drives tumor-stroma co-evolution: A new paradigm for understanding tumor metabolism, the field effect and genomic instability in cancer cells. Cell Cycle 2010; 9: In Press; Martinez-Outschoom U E, Pavlides S, Whitaker-Menezes D. Daumer K M, Milliman J N, Chiavarina B, Migneco G, Witkiewicz A K, Martinez-Cantarin M P. Flomenberg N, Howell A, Pestell R G, Lisanti M P, Sotgia F. Tumor cells induce the cancer associated fibroblast phenotype via caveolin-1 degradation: Implications for breast cancer and DCIS therapy with autophagy inhibitors. Cell Cycle 2010; 9: In Press.

Based on the above biomarker and mechanistic experiments, the Cav-1 (−/−) mammary fat pad can be used as a pre-clinical model of a “lethal” tumor microenvironment. Pavlides S, Tsirigos A, Vera I, Flomenberg N, Frank P G, Casimiro M C, Wang C, Fortina P, Addya S, Pestell R G, Rigoutsos I, Martinez-Outschoom U E, Sotgia F, Lisanti M P. Loss of Stromal Caveolin-1 Leads to Oxidative Stress, Mimics Hypoxia, and Drives Inflammation in the Tumor Microenvironment, Conferring the “Reverse Warburg Effect”: A Transcriptional Informatics Analysis with Validation. Cell Cycle 2010; In Press. With this in mind, Cav-1 (−/−) null mammary fat pads were subjected to an unbiased metabolomics analysis. The results obtained provided independent validation for the idea that a loss of stromal Cav-1 induces oxidative stress, which in turn activates autophagy/mitophagy, leading to aerobic glycolysis. Importantly, 3-hydroxy-butyrate (a ketone body) is one of the key metabolites that was most significantly elevated, over 4-fold. 3-Hydroxy-butyrate is another metabolic end-product of glycolysis (which can be derived from pyruvate) that accumulates during starvation, and mitochondrial dysfunction, and is elevated in diabetic patients. Pavlides S, Tsirigos A, Migneco G, Whitaker-Menezes D, Chiavarina B, Flomenberg N, Frank P G, Casimiro M C, Wang C, Pestell R G, Martinez-Outschoorn U E, Howell A, Sotgia F, Lisanti M P. The Autophagic Tumor Stroma Model of Cancer: Role of Oxidative Stress and Ketone Production in Fueling Tumor Cell Metabolism. Cell Cycle 2010: Submitted.

There are several important parallels between 3-hydroxy-butyrate and L-lactate. Both can be considered metabolic end-products of glycolysis, derived from pyruvate. 3-hydroxy-butyrate and L-lactate are both secreted and take up by the same monocarboxylate transporters (MCTs). After uptake by MCTs, they can both re-enter the TCA cycle as acetyl-CoA and undergo oxidative metabolism, resulting the production of high levels of ATP. Thus, based on these findings, both ketones and lactate (produced via aerobic glycolysis in fibroblasts) could fuel tumor growth and metastasis in epithelial cancer cells.

Here, a xenograft model of human breast cancer was used to assess the possible tumor promoting properties of the end-products of aerobic glycolysis, namely ketones and lactate. For this purpose, MDA-MB-231 human breast cancer cells, which show a marker profile most consistent with triple negative and basal-like breast cancers. MDA-MB-231 cells were grown in athymic nude mice as solid tumors via flank injections, or were induced to undergo lung metastasis via tail vein injections. Then, 3-hydroxy-butyrate or L-lactate was systemically administered via intra-peritoneal (i.p.) injections. Our results clearly show that 3-hydroxy-butyrate or L-lactate “fuel” tumor growth and metastasis, without a measurable increase in tumor angiogenesis. Thus, our results provide metabolic/functional evidence to directly support “The Reverse Warburg Effect”. Via an informatics analysis of the existing raw transcriptional profiles of epithelial cancer cells and adjacent stromal cells, the inventors also provide evidence for the upregulation of oxidative phosphorylation, the TCA cycle, and mitochondrial metabolism in human breast cancer cells in vivo. Casey T, Bond J, Tighe S, Hunter T, Lintault L, Patel O, Eneman J, Crocker A, White J, Tessitore J, Stanley M, Harlow S. Weaver D, Muss H, Plaut K. Molecular signatures suggest a major role for stromal cells in development of invasive breast cancer. Breast Cancer Res Treat 2009; 114:47-62.

All references cited herein are incorporated herein by reference in their entireties.

### BRIEF SUMMARY OF THE INVENTION

The invention provides a method for making a prognosis of disease course in a human neoplastic disease patient, the method comprising the steps of (a) obtaining a sample of stromal cells adjacent to a neoplasm, stromal cells within a neoplasm, and/or a total tumor extract; (b) determining the level of protein and/or mRNA expression of a biomarker selected from the group consisting of ACLY, HMGCS1, HMGCS2, HMGCL, HMGCLL1, BDH1, BDH2, BNIP3, BNIP3L, and combinations thereof, in the sample; wherein said prognosis is made when the level of biomarker in the sample is higher than the level of biomarker in a control. The invention further provides the method wherein the human neoplastic disease patient has a neoplasm selected from the group consisting of breast, skin, kidney, lung, pancreas, rectum and colon, prostate, bladder, epithelial, non-epithelial, lymphomas, sarcomas, melanomas, and the like. The invention further provides the method wherein the human neoplastic disease patient has a breast neoplasm subtype selected from the group consisting of ER(+), PR(+), HER2(+), triple-negative (ER(−)/PR(−)/HER2(−)), ER(−), PR(−), all tumor and nodal stages, and all tumor grades. The invention further provides a method wherein the level of biomarker expression is determined by immunohistochemical staining. The invention further provides a method wherein the level of biomarker expression is determined by an assay selected from the group consisting of RT-PCR, QRT-PCR, rolling circle amplification and nucleic acid sequenced-based amplification assays. The invention further provides a method wherein the prognosis of disease course includes a risk for metastasis, recurrence and relapse of neoplastic disease. The invention further provides a method wherein increase of biomarker predicts early disease recurrence, metastasis, survival, and tamoxifen-resistance at diagnosis. The invention further provides a method wherein increase of biomarker in the sample predicts the prognosis of lymph-node positive (LN(+)) patients. The invention further provides a method wherein increase of biomarker in the sample is associated with a poor prognosis. The invention further provides a method wherein the up-regulation or presence of biomarker in the sample is associated with a bad prognosis. The invention further provides a method wherein the neoplasm is a pre-malignant lesions selected from the group consisting of ductal carcinoma in situ (DCIS) of the breast and myelodysplastic syndrome of the bone marrow. The invention further provides a method wherein the prognosis of disease course includes staging malignant disease in a human neoplastic disease patient. The invention further provides a method wherein an increase of biomarker in the sample is a surrogate marker for stromal RB tumor suppressor functional inactivation by RB hyper-phosphorylation.

The invention provides a method for determining the likelihood that a carcinoma is of a grade likely to become an invasive carcinoma comprising: (a) obtaining a sample of stromal cells adjacent to a neoplasm, stromal cells within a neoplasm, and/or a total tumor extract from a neoplastic disease patient; (b) determining the labeling level of protein and/or mRNA expression of a biomarker selected from the group consisting of ACLY, HMGCS1, HMGCS2, HMGCL, HMGCLL1, BDH1, BDH2, BNIP3, BNIP3L, and combinations thereof, in the sample; and (c) correlating the amount of labeling signal in the test sample with a control, wherein the carcinoma is of a grade likely to become invasive when the level of biomarker in the sample is higher than the level of biomarker in a control. The invention further provides a method wherein the carcinoma is a carcinoma of the breast. The invention further provides a method wherein the carcinoma is selected from the group consisting of carcinoma of the breast, skin, kidney, parotid gland, lung, bladder and prostate. The invention further provides a method wherein the detection reagent is labeled antibody capable of binding to human ACLY, HMGCS1, HMGCS2, HMGCL, HMGCLL1, BDH1, BDH2, BNIP3, and/or BNIP3L. The invention further provides a method wherein the detection reagent is labeled nucleic acid capable of binding to human ACLY, HMGCS1, HMGCS2, HMGCL, HMGCLL1, BDH1, BDH2, BNIP3, and/or BNIP3L. The invention further provides a method wherein the amount of labeling signal is measured by a technique selected from the group consisting of emulsion autoradiography, phosphorimaging, light microscopy, confocal microscopy, multi-photon microscopy, and fluorescence microscopy. The invention further provides a method wherein the amount of labeling signal is measured by autoradiography and a higher signal intensity in a test sample compared to a control prepared using the same steps as the test sample is used to diagnose a high grade carcinoma possessing a high probability the carcinoma will progress to an invasive carcinoma. The invention further provides a method wherein the amount of labeling signal is measured by a technique selected from the group consisting of is an assay selected from the group consisting of RT-PCR, QRT-PCR, rolling circle amplification and nucleic acid sequenced-based amplification assays.

The invention provides a kit for making a prognosis of disease course in a human neoplastic disease patient, comprising: (a) at least one label that labels a biomarker selected from the group consisting of ACLY, HMGCS1, HMGCS2, HMGCL, HMGCLL1, BDH1, BDH2, BNIP3, BNIP3L, and combinations thereof; and (b) a usage instruction for performing a screening of a sample of said subject with said label such as that an amount of biomarker present in the sample is determined. The invention further provides a kit wherein the subject is a mammal. The invention further provides a kit wherein the subject is a human. The invention further provides a kit wherein the label comprises an antibody that specifically binds to biomarker. The invention further provides a kit wherein the antibody is a monoclonal antibody. The invention further provides a kit wherein the antibody is a polyclonal antibody. The invention further provides a kit further comprising a multiwell plate.

The invention provides a kit for making a prognosis of disease course in a human neoplastic disease patient, comprising: (a) at least one label that labels the protein expression of a protein selected from the group consisting of ACLY, HMGCS1, HMGCS2, HMGCL, HMGCLL1, BDH1, BDH2, BNIP3, BNIP3L, and combinations thereof; and (b) a usage instruction for performing a screening of a sample of said subject with said label such as that an amount of the protein expression of the protein. The invention further provides a kit wherein the subject is a mammal. The invention further provides a kit wherein the subject is a human. The invention further provides a kit wherein the label comprises an antibody that specifically binds to the protein. The invention further provides a kit wherein the antibody is a monoclonal antibody. The invention further provides a kit wherein the antibody is a polyclonal antibody. The invention further provides a kit further comprising a multiwell plate.

The invention provides a kit for making a prognosis of disease course in a human neoplastic disease patient, comprising: (a) a collection of isolated polynucleotides which bind selectively to the RNA products of biomarkers, wherein the biomarkers are selected from the group of genes consisting of ACLY, HMGCS1, HMGCS2, HMGCL, HMGCLL1, BDH1, BDH2, BNIP3, BNIP3L, and combinations thereof; (b) a usage instruction for performing a screening of a sample of said patient with said label such as that an amount of the mRNA expression of biomarker present in the sample is determined. The invention further provides a kit wherein the screening is a nucleic acid amplification assay selected from the group consisting of RT-PCR, QRT-PCR, rolling circle amplification and nucleic acid sequenced-based amplification assays. The invention further provides a kit wherein the subject is a mammal. The invention further provides a kit wherein the subject is a human. The invention further provides a kit wherein the label comprises a nucleic acid that specifically binds to biomarker. The invention further provides a kit further comprising a multiwell plate.

The invention provides a method of predicting response to anti-neoplasm therapy or predicting disease progression neoplastic disease, the method comprising: (a) obtaining a sample of stromal cells adjacent to a neoplasm, stromal cells within a neoplasm, and/or a total tumor extract from a neoplastic disease patient; (b) determining the labeling level of protein and/or mRNA expression of a biomarker selected from the group consisting of ACLY, HMGCS1, HMGCS2, HMGCL, HMGCLL1, BDH1, BDH2, BNIP3, BNIP3L, and combinations thereof, in the sample and comparing the labeling level of biomarker in the sample with the labeling level of biomarker in a control; (c) analyzing the obtained neoplasm test sample for presence or amount of one or more molecular markers of hormone receptor status, one or more growth factor receptor markers, and one or more tumor suppression/apoptosis molecular markers; (d) analyzing one or more additional molecular markers both proteomic and non-proteomic that are indicative of cancer disease processes selected from the group consisting of angiogenesis, apoptosis, catenin/cadherin proliferation/differentiation, cell cycle processes, cell surface processes, cell-cell interaction, cell migration, centrosomal processes, cellular adhesion, cellular proliferation, cellular metastasis, invasion, cytoskeletal processes, ERBB2 interactions, estrogen co-receptors, growth factors and receptors, membrane/integrin/signal transduction, metastasis, oncogenes, proliferation, proliferation oncogenes, signal transduction, surface antigens and transcription factor molecular markers; and then correlating (b) the presence or amount of biomarker, with (d) clinicopathological data from said tissue sample other than the molecular markers of cancer disease processes, in order to ascertain a probability of response to therapy or future risk of disease progression in cancer for the subject. The invention further provides a method wherein the human neoplastic disease patient has a breast neoplasm subtype selected from the group consisting of ER(+), PR(+), HER2(+), triple-negative (ER(−)/PR(−)/HER2(−)), ER(−), PR(−), all tumor and nodal stages, and all tumor grades. The invention further provides a method wherein the human neoplastic disease patient has a neoplasm selected from the group consisting of breast, skin, kidney, lung, pancreas, rectum and colon, prostate, bladder, epithelial, non-epithelial, lymphomas, sarcomas, melanomas, and the like. The invention further provides a method wherein the neoplasm is a pre-malignant lesions selected from the group consisting of ductal carcinoma in situ (DCIS) of the breast and myelodysplastic syndrome of the bone marrow. The invention further provides a method wherein the correlating to ascertain a probability of response to a specific anti-neoplasm therapy drawn from the group consisting of tamoxifen, anastrozole, letrozole or exemestane. The invention further provides a method wherein the one or more additional markers includes, in addition to markers ER, PR, and/or HER-2. The invention further provides a method wherein the one or more additional markers includes, in addition to markers ER, PR, and/or HER-2. The invention further provides a method wherein the neoplasm is breast cancer. The invention further provides a method wherein the analyzing is of both proteomic and clinicopathological markers; and wherein the correlating is further so as to a clinical detection of disease, disease diagnosis, disease prognosis, or treatment outcome or a combination of any two, three or four of these actions. The invention further provides a method wherein the obtaining of the test sample from the subject is of a test sample selected from the group consisting of fixed, paraffin-embedded tissue, breast cancer tissue biopsy, tissue microarray, fresh neoplasm tissue, fine needle aspirates, peritoneal fluid, ductal lavage and pleural fluid or a derivative thereof. The invention further provides a method wherein the molecular markers of estrogen receptor status are ER and PGR, the molecular markers of growth factor receptors are ERBB2, and the tumor suppression molecular markers are TP-53 and BCL-2; wherein the additional one or more molecular marker(s) is selected from the group consisting of essentially: ER, PR, HER-2, MKI67, KRT5/6, MSN, C-MYC, CAV1, CTNNB1, CDH1, MME, AURKA, P-27, GATA3, HER4, VEGF, CTNNA1, and/or CCNE; wherein the clinicopathological data is one or more datum values selected from the group consisting essentially of: tumor size, nodal status, and grade; wherein the correlating is by usage of a trained kernel partial least squares algorithm; and the prediction is of outcome of anti-neoplasm therapy for breast cancer.

The invention provides a kit comprising: a panel of antibodies comprising: at least one antibody or binding fragment thereof specific for a biomarker selected from the group consisting of ACLY, HMGCS1, HMGCS2, HMGCL, HMGCLL1, BDH1, BDH2, BNIP3, BNIP3L, and combinations thereof, whose binding with stromal cells adjacent to a neoplasm and/or total tumor extract has been correlated with breast cancer treatment outcome or patient prognosis; at least one additional antibody or binding fragment thereof specific for a protein whose expression is correlated with breast cancer treatment outcome or patient prognosis, reagents to perform a binding assay; a computer algorithm, residing on a computer, operating, in consideration of all antibodies of the panel historically analyzed to bind to samples, to interpolate, from the aggregation of all specific antibodies of the panel found bound to the stromal cells adjacent to a neoplasm sample, a prediction of treatment outcome for a specific treatment for breast cancer or a future risk of breast cancer progression for the subject. The invention further provides a kit wherein the anti-biomarker antibody comprises: a poly- or monoclonal antibody specific for biomarker protein or protein fragment thereof correlated with breast cancer treatment outcome or patient prognosis. The invention further provides a kit wherein the panel of antibodies further comprises: a number of immunohistochemistry assays equal to the number of antibodies within the panel of antibodies. The invention further provides a kit wherein the antibodies of the panel of antibodies further comprise: antibodies specific to ER, PR, and/or HER-2. The invention further provides a kit wherein the treatment outcome predicted comprises the response to anti-neoplastic therapy or chemotherapy. The invention further provides a kit further comprising a multiwell plate.

The invention provides a method for making a prognosis of disease course in a human patient by detecting differential expression of at least one marker in ductal carcinoma in situ (DCIS) pre-invasive cancerous breast tissue, said method comprising the steps of: (a) obtaining a sample of DCIS breast tissue and stromal cells adjacent to a neoplasm, stromal cells within a neoplasm, and/or a total tumor extract from a human neoplastic disease patient; (b) determining the level of protein and/or mRNA expression of a biomarker selected from the group consisting of ACLY, HMGCS1, HMGCS2, HMGCL, HMGCLL1, BDH1, BDH2, BNIP3, BNIP3L, and combinations thereof, in the stromal cells of the sample as the at least one marker and comparing the level of biomarker in the stromal cells of the sample with the level of biomarker in a control; wherein said prognosis of further progression is made when the level of biomarker in the stromal cells of the sample is higher than the level of biomarker in the control. The invention further provides a method wherein the size of said abnormal tissue sample substantially conforms to an isolatable tissue structure wherein only cells exhibiting abnormal cytological or histological characteristics are collected. The invention further provides a method further comprising confirming said differential expression of said marker in said normal tissue sample and in said abnormal tissue sample by using an immunological technique. The invention further provides a method wherein said immunological technique is selected from the group consisting of radioimmunoassay (RIA), EIA, ELISA, and immunofluorescence assays. The invention further provides a method wherein said immunological technique is selected from the group consisting of is determined by an assay selected from the group consisting of RT-PCR. QRT-PCR, rolling circle amplification and nucleic acid sequenced-based amplification assays. The invention further provides a method wherein said abnormal breast tissue cells are non-comedo ductal carcinoma in situ cells. The invention further provides a method wherein the amount of labeling signal is measured by a technique selected from the group consisting of is an assay selected from the group consisting of RT-PCR. QRT-PCR, rolling circle amplification and nucleic acid sequenced-based amplification assays.

The invention provides a method for making a prognosis of disease course in a human neoplastic disease patient, the method comprising the steps of: (a) obtaining a sample of stromal cells adjacent to a neoplasm, stromal cells within a neoplasm, and/or a total tumor extract; (b) determining the level of one or more RNA transcripts expressed in the sample obtained from said patient, wherein said one or more RNA transcripts corresponds to a biomarker selected from the group consisting of miR-31, miR-34c, and combinations thereof; and (c) comparing the level of each of said one or more RNA transcripts in said sample according to step (a) with the level of each of said one or more RNA transcripts in a control sample; (d) comparing the level of each of said one or more RNA transcripts in said sample according to step (a) with the level of each of said one or more RNA transcripts in a control sample; wherein said prognosis is made when the level of one or more RNA transcripts in the stromal cells of the sample is higher than the level of one or more RNA transcripts in a control.

The invention provides a method for making a prognosis of disease course in a human neoplastic disease patient, the method comprising the steps of: (a) obtaining a sample of stromal cells adjacent to a neoplasm, stromal cells within a neoplasm, and/or a total tumor extract; (b) determining the level of a biomarker selected from the group consisting of ADMA, 3-hydroxybutyrate, and combinations thereof, in the sample; wherein said prognosis is made when the level of biomarker in the sample is higher than the level of biomarker in a control. The invention further provides a method wherein the human neoplastic disease patient has a neoplasm selected from the group consisting of breast, skin, kidney, lung, pancreas, rectum and colon, prostate, bladder, epithelial, non-epithelial, lymphomas, sarcomas, melanomas, and the like. The invention further provides a method wherein the human neoplastic disease patient has a breast neoplasm subtype selected from the group consisting of ER(+), PR(+), HER2(+), triple-negative (ER(−)/PR(−)/HER2(−)), ER(−), PR(−), all tumor and nodal stages, and all tumor grades.

The invention provides a method for making a prognosis of disease course in a human neoplastic disease patient, the method comprising the steps of: (a) obtaining a sample of stromal cells adjacent to a neoplasm, stromal cells within a neoplasm, and/or a total tumor extract; (b) determining the level of protein and/or mRNA expression of a biomarker selected from the group consisting of ACAT1, ACAT2, OXCT1, OXCT2, and combinations thereof, in the sample; wherein said prognosis is made when the level of biomarker in the sample is higher than the level of biomarker in a control. The invention further provides a method wherein the human neoplastic disease patient has a neoplasm selected from the group consisting of breast, skin, kidney, lung, pancreas, rectum and colon, prostate, bladder, epithelial, non-epithelial, lymphomas, sarcomas, melanomas, and the like. The invention further provides a method wherein the human neoplastic disease patient has a breast neoplasm subtype selected from the group consisting of ER(+), PR(+), HER2(+), triple-negative (ER(−)/PR(−)/HER2(−)), ER(−), PR(−), all tumor and nodal stages, and all tumor grades. The invention further provides a method wherein the level of biomarker is determined by immunohistochemical staining. The invention further provides a method wherein the level of biomarker is determined by an assay selected from the group consisting of RT-PCR, QRT-PCR, rolling circle amplification and nucleic acid sequenced-based amplification assays. The invention further provides a method wherein the prognosis of disease course includes a risk for metastasis, recurrence and relapse of neoplastic disease. The invention further provides a method wherein increase of biomarker predicts early disease recurrence, metastasis, survival, and tamoxifen-resistance at diagnosis. The invention further provides a method wherein increase of biomarker predicts the prognosis of lymph-node positive (LN(+)) patients. The invention further provides a method wherein increase of biomarker is associated with a poor prognosis. The invention further provides a method wherein the up-regulation or presence of biomarker is associated with a bad prognosis. The invention further provides a method wherein the neoplasm is a pre-malignant lesions selected from the group consisting of ductal carcinoma in situ (DCIS) of the breast and myelodysplastic syndrome of the bone marrow. The invention further provides a method wherein the prognosis of disease course includes staging malignant disease in a human neoplastic disease patient. The invention further provides a method wherein increase of biomarker is a surrogate marker for stromal RB tumor suppressor functional inactivation by RB hyper-phosphorylation.

The invention provides a method for making a prognosis of disease course in a human neoplastic disease patient, the method comprising the steps of: (a) obtaining a sample of a body fluid from the patient; (b) determining the level of a biomarker selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof, in the sample; wherein said prognosis is made when the level of biomarker in sample is higher than the level of biomarker in a control. The invention further provides a method wherein the human neoplastic disease patient has a neoplasm selected from the group consisting of breast, skin, kidney, lung, pancreas, rectum and colon, prostate, bladder, epithelial, non-epithelial, lymphomas, sarcomas, melanomas, and the like. The invention further provides a method wherein the human neoplastic disease patient has a breast neoplasm subtype selected from the group consisting of ER(+), PR(+), HER2(+), triple-negative (ER(−)/PR(−)/HER2(−)), ER(−), PR(−), all tumor and nodal stages, and all tumor grades. The invention further provides a method wherein the level of biomarker is determined by immunohistochemical staining. The invention further provides a method wherein the level of biomarker is determined by an assay selected from the group consisting of RT-PCR, QRT-PCR, rolling circle amplification and nucleic acid sequenced-based amplification assays. The invention further provides a method wherein the level of biomarker is determined by enzymatic assay. The invention further provides a method wherein the prognosis of disease course includes a risk for metastasis, recurrence and relapse of neoplastic disease. The invention further provides a method wherein increase of biomarker predicts early disease recurrence, metastasis, survival, and tamoxifen-resistance at diagnosis. The invention further provides a method wherein increase of biomarker in the sample predicts the prognosis of lymph-node positive (LN(+)) patients. The invention further provides a method wherein increase of biomarker in the sample is associated with a poor prognosis. The invention further provides a method wherein the up-regulation or presence of biomarker in the sample is associated with a bad prognosis. The invention further provides a method wherein the neoplasm is a pre-malignant lesions selected from the group consisting of ductal carcinoma in situ (DCIS) of the breast and myelodysplastic syndrome of the bone marrow. The invention further provides a method wherein the prognosis of disease course includes staging malignant disease in a human neoplastic disease patient. The invention further provides a method wherein increase of biomarker in the sample is a surrogate marker for stromal RB tumor suppressor functional inactivation by RB hyper-phosphorylation. The invention further provides a method wherein the body fluid is selected from the group consisting of plasma, serum, blood, lymphatic fluid, cerebrospinal fluid, synovial fluid, urine, saliva, mucous, phlegm, sputum, and combinations thereof.

The invention provides a method for treating neoplastic disease in a patient, comprising the steps of: (a) obtaining a sample of stromal cells adjacent to a neoplasm, stromal cells within a neoplasm, and/or a total tumor extract from the neoplastic disease patient; (b) determining the level of a biomarker selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof, in the sample and comparing the level of biomarker in the sample with the level of biomarker in a control; (c) predicting if the neoplasm will respond effectively to treatment with an anti-angiogenic agent, wherein said prediction is made when the level of biomarker in the sample is higher than the level of biomarker in the control; and administering to said patient a therapeutically effective amount of an anti-angiogenic agent. The invention further provides a method wherein the anti-angiogenic agent comprises an agent selected from the group consisting of angiostatin, bevacizumab, arresten, canstatin, combretastatin, endostatin, NM-3, thrombospondin, tumstatin, 2-methoxyestradiol, Vitaxin, Getfitinib, ZD6474, erlotinib, CI1033, PKI1666, cetuximab, PTK787, SU6668, SU11248, trastuzumab, Marimastat, COL-3, Neovastat, 2-ME, SU6668, anti-VEGF antibody, Medi-522 (Vitaxin II), tumstatin, arrestin, recombinant EPO, troponin I, EMD121974, IFN, celecoxib, PD0332991, and thalidomide. The invention further provides a method wherein one or more additional anti-neoplastic agents are co-administered simultaneously or sequentially with the anti-angiogenic agent. The invention further provides a method wherein the at least one or more additional anti-neoplastic agent comprises a proteasome inhibitor. The invention further provides a method wherein the proteasome inhibitor is bortezomib. The invention further provides a method wherein the human neoplastic disease patient has a breast neoplasm subtype selected from the group consisting of ER(+), PR(+), HER2(+), triple-negative (ER(−)/PR(−)/HER2(−)), ER(−), PR(−), all neoplasm and nodal stages, and all neoplasm grades. The invention further provides a method wherein the human neoplastic disease patient has a neoplasm selected from the group consisting of breast, skin, kidney, lung, pancreas, rectum and colon, prostate, bladder, epithelial, non-epithelial, lymphomas, sarcomas, melanomas, and the like. The invention further provides a method wherein the neoplasm is a pre-malignant lesion selected from the group consisting of ductal carcinoma in situ (DCIS) of the breast and myelodysplastic syndrome of the bone marrow.

The invention provides a diagnostic kit for assaying the individual sensitivity of target cells towards angiogenesis inhibitors, comprising: (a) at least one molecule that specifically binds to a biomarker selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxy butyrate, and combinations thereof; and (b) a pharmaceutically acceptable carrier.

The invention provides a method of predicting whether a neoplastic disease patient is afflicted with a neoplasm that will respond effectively to treatment with an anti-angiogenic agent, comprising: (a) obtaining a sample of stromal cells adjacent to a neoplasm, stromal cells within a neoplasm, and/or a total tumor extract from the neoplastic disease patient; (b) determining the level of a biomarker selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof, in the sample and comparing the level of biomarker in the sample with the level of biomarker in a control; (c) predicting if the neoplasm will respond effectively to treatment with an anti-angiogenic agent, wherein higher expression levels of biomarker in the sample relative to biomarker levels in the control correlate with a neoplasm that will respond effectively to treatment with an anti-angiogenic agent. The invention further provides a method wherein the anti-angiogenic agent comprises an agent selected from the group consisting of angiostatin, bevacizumab, arresten, canstatin, combretastatin, endostatin, NM-3, thrombospondin, tumstatin, 2-methoxyestradiol, Vitaxin, Getfitinib, ZD6474, erlotinib, CI1033, PKI1666, cetuximab, PTK787, SU6668, SU11248, trastuzumab, Marimastat, COL-3, Neovastat, 2-ME, SU6668, anti-VEGF antibody, Medi-522 (Vitaxin II), tumstatin, arrestin, recombinant EPO, troponin I, EMD121974, IFN, celecoxib, PD0332991, and thalidomide.

The invention provides a method of predicting the sensitivity of neoplasm cell growth to inhibition by an anti-neoplastic agent, comprising: (a) obtaining a sample of stromal cells adjacent to a neoplasm, stromal cells within a neoplasm, and/or a total tumor extract from a neoplastic disease patient; (b) determining a level of a biomarker selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof, in the sample and comparing the level of biomarker in the sample with the level of biomarker in a control; and (c) predicting the sensitivity of neoplasm cell growth to inhibition by an anti-neoplastic agent, wherein higher levels of the biomarker compared the level of biomarker in a control correlates with high sensitivity to inhibition by anti-neoplastic agent. The invention further provides a method wherein the anti-angiogenic agent comprises an agent selected from the group consisting of angiostatin, bevacizumab, arresten, canstatin, combretastatin, endostatin, NM-3, thrombospondin, tumstatin, 2-methoxyestradiol, Vitaxin, Getfitinib, ZD6474, erlotinib, CI183J, 033, PKI1666, cetuximab, PTK787, SU6668, SU11248, trastuzumab, Marimastat, COL-3, Neovastat, 2-ME, SU6668, anti-VEGF antibody, Medi-522 (Vitaxin II), tumstatin, arrestin, recombinant EPO, troponin I, EMD121974, IFN, celecoxib, PD0332991, and thalidomide. The invention further provides a method wherein the angiogenesis inhibitor is selected from the group consisting of angiostatin, bevacizumab, arresten, canstatin, combretastatin, endostatin, NM-3, thrombospondin, tumstatin, 2-methoxyestradiol, Vitaxin, Getfitinib, ZD6474, erlotinib, CI1033, PKI1666, cetuximab, PTK787, SU6668, SU11248, trastuzumab, Marimastat, COL-3, Neovastat, 2-ME, SU6668, anti-VEGF antibody, Medi-522 (Vitaxin II), tumstatin, arrestin, recombinant EPO, troponin I, EMD121974, IFN, celecoxib, PD0332991, and thalidomide. The invention further provides a diagnostic kit wherein the target cell is a cancer cell.

The invention provides a kit for determining target neoplastic cells susceptible to anti-angiogenesis inhibitor treatment, comprising: (a) at least one antibody which specifically binds a biomarker selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof; and (b) a pharmaceutically acceptable carrier. The invention further provides a diagnostic kit wherein the antibody is a polyclonal antibody. The invention further provides a diagnostic kit wherein the antibody is a monoclonal antibody.

The invention provides a kit for determining target neoplastic cells susceptible to anti-angiogenesis inhibitor treatment, comprising: (a) a collection of isolated polynucleotides which bind selectively to the RNA products of biomarkers, wherein the biomarkers are selected from the group of genes consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof; (b) a usage instruction for performing a screening of a sample of said patient with said label such as that an amount of the mRNA expression of a biomarker present in the sample is determined. The invention further provides a kit wherein the screening is a nucleic acid amplification assay selected from the group consisting of RT-PCR, QRT-PCR, rolling circle amplification and nucleic acid sequenced-based amplification assays. The invention further provides a kit wherein the subject is a mammal. The invention further provides a kit wherein the subject is a human. The invention further provides a kit wherein the label comprises an nucleic acid that specifically binds to a biomarker selected from the group consisting of ACLY, HMGCS1, HMGCS2, HMGCL, HMGCLL1, BDH11, BDH2, BNIP3, BNIP3L, miR-31, miR-34c, ACAT1, ACAT2, OXCT1, OXCT2, ADMA, and 3-hydroxybutyrate.

The invention provides a method for treating neoplastic disease in a patient, comprising the steps of: (a) obtaining a sample of stromal cells adjacent to a neoplasm, stromal cells within a neoplasm, and/or a total tumor extract a neoplasm from the patient and/or total tumor extract; (b) determining the level of a biomarker selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof, in the sample and comparing the level of biomarker in the sample with the level of biomarker in a control; (c) predicting if the neoplasm will respond effectively to treatment with a lactate transporter inhibitor, wherein higher expression levels of the biomarker compared the level of biomarker in a control correlates with high sensitivity to treatment with a lactate transporter inhibitor; and (d) administering to said patient a therapeutically effective amount of a lactate transporter inhibitor. The invention further provides a method wherein the lactate transporter inhibitor comprises an agent which inhibits an enzyme selected from the group consisting of triose-phosphate isomerase, fructose 1,6 bisphosphate aldolase, glycero-3-phosphate dehydrogenase, phosphoglycerate kinase, phosphoglycerate mutase, enolase, pyruvate kinase, lactate dehydrogenase. The invention further provides a method wherein one or more additional anti-neoplastic agents are co-administered simultaneously or sequentially with the lactate transporter inhibitor. The invention further provides a method wherein the human neoplastic disease patient has a breast neoplasm subtype selected from the group consisting of ER(+), PR(+), HER2(+), triple-negative (ER(−)/PR(−)/HER2(−)), ER(−), PR(−), all neoplasm and nodal stages, and all neoplasm grades. The invention further provides a method wherein the human neoplastic disease patient has a neoplasm selected from the group consisting of breast, skin, kidney, lung, pancreas, rectum and colon, prostate, bladder, epithelial, non-epithelial, lymphomas, sarcomas, melanomas, and the like. The invention further provides a method wherein the neoplasm is a pre-malignant lesion selected from the group consisting of ductal carcinoma in situ (DCIS) of the breast and myelodysplastic syndrome of the bone marrow.

The invention provides a method for treating neoplastic disease in a patient, comprising the steps of: (a) obtaining a sample of stromal cells adjacent to a neoplasm, stromal cells within a neoplasm, and/or a total tumor extract a neoplasm from the patient and/or total tumor extract; (b) determining the level of a biomarker selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof, in the sample and comparing the level of biomarker in the sample with the level of biomarker in a control; (c) predicting if the neoplasm will respond effectively to treatment with a monocarboxylate transporter inhibitor, wherein higher expression levels of the biomarker compared the level of biomarker in a control correlates with high sensitivity to treatment with a monocarboxylate transporter inhibitor; and (d) administering to said patient a therapeutically effective amount of a monocarboxylate transporter inhibitor. The invention further provides a method wherein the monocarboxylate transporter inhibitor comprises an agent which inhibits an enzyme selected from the group consisting of triose-phosphate isomerase, fructose 1,6 bisphosphate aldolase, glycero-3-phosphate dehydrogenase, phosphoglycerate kinase, phosphoglycerate mutase, enolase, pyruvate kinase, lactate dehydrogenase. The invention further provides a method wherein one or more additional anti-neoplastic agents are co-administered simultaneously or sequentially with the lactate transporter inhibitor. The invention further provides a method wherein the human neoplastic disease patient has a breast neoplasm subtype selected from the group consisting of ER(+), PR(+), HER2(+), triple-negative (ER(−)/PR(−)/HER2(−)), ER(−), PR(−), all neoplasm and nodal stages, and all neoplasm grades. The invention further provides a method wherein the human neoplastic disease patient has a neoplasm selected from the group consisting of breast, skin, kidney, lung, pancreas, rectum and colon, prostate, bladder, epithelial, non-epithelial, lymphomas, sarcomas, melanomas, and the like. The invention further provides a method wherein the neoplasm is a pre-malignant lesion selected from the group consisting of ductal carcinoma in situ (DCIS) of the breast and myelodysplastic syndrome of the bone marrow. The invention further provides a method wherein the monocarboxylate transporter inhibitor is AR-C117977. The invention provides a method of predicting the sensitivity of neoplasm cell growth to inhibition by a lactate transporter inhibitor, comprising: (a) obtaining a sample of stromal cells adjacent to a neoplasm, stromal cells within a neoplasm, and/or a total tumor extract from a neoplastic disease patient; (b) determining the level of a biomarker selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof, in the sample and comparing the level of biomarker in the sample with the level of biomarker in a control; and (c) predicting the sensitivity of neoplasm cell growth to inhibition by a lactate transporter inhibitor, wherein higher expression levels of the biomarker compared the level of biomarker in a control correlates with high sensitivity to inhibition by a lactate transporter inhibitor. The invention further provides a method wherein the lactate transporter inhibitor comprises an agent which inhibits an enzyme selected from the group consisting of triose-phosphate isomerase, fructose 1,6 bisphosphate aldolase, glycero-3-phosphate dehydrogenase, phosphoglycerate kinase, phosphoglycerate mutase, enolase, pyruvate kinase, lactate dehydrogenase.

The invention provides a method of screening for antineoplastic activity of a potential therapeutic agent comprising: (a) providing a cell expressing of a biomolecule selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, combinations thereof, and fragments thereof; (b) providing a potential therapeutic agent; (c) contacting the cell with the potential therapeutic agent; and (d) monitoring an effect of the potential therapeutic agent on bioactivity and/or expression of the biomolecule in the cell. The invention further provides a method of screening for antineoplastic activity further comprising: (e) comparing the level of bioactivity in the absence of said potential therapeutic agent to the level of expression in the presence of the potential therapeutic agent, wherein a potential therapeutic agent is identified when the bioactivity and/or expression of the biomolecule in the absence of said potential therapeutic agent is different than the level of bioactivity and/or expression in the presence of the candidate bioactive agent. The invention further provides a method of screening for the potential therapeutic agent wherein the potential therapeutic agent affects the expression of the biomolecule selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, combinations thereof, and fragments thereof. The invention further provides a method of screening for the potential therapeutic agent wherein the potential therapeutic agent affects the bioactivity of the biomolecule selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, combinations thereof, and fragments thereof.

The invention provides a method for screening for a potential therapeutic agent capable of modulating the bioactivity and/or expression of a biomolecule selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof, said method comprising: a) combining said biomolecule and a candidate bioactive agent; b) determining the effect of the candidate bioactive agent on the bioactivity and/or expression of said biomolecule; c) comparing the level of bioactivity and/or expression of biomolecule in the absence of said potential therapeutic agent to the level of bioactivity and/or expression in the presence of the candidate bioactive agent, wherein a potential therapeutic agent is identified when the bioactivity and/or expression of the biomolecule in the absence of said potential therapeutic agent is different than the level of bioactivity and/or expression in the presence of the candidate bioactive agent. The invention further provides a method of screening for the bioactive agent wherein the potential therapeutic agent affects the expression of the biomolecule selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof. The invention further provides a method of screening for the bioactive agent wherein the potential therapeutic agent affects the bioactivity of the biomolecule selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof.

The invention provides a method for treating neoplastic disease in a patient, comprising the steps of: (a) obtaining a sample of stromal cells adjacent to a neoplasm, stromal cells within a neoplasm, and/or a total tumor extract from a neoplastic disease patient; (b) determining the level of a biomarker selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof, in the sample and comparing the level of biomarker in the sample with the level of biomarker in a control; (c) predicting if the neoplasm will respond effectively to treatment with a therapeutic agent, wherein higher expression levels of the biomarker compared the level of biomarker in a control correlates with high sensitivity to inhibition by a therapeutic agent; and administering to said patient a therapeutically effective amount of a therapeutic agent. The invention further provides a method wherein the therapeutic agent comprises an agent selected from the group consisting of 17-AAG, AR-C117977, Abraxane, albumin-bound Paclitaxel, Albumin Nanoparticle Paclitaxel, Apatinib, Ascomycin, Axitinib, Bexarotene, Bortezomib, Bosutinib, Bryostatin 1, Bryostatin 2, Canertinib, Carboplatin, Cediranib, Cisplatin, Cyclopamine, Dasatinib, 17-DMAG, Docetaxel, Doramapimod, Dovitinib, Erlotinib, Everolimus, Gefitinib, Geldanamycin, Gemeitabine, Imatinib, Imiquimod, Ingenol 3-Angelate, Ingenol 3-Angelate 20-Acetate, Irinotecan, Lapatinib, Lestaurtinib, Nedaplatin, Masitinib, Mubritinib, Nilotinib, NVP-BEZ235, OSU-03012, Oxaliplatin, Paclitaxel, Pazopanib, Picoplatin, Pimecrolimus, PKC412, Rapamycin, Satraplatin, Sorafenib, Sunitinib, Tandutinib, Tivozanib, Thalidomide, Temsirolimus, Tozasertib, Vandetanib, Vargatef, Vatalanib, Zotarolimus, ZSTK474, Bevacizumab (Avasti), Cetuximab, Herceptin, Rituximab, Trastuzumab, Apatinib, Axitinib, Bisindolylmaleimide I, Bisindolylmaleimide I, Bosutinib, Canertinib, Cediranib, Chelerythrine, CP690550, Dasatinib, Dovitinib, Erlotinib, Fasudil, Gefitinib, Genistein, Go 6976, H-89, HA-1077. Imatinib, K252a, K252c, Lapatinib, Di-p-Toluenesulfonate, Lestaurtinib, LY 294002, Masitinib, Mubritinib, Nilotinib, OSU-03012, Pazopanib, PD 98059, PKC412, Roscovitine, SB 202190, SB 203580, Sorafenib, SP600125, Staurosporine, Sunitinib, Tandutinib, Tivozanib, Tozasertib, Tyrphostin AG 490, Tyrphostin AG 1478, U0126, Vandetanib, Vargatef, Vatalanib, Wortmannin, ZSTK474, Cyclopamine, Carboplatin, Cisplatin, Eptaplatin, Nedaplatin, Oxaliplatin, Picoplatin, Satraplatin, Bortezomib (Velcade), Metformin, Halofuginone, Metformin, N-acetyl-cysteine (NAC), RTA 402 (Bardoxolone methyl), Auranofin, BMS-345541, IMD-0354, PS-1145, TPCA-1, Wedelolactone, Echinomycin, 2-deoxy-D-glucose (2-DG), 2-bromo-D-glucose, 2-fluoro-D-glucose, and 2-iodo-D-glucose, dichloro-acetate (DCA), 3-chloro-pyruvate, 3-Bromo-pyruvate (3-BrPA), 3-Bromo-2-oxopropionate, Oxamate, LY 294002, NVP-BEZ235, Rapamycin, Wortmannin, Quercetin, Resveratrol, N-acetyl-cysteine (NAC), N-acetyl-cysteine amide (NACA), Ascomycin, CP690550, Cyclosporin A, Everolimus, Fingolimod, FK-506, Mycophenolic Acid, Pimecrolimus, Rapamycin, Temsirolimus, Zotarolimus, Roscovitine, PD 0332991 (CDK4/6 inhibitor), Chloroquine, BSI-201, Olaparib, DR 2313, and NU 1025.

### BRIEF DESCRIPTION OF SEVERAL VIEWS OF THE DRAWINGS

The invention will be described in conjunction with the following drawings wherein:

FIG. 1 shows Evidence for Oxidative Stress and Mitochondrial Dysfunction in Cav-1 (−/−) Null Mouse Tissues: ADMA and Ketones. Note that both 3-hydroxybuty, rate (BHBA) and asymmetric dimethyl arginine (ADMA) are increased ˜3-4 fold in Cav-1 (−/−) mammary fat pads. Similar results were obtained with lung tissue harvested from Cav-1 (−/−) mice. Importantly, ADMA is a marker of endothelial dysfunction and oxidative stress; it can also drive oxidative stress, as it functions as an uncoupler of NOS family member, inhibiting the production NO and producing superoxide instead. In addition, BHBA is a ketone body known to be a marker of mitochondrial dysfunction. Oxidative stress induces mitochrondrial dysfunction, and visa versa, driving autophagy and mitophagy.

FIG. 2 shows Upregulation of Anti-Oxidants in Cav-1 (−/−) Mammary Fat Pads. One compensatory response to oxidative stress is the over-production of anti-oxidants. Note that in Cav-1 (−/−) mammary fat pads an 11-fold increase in Vitamin C (ascorbic acid) and a near 3-fold increase in Vitamin E (alpha-tocopherol) were observed.

FIG. 3 shows Venn Diagrams for the Transcriptional Overlap Between Autophagy and Tumor Stroma from Breast Cancer Patients. Upper panel, Overlap with tumor stroma. Note the overlap of 93 genes with a p-value of 2.65×10-6. Middle panel, Overlap with “recurrence-prone” stroma. Note the overlap of 47 genes with a p-value of 2.22×10-3. Lower panel, Overlap with “metastasis-prone” stroma. Note the overlap of 17 genes with a p-value of 5.32×10-2.

FIG. 4 shows Venn Diagrams for the Transcriptional Overlap Between Lysosomes and Telomere-related Genes, with Tumor Stroma from Breast Cancer Patients. Upper panel, A, Overlap with tumor stroma. Note the overlap of 175 genes with a p-value of 1.23×10-15. Middle panel, Overlap with “recurrence-prone” stroma. Note the overlap of 74 genes with a p-value of 2.10×10-3. Lower panel, B, Overlap with “metastasis-prone” stroma. Note the overlap of 38 genes with a p-value of 9.67×10-5.

FIG. 5 shows Venn Diagrams for the Transcriptional Overlap Between Peroxisomes and Tumor Stroma from Breast Cancer Patients. Upper panel, Overlap with tumor stroma. Note the overlap of 204 genes with a p-value of 4.25×10-12. Lower panel, Overlap with “recurrence-prone” stroma. Note the overlap of 101 genes with a p-value of 2.76×10-5.

FIG. 6 shows Over-Expression of Autophagy and Mitophagy Markers in Cav-1 (−/−) Null Mammary Fat Pads: Cathepsin B and BNIP3. To validate the idea that a loss of Cav-1 drives the onset of autophagy, the expression of established autophagy markers was assessed, namely cathepsin B and BNIP3, in Cav-1 (−/−) mammary fat pads. Cathepsin B is a well-known lysosomal protease. BNIP3 is a marker of autophagy that mediates the autophagic destruction of mitochondria. Note that both cathepsin B (the pro-enzyme and activated form) and BNIP3 are significantly over-expressed in Cav-1 (−/−) null mammary fat pads (KO), relative wild-type controls (WT). Immuno-blotting with Cav-1 and beta-actin are shown for comparison purposes.

FIG. 7 shows A Lethal Tumor Micro-Environment: Oxidative Stress Drives Stromal Autophagy/Mitophagy, Providing Stromal-Derived Nutrients for Epithelial Cancer Cells. Here, using metabolic, transcriptional mRNA, and miR profiling, it was found that loss of stromal Cav-1 induces oxidative stress, mitochondrial dysfunction, and autophagy/mitophagy in the tumor micro-environment. This model would then provide recycled chemical building blocks (nutrients, amino acids, energy-rich metabolites, nucleotides) derived from stromal cells (fibroblasts) that then could be harnessed by epithelial cancer cells to promote tumor growth. Mitochondrial dysfunction and mitophagy would result in aerobic glycolysis in stromal cells, explaining our previous observations on the “Reverse Warburg Effect”. Many of the key components identified here through metabolic and micro-RNA profiling are shown in BOLD: miR-31, miR-34c, ADMA, essential amino acids (AA's), nucleotides, pyruvate, BHB, and TCA cycle intermediates.

FIG. 8 shows Ketones Can Fuel Tumor Growth. Ketones produced in the tumor micro-environment (in cancer associated fibroblasts) could fuel the growth of adjacent epithelial cancer cells. Ketone producing enzymes (in the fibroblasts) and ketone re-utilizing enzymes (in the epithelial cancer cells) are shown in BOLD. Transfer of ketones would be accomplished by monocarboxylate transporters (MCTs). Normally the same scheme is used by the liver (for ketone production) and the brain (for ketone reutilization) during extreme fasting or starvation, to maintain neuronal function. Thus, the liver cells are the cancer fibroblasts, and the epithelial cells are the neurons. Interestingly, Cav-1 (−/−) stromal cells and the tumor stroma both show a shift towards liver-specific gene and protein expression. For example, Cav-1 (−/−) stromal cells produce alpha-fetoprotein and albumin, as seen by proteomics. Alpha-fetoprotein expression has been previously localized to cancer-associated fibroblasts in human breast cancers. The enzymes involved in ketone metabolism are as follows: ACYL, ATP citrate lyase (cytosolic); HMGCS1/2, 3-hydroxy-3-methylglutaryl-Coenzyme A synthase 1 (cytosolic)/2 (mitochondrial); HMGCL, 3-hydroxymethyl-3-methylglutaryl-Coenzyme A lyase; HMGCLL1, 3-hydroxymethyl-3-methylglutaryl-Coenzyme A lyase-like 1; BDH1/2, 3-hydroxybutyrate dehydrogenase, type 1 (mitochondrial)/type 2 (cytosolic); ACAT1/2, acetyl-Coenzyme A acetyltransferase 1 (mitochondrial)/2 (cytosolic); OXCT1/2, 3-oxoacid CoA transferase 1 (mitochondrial)/2 (testis-specific). The production of ketone bodies results from Acetyl-CoA derived from pyruvate, via pyruvate dehydrogenase (PDH), and not from the beta-oxidation of fatty acids, because Cav-1 (−/−) null mice have a defect in the beta-oxidation of fatty acids. This would also mechanistically explain why lactate does not accumulate. Interestingly, ACLY (a cytosolic enzyme) may also contribute to ketone production by converting citrate (a TCA metabolite) to Acetyl-CoA. This also results in the production of oxaloacetate, another TCA metabolite.

FIG. 9 shows Resolving the Autophagy Paradox in Cancer Therapy, Autophagy/mitophagy (AM) in the tumor stroma may be sustaining tumor growth. The large black arrow signifies energy transfer (E.T.) from the stromal cancer associated fibroblasts (CAFs) to the epithelial cancer cells, via stromal autophagy/mitophagy. Thus, inhibition of autophagy in the tumor stroma would be expected to halt or reverse tumor growth. This could explain the effectiveness of known autophagy inhibitors as anti-tumor agents 50, such as chloroquine and 3-methyladenine (Upper panel). Conversely, induction of autophagy in epithelial cancer cells would also be expected to block or inhibit tumor growth (Lower panel). This idea would also explain the anti-tumor activity of agents that activate autophagy, such as mTOR inhibitors. Thus, using this model, compounds that either systemically block or activate autophagy would both have the same net effect, which is to disrupt the metabolic coupling between the epithelial cancer cells and the tumor stromal fibroblasts. This model directly resolves the long-lived “autophagy paradox”, that both systemic inhibition of autophagy and systemic stimulation of autophagy have the same net effect, which is to inhibit tumor growth. E.T., energy transfer; AM+, increased autophagy/mitophagy; AM-, decreased autophagy/mitophagy; Rx, therapy with autophagy promoters or inhibitors.

FIG. 10 shows Ketones Promote Mammary Tumor Growth. A xenograft model was employed in which MDA-MB-231 breast cancer cells injected into the flanks of athymic nude mice to evaluate the potential tumor promoting properties of the products of aerobic glycolysis (such as 3-hydroxy-butyrate and L-lactate). Tumor growth was assessed by measuring tumor volume, at 3-weeks post tumor cell injection. During this time period, mice were administered either PBS alone, or PBS containing 3-hydroxy-butyrate (500 mg/kg) or L-lactate (500 mg/kg), via daily intra-peritoneal (i.p.) injections. Note that 3-hydroxy-butyrate is sufficient to promote an ˜2.5-fold increase in tumor growth, relative to the PBS-alone control. Under these conditions. L-lactate had no significant effect on tumor growth. *p<0.05, PBS alone versus 3-hydroxy-butyrate (Student's t-test).

FIG. 11 shows Ketones Promote Tumor Growth, Without Any Increase in Angiogenesis. Tumor angiogenesis could account for the tumor-promoting properties of 3-hydroxy-butyrate. Thus, the status of tumor vascularity was evaluated using antibodies directed against CD31. However, the vascular density (number of vessels per field) was not increased by the administration of either 3-hydroxy-butyrate or L-lactate. Thus, other mechanisms, such as the “Reverse Warburg Effect” may be operating to increase tumor growth. n.s., not significant.

FIG. 12 shows Ketones Promote Tumor Growth, Without Any Increase in Angiogenesis. Representative images of CD31 immuno-staining in primary tumor samples.

FIG. 13 shows Ketones and Lactate Function as Chemo-attractants, Stimulating Cancer Cell Migration. 3-hydroxy-butyrate or L-lactate can function as chemo-attractants. shown using a modified “Boyden Chamber” assay, employing Transwell cell culture inserts. MDA-MB-231 cells were placed in the upper chambers, and 3-hydroxy-butyrate (10 mM) or L-lactate (10 mM) were introduced into the lower chambers. Note that both 3-hydroxy-butyrate and L-lactate promoted cancer cell migration by nearly 2-fold. *p<0.05, control (vehicle alone) versus 3-hydroxy-butyrate or L-lactate (Student's t-test).

FIG. 14 shows Lactate Fuels Lung Metastasis. To examine the effect of 3-hydroxy-butyrate and L-lactate on cancer cell metastasis, a well-established lung colonization assay was used, where MDA-MB-231 cells are injected into the tail vein of athymic nude mice. After 7 weeks post-injection, the lungs were harvested and the metastases were visualized with India ink staining. Using this approach, the lung parenchyma stains black, while the tumor metastatic foci remain unstained, and appear white. The number of metastases per lung lobe was scored. Note that relative to PBS-alone, the administration of L-lacate stimulated the formation of metastatic foci by ˜10-fold. Under these conditions, 3-hydroxy-butryate had no effect on metastasis formation. *p<0.01, PBS alone versus L-lactate (Student's t-test and ANOVA); *p<0.05, PBS alone versus L-lactate (Mann-Whitney test).

FIG. 15 shows Lactate Fuels Lung Metastasis. Images of Lung Metastases. Representative examples of lung metastasis in PBS-alone controls and L-lactate-treated animals are shown. Note that the metastatic foci formed in L-lactate treated animals are more numerous, and are also larger in size.

FIG. 16 shows Cav-1 knock-down is sufficient to promote autophagy/mitophagy. Acute loss of Cav-1 increases the expression of autophagic markers. hTERT-fibroblasts were treated with Cav-1 siRNA or control (CTR) siRNA. Western blot analysis. Cells were analyzed by Western blot analysis using antibodies against the indicated autophagic markers. β-tubulin was used as equal loading control.

FIG. 17 shows Cav-1 knock-down is sufficient to promote autophagy/mitophagy. Immunofluorescence. Cells were fixed and immuno-stained with antibodies against beclin 1, BNIP3, and BNIP3L. DAPI was used to visualize nuclei. Importantly, paired images were acquired using identical exposure settings. Original magnification, 40×. Note that acute Cav-1 knockdown is sufficient to greatly increase the expression levels of all the autophagy/mitophagy markers examined.

FIG. 18 shows Human breast cancers lacking stromal Cav-1 display increased stromal BNIP3L. BNIP3L is highly increased in the stroma of human breast cancers that lack stromal Cav-1. Paraffin-embedded sections of human breast cancer samples lacking stromal Cav-1 were immuno-stained with antibodies directed against BNIP3L. Slides were then counter-stained with hematoxylin. Note that BNIP3L is highly expressed in the stromal compartment of human breast cancers that lack stroma Cav-1. The boxed area shown at higher magnification reveals punctate staining, consistent with mitochondrial and/or lysosomal localization. Original magnification, 40× and 60×, as indicated.

FIG. 19 shows the Reactions involved in Ketone Production (Ketogenesis).

FIG. 20 shows Reactions involved in Ketone (Re-)Utilization (Ketolysis).

FIGS. 21A through 21C showTable 1, Metabolomic Analysis of Mammary Fat Pads from Cav-1 (−/−) Deficient Mice.

FIG. 22 shows Table 2. Metabolomic Analysis of Mammary Fat Pads and Lung Tissue from Cav-1 (−/−) Deficient Mice.

FIGS. 23A and 23B show Table 3. Upregulation of Autophagy/Mitophagy Related Gene Transcripts in Cav-1 (−/−) Stromal Cells.

FIGS. 24A and 24B show Table 4. Upregulation of Gene Transcripts Encoding Lysosomal Proteins in Cav-1 (−/−) Stromal Cells.

FIGS. 25A through 25D show Table 5. Upregulation of Telomerase and Selected Redox-Related Gene Transcripts in Cav-1 (−/−) Stromal Cells.

FIGS. 26A and 26B show Table 6. Upregulation of Autophagy/Mitophagy Related Gene Transcripts in the Tumor Stroma from Human Breast Cancer Patients.

FIGS. 27A and 27B show Table 7. Upregulation of Gene Transcripts Encoding Lysosomal Proteins in the Tumor Stroma from Human Breast Cancer Patients.

FIGS. 28A and 28B show Table 8. Upregulation of Telomerase and Selected Redox-Related Gene Transcripts in the Tumor Stroma from Human Breast Cancer Patients.

FIGS. 29A and 29B show Table 9. Transcriptional Profiling of Human Breast Cancer Tumor Stroma: ADMA and BHB Metabolism.

FIG. 30 shows Table 10. Up-regulation of miR's in Cav-1 (−/−) null stromal cells.

FIGS. 31A through 31D show Table 11, Breast Cancer Epithelial Cells Show a Transcriptional Shift Towards Oxidative Mitochondrial Metabolism, Relative to Adjacent Stromal Tissue.

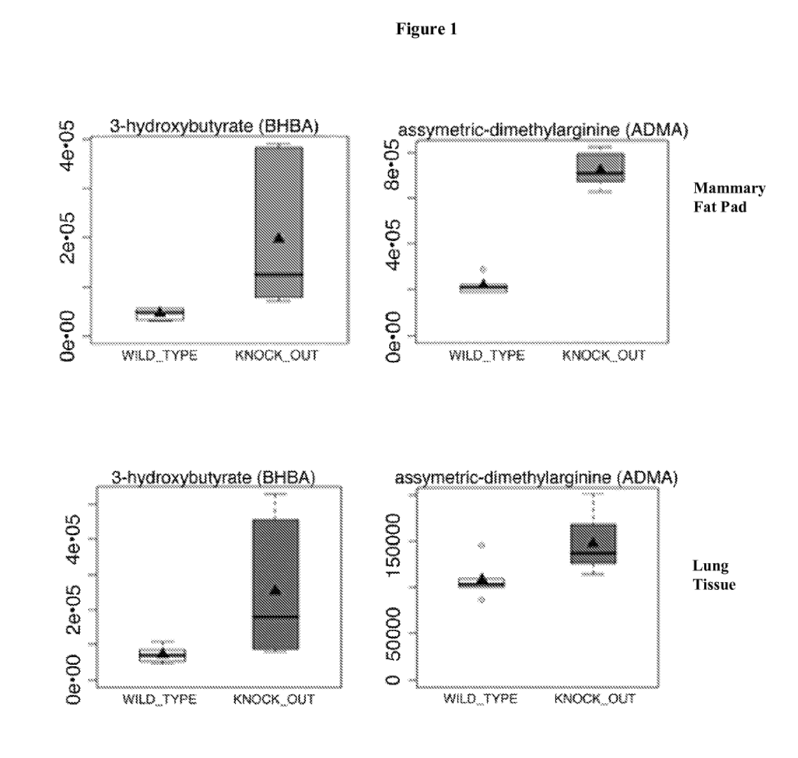

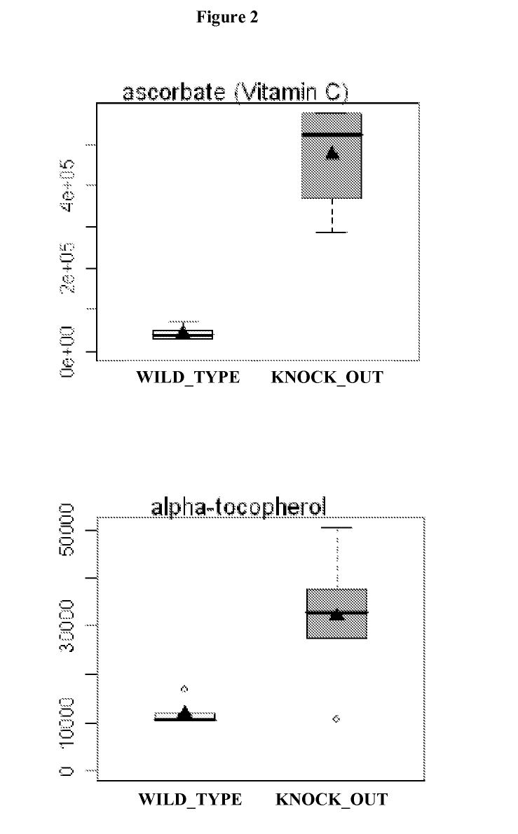

### DETAILED DESCRIPTION OF THE INVENTION

The mammary fat pad of Cav-1 (−/−) null mice as a pre-clinical model for a “lethal tumor-microenvironment”, i.e., the tumor stroma without the tumor. The inventors have previously documented that a loss of stromal Cav-1 in the fibroblast compartment of human breast cancer, DCIS, and prostate cancer is associated with a poor clinical outcome. In breast cancer, a loss of stromal Cav-1 is a single independent predictor of early tumor recurrence, lymph node metastasis, and tamoxifen-resistance. In DCIS, a loss of stromal Cav-1 predicts both early recurrence and progression to invasive breast cancer. Witkiewicz A K, Dasgupta A, Nguyen K H, Liu C, Kovatich A J, Schwartz G F, Pestell R G, Sotgia F, Rui H, Lisanti M P. Stromal caveolin-1 levels predict early DCIS progression to invasive breast cancer. Cancer Biol Ther 2009; 8:1167-75. Finally, in prostate cancer patients, a loss of stromal Cav-1 is associated with advanced prostate cancer and tumor progression/metastasis, and high Gleason score, indicative of a poor prognosis. Witkiewicz A K, Dasgupta A, Sotgia F, Mercier I, Pestell R G, Sabel M, Kleer C G, Brody J R, Lisanti M P. An Absence of Stromal Caveolin-1 Expression Predicts Early Tumor Recurrence and Poor Clinical Outcome in Human Breast Cancers. Am J Pathol 2009; 174:2023-34. Witkiewicz A K, Dasgupta A, Nguyen K H, Liu C, Kovatich A J, Schwartz G F, Pestell R G, Sotgia F, Rui H, Lisanti M P. Stromal caveolin-1 levels predict early DCIS progression to invasive breast cancer. Cancer Biol Ther 2009; 8:1167-75. Di Vizio D, Morello M, Sotgia F, Pestell R G, Freeman M R, Lisanti M P. An Absence of Stromal Caveolin-1 is Associated with Advanced Prostate Cancer, Metastatic Disease and Epithelial Akt Activation. Cell Cycle 2009; 8:2420-4. Witkiewicz A K, Casimiro M C, Dasgupta A, Mercier I, Wang C, Bonuccelli G, Jasmin J F, Frank P G, Pestell R G, Kleer C G, Sotgia F, Lisanti M P. Towards a new “stromal-based” classification system for human breast cancer prognosis and therapy. Cell Cycle 2009; 8:1654-8. Witkiewicz A K, Dasgupta A, Sammons S, Er O, Potoczek M B, Guiles F, Sotgia F, Brody J R, Mitchell E P, Lisanti M P. Loss of stromal caveolin-1 expression predicts poor clinical outcome in triple negative and basal-like breast cancers. Cancer Biol Ther 2010; 10: In Press.

### Oxidative Stress and Autophagy/Mitophagy in the Tumor Micro-Environment

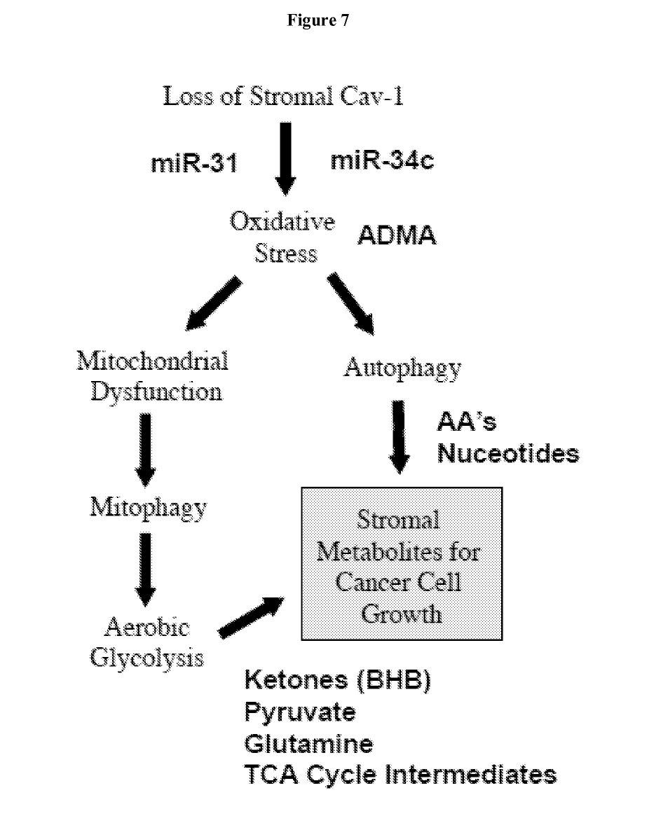

To mechanistically understand the lethality of a loss of Cav-1 in the tumor stromal compartment, an unbiased screening approach was used, by performing a metabolomics analysis on fresh tissue harvested from the mammary fat pads of Cav-1 (−/−) null mice, a robust animal model for a Cav-1 deficiency. Based on this analysis, the evidence for a series of severe metabolic defects in Cav-1 deficient tissues is provided. More specifically, the inventors show that nearly 100 metabolites are elevated in Cav-1 (−/−) null mammary fat pads. An analysis of these data is consistent with the onset of oxidative stress phenotype, combined with mitochondrial dysfunction, and autophagy. The two most significant metabolites that are elevated are ADMA and 3-hydroxybutyrate. Also, several energy-rich metabolites, such as pyruvate and metabolic components of the TCA cycle are increased. These phenotypic changes provide a logical and intriguing explanation for the lethality of a Cav-1 deficient tumor micro-environment, as oxidative stress is known to drive both mitochondrial dysfunction and autophagy/mitophagy, and this would a set-up a situation in which catabolism of the tumor stroma could be used to directly “feed” the tumor epithelial cancer cells. This is an exceptionally ingenious parasitic strategy that could promote tumor progression and metastasis (Summarized in FIG. 7).

To independently validate these assertions, an informatics approach to reinterrogate the transcriptional profiling data obtained from the analysis of Cav-1 (−/−) deficient stromal cells was used, isolated from the bone marrow of Cav-1 knockout mice. Importantly, bone marrow mesenchymal stem cells are thought to be the precursors of cancer associated fibroblasts that are recruited by epithelial tumor cells to cancerous lesions. Based on our re-analysis of this data set, the inventors provide evidence for the upregulation of numerous gene transcripts specifically associated with authophagy/mitophagy, lysosomal biogenesis, oxidative stress, the glutathione pathway, and the compensatory upregulation of anti-oxidant enzymes. These results provide direct independent validation of the metabolic profiling studies.

To directly assess the relevance of the findings for human breast cancers, evidence of the same transcriptional profiles in the tumor stromal that was laser capture micro-dissected from the primary human tumors of patients with breast cancer was examined. Importantly, re-interrogation of these data sets indicated that the following biological processes are well-represented in the tumor stroma: authophagy/mitophagy, lysosomal biogenesis, oxidative stress, the glutathione pathway, and the upregulation of anti-oxidant enzymes. Many of the transcripts associated with these processes were also related to tumor recurrence, and lymph-node metastasis.

### Identification of ADMA and Ketones as Key Metabolites: Implications for Diagnosis and Drug Discovery

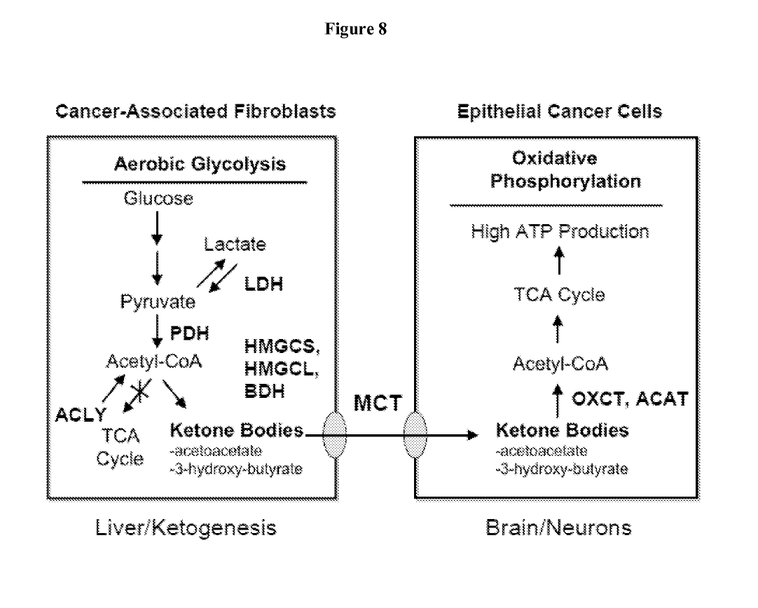

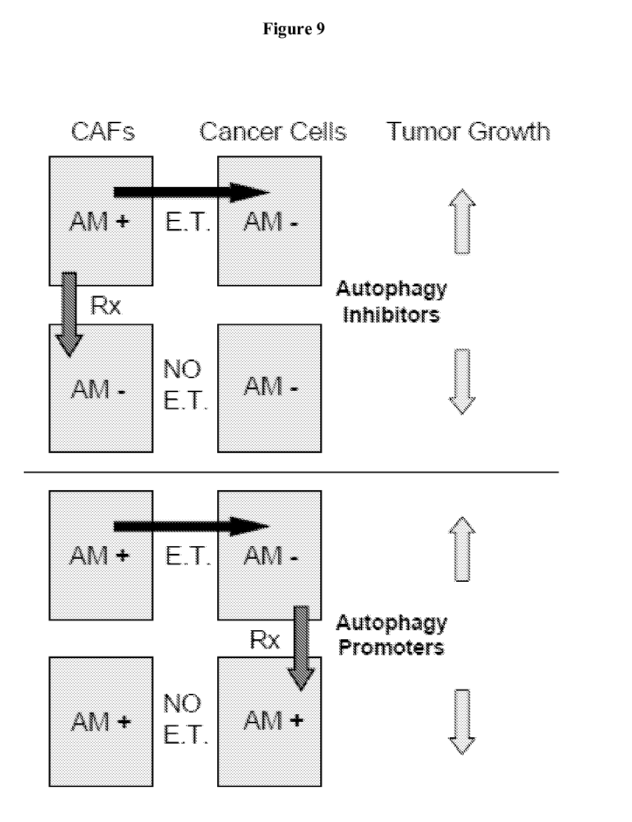

Since ADMA and 3-hydroxybutyrate emerged as the two most important metabolites that were increased in metabolomic analysis, the enzymes responsible for their production were transcriptionally increased both in Cav-1 (−/−) stromal cells and the tumor stroma isolated from human breast cancers was validated. Thus, these new observations now provide an opportunity for both diagnostic stratification of patients and the design of new drug therapies, to both identify and combat the lethality of an aggressive tumor microenvironment. In the case of ADMA, it is a catabolic breakdown product released from methylated proteins after their proteolytic degradation. It is known to be strongly associated with endothelial cell dysfunction and oxidative stress. In addition, it also has biologically activity and can enhance and propagate the effects of oxidative stress. For example, it is known to function as a natural endogenous inhibitor of nitric oxide synthase (NOS) enzymes, halting the production of nitric oxide (NO). However, it also changes the specificity of the NOS enzymes, allowing them to constitutively produce superoxide instead. Teerlink T, Luo Z, Palm F, Wilcox C S. Cellular ADMA: Regulation and action. Pharmacol Res 2009; 60:448-60. 20. Yildirim A O, Bulau P, Zakrzewicz D, Kitowska K E, Weissmann N, Grimminger F, Morty R E, Eickelberg O. Increased protein arginine methylation in chronic hypoxia: role of protein arginine methyltransferases. Am J Respir Cell Mol Biol 2006; 35:436-43. Sud N, Wells S M, Sharma S. Wiseman D A, Wilham J, Black S M. Asymmetric dimethylarginine inhibits HSP90 activity in pulmonary arterial endothelial cells: role of mitochondrial dysfunction. Am J Physiol Cell Physiol 2008; 294:C1407-18. Thus, ADMA is both a marker of oxidative stress and a producer of more oxidative stress. Furthermore, ADMA changes the location of eNOS and directly targets the enzyme to mitochodria, where it them produces superoxide. Thus, ADMA is a mitochondrial “time-bomb” that leads to irreversible oxidative damage within mitochondria, necessitating their destruction by mitophagy. This, in turn, provides a mechanism for turning on aerobic glycolysis, so that the stromal cells will produce energy to ensure their own survival. However, aerobic glycolysis in the stroma releases both lactate and pyruvate, that can be used by epithelial cancer cells undergoing TCA based oxidative metabolism, thereby providing paracrine energy for tumor growth. Stromal ketone production also likely plays a strong pathogenic role. Ketone production is a well-established marker of mitochondrial dysfunction. Kennaway N G, Buist N R, Darley-Usmar V M, Papadimitriou A, Dimauro S, Kelley R I, Capaldi R A, Blank N K, D'Agostino A. Lactic acidosis and mitochondrial myopathy associated with deficiency of several components of complex III of the respiratory chain. Pediatr Res 1984; 18:991-9. Robinson B H, McKay N, Goodyer P, Lancaster G. Defective intramitochondrial NADH oxidation in skin fibroblasts from an infant with fatal neonatal lacticacidemia. Am J Hum Genet 1985; 37:938-46, consistent with our assertions regarding ADMA, oxidative stress, and autophagy/mitophagy. Ketones are normally produced by the liver and virtually every other organ system in the body during periods of fasting and starvation, and they are then transferred to the brain to maintain survival of the organism. Just as pyruvate, and lactate can be secreted and taken up by monocarboxylic acid transporters (MCTs), the ketones 3-hydroxybutyrate and acetoacetate, both follow the same principles. Cahill G F, Jr., Veech R L. Ketoacids? Good medicine? Trans Am Clin Climatol Assoc 2003; 114:149-61; discussion 62-3. Veech R L. The therapeutic implications of ketone bodies: the effects of ketone bodies in pathological conditions: ketosis, ketogenic diet, redox states, insulin resistance, and mitochondrial metabolism. Prostaglandins Leukot Essent Fatty Acids 2004; 70:309-19. Veech R L, Chance B, Kashiwaya Y, Lardy H A, Cahill G F, Jr. Ketone bodies, potential therapeutic uses. IUBMB Life 2001; 51:241-7. So, ketone bodies can be transferred directly from stromal cancer-associated fibroblasts to epithelial cancer cells via MCTs, without any energy expenditure. Moreover, ketones are a “super-fuel” for mitochondria, producing more energy than lacate/pyruvate, and simultaneously decreasing oxygen consumption. In fact, because of these properties, ketones have been used to prevent ischemic tissue damage, in animal models undergoing either myocardial infarctions or stroke, leading to dramatically smaller ischemic/necrotic lesion area. So, just as ketones are a “super-fuel” under conditions of ischemia in the heart and in the brain, they could fulfill a similar function during tumorigenesis, as the tumor exceeds its blood supply. So, stromal ketone production could obviate the need for tumor angiogenesis. Once ketones are produced and released from stromal cells, they could then be re-utilized by epithelial cancer cells, where they could directly enter the TCA cycle, just like lactate and pyruvate. In this sense, ketones are a more powerful mitochondrial fuel, as compared with lactate and pyruvate. As a consequence, the “Reverse Warburg Effect” includes ketones as a paracrine energy source (Summarized in FIG. 8). In this scheme, the production of ketone bodies results from Acetyl-CoA derived from pyruvate, via pyruvate dehydrogenase (PDH), and not from the betaoxidation of fatty acids, because Cav-1 (−/−) null mice have a defect in the beta-oxidation of fatty acids (discussed within 16). This would mechanistically explain why lactate does not accumulate. Interestingly, ACLY (a cytosolic enzyme) may also contribute to ketone production by converting citrate (a TCA metabolite) to Acetyl-CoA.

Thus, ADMA and ketone bodies (3-hydroxybutyrate/acetoacetate) levels can be used as diagnostic tools to assess patient outcome. ADMA and ketone levels could either be measured in patient serum/plasma, or directly determined from homogenates of fresh tumor tissue. High AMDA and ketone levels in cancer patient serum or human tumor samples will strictly correlate with poor clinical outcome. These simple diagnostic tests could be performed rapidly, and quantitatively, allowing us to identify and monitor high-risk cancer patients, both at diagnosis and during therapy. They could also be used for treatment stratification.

There is also a new opportunity here for new drug development via targeted therapies. Inhibition of ADMA production or ketone production/re-utilization should halt tumor growth, leading to tumor regression. As such, the enzymes associated with i) ADMA production (all PRMT family members), ii) ketone production (ACYL, 18 HMGCS1/2, HMGCL, HMGCLL1, and BDH1/2) and iii) ketone re-utilization (ACAT1/2 and OXCT1/2) should now all be considered as “druggable targets” for cancer chemotherapy and prevention. In fact, a number of known anti-oxidants have already been shown to have anti-tumor activity 48, such as N-acetyl cysteine (NAC), vitamin C, quercetin, and curcumin. In this regard, NAC acts both as a free radical scavenger, and directly feeds into the glutathione pathway, increasing the amounts of cellular glutathione; NAC is the known to be the most promising anti-oxidant for inhibiting mitophagy. Deffieu M, Bhatia-Kissova I, Salin B, Galinier A, Manon S, Camougrand N. Glutathione participates in the regulation of mitophagy in yeast. J Biol Chem 2009; 284:14828-37. Furthermore, also anti-lysosomal drugs that inhibit autophagy, such as chloroquine, are known to have very significant anti-tumor activity. This may be due to their ability to inhibit autophagy in the fibroblastic stromal tumor compartment. Cancer Connections with Systemic Sclerosis, Diabetes, and Fasting Interestingly, a variety of human diseases are also associated with high levels of ADMA. One such disease is systemic sclerosis (Sec; scleroderma), and Sec patients have a higher incidence of cancer. Similarly, diabetic patients show both high serum levels of ADMA and ketones. Thus, our current observations may also explain the close and emerging association between diabetes and cancer susceptibility 60. A number of elegant studies have been carried out in mouse animal models to assess this association, and chemical induction of diabetes in rats with streptozocin is sufficient to enhance tumor growth 61. Similarly, acute fasting in rodent animal models is also sufficient to dramatically increase tumor growth. Both of these experimental conditions (diabetes and fasting/starvation) are known to be highly ketogenic, and, thus, are consistent with our current hypothesis that ketone production fuels tumor growth and metastasis. Thus, the combination of ADMA and ketones plays a crucial and causal role in promoting tumorigenesis, by providing oxidative stress and the simultaneous release of high-energy nutrients from the tumor micro-environment. Of course, this would be complemented by oxidative stress induced autophagy/mitophagy in the tumor microenvironment, thus providing the necessary recycled chemical building blocks (amino acids, nucleotides, TCA cycle intermediates, etc.) in a paracrine fashion to cancer epithelial cells, to promote tumor growth. Hu C, Solomon V R, Ulibarri G, Lee H. The efficacy and selectivity of tumor cell killing by Akt inhibitors are substantially increased by chloroquine. Bioorg Med Chem 2008; 16:7888-93. Hiraki K, Kimura I. Studies on the Treatment of Malignant Tumors with Fibroblast-Inhibiting Agent. 1. Fibroblast-Inhibiting Action of Chloroquine. Acta Med Okayama 1963; 17:231-8. Hiraki K, Kimura I. Studies on the Treatment of Malignant Tumors with Fibroblast-Inhibiting Agent. Ii. Effects of Chloroquine on Animal Tumors. Acta Med Okayama 1963; 17:239-52. Hiraki K, Kimura I. Studies on the Treatment of Malignant Tumors with Fibroblast-Inhibiting Agent. Iv. Effects of Chloroquine on Malignant Lymphomas. Acta Med Okayama 1964; 18:87-92. Hiraki K, Kimura I. Studies on the Treatment of Malignant Tumors with Fibroblast-Inhibiting Agent. 3. Effects of Chloroquine on Human Cancers. Acta Med Okayama 1964; 18:71-85. Dooley A, Gao B, Bradley N, Abraham D J, Black C M, Jacobs M, Bruckdorfer K R. Abnormal nitric oxide metabolism in systemic sclerosis: increased levels of nitratedproteins and asymmetric dimethylarginine. Rheumatology (Oxford) 2006; 45:676-84. Dimitroulas T, Giannakoulas G, Sfetsios T, Karvounis H, Dimitroula H, Koliakos G, Settas L. Asymmetrical dimethylarginine in systemic sclerosis-related pulmonary arterial hypertension. Rheumatology (Oxford) 2008; 47:1682-5. Marasini B, Conciato L, Belloli L, Massarotti M. Systemic sclerosis and cancer. Int J Immunopathol Pharmacol 2009; 22:573-8. Anderssohn M, Schwedhelm E, Luneburg N, Vasan R S, Boger R H. Asymmetric dimethylarginine as a mediator of vascular dysfunction and a marker of cardiovascular disease and mortality: an intriguing interaction with diabetes mellitus. Diab Vasc Dis Res 2010; 7:105-18. Pitocco D, Zaccardi F, Di Stasio E, Romitelli F, Martini F, Scaglione G L, Speranza D, Santini S, Zuppi C, Ghirlanda G. Role of asymmetric-dimethyl-L-arginine (ADMA) and nitrite/nitrate (NOx) in the pathogenesis of oxidative stress in female subjects with uncomplicated type 1 diabetes mellitus. Diabetes Res Clin Pract 2009; 86:173-6. Nicolucci A. Epidemiological aspects of neoplasms in diabetes. Acta Diabetol 2010; 47:87-95.

In support of these ideas linking fasting/autophagy, with cancer susceptibility and diabetes, adipocytes from obese patients with type 2 diabetes show decreased mTOR signaling and substantially enhanced autophagy. Similarly, hypoxia, inflammation, and michondrial dysfunction all inactivate mTOR signaling, leading to autophagy. Ost A, Svensson K, Ruishalme I, Brannmark C, Franck N, Krook H, Sandstrom P, Kjolhede P, Stralfors P. Attenuated mTOR signaling and enhanced autophagy in adipocytes from obese patients with type 2 diabetes. Mol Med 2010.

### Autophagy in the Tumor Micro-Environment can Substitute for Angiogenesis in Promoting Tumor Growth

The combination of oxidative stress, mitochondrial dysfunction, and autophagy/mitophagy in cancer-associated fibroblasts reduces the dependence of tumor growth and survival on neo-angiogenesis and vascularization. This explains why many of the new angiogenesis inhibitors have not been as promising as expected in ongoing clinical trials, as our current observations suggest that a Cav-1 negative fibroblastic tumor micro-environment could actually subsume the role of tumor angiogenesis, without the need for increased tumor vascularization. This is particularly relevant in the case of pancreatic cancers, which are known to be a highly fibrotic class of relatively avascular tumors which are exceptionally lethal. Genetically modified glycolytic fibroblasts, that lack Cav-1 expression, were used to assess their affects on human xenograft tumor growth using co-injections with a breast cancer cell line, namely MDA-MB-231 cells. In these xenograft models, the genetically modified fibroblasts, lacking Cav-1 expression, increased tumor weight by ˜4-fold, and tumor volume by nearly 8-fold, without a measurable increase in tumor angiogenesis. Migneco G, Whitaker-Menezes D, Chiavarina B, Castello-Cros R, Pavlides S, Pestell R G, Fatatis A, Flomenberg N, Tsirigos A, Howell A, Martinez-Outschoom U E, Sotgia F, Lisanti M P. Glycolytic cancer associated fibroblasts promote breast cancer tumor growth, without a measurable increase in angiogenesis: Evidence for stromalepithelial metabolic coupling. Cell Cycle 2010; 9.

Micro-RNA Profiling: Associations with Oxidative Stress and Autophagy/Mitophagy.

Micro-RNA (miR) profiling on Cav-1 (−/−) deficient stromal cells was performed to gain mechanistic insight into how a loss of Cav-1 may drive oxidative stress, mitochondrial dysfunction, and autophagy/mitophagy. Using this approach, 20 two miR species were identified that were highly over-expressed in Cav-1 null stromal cells, namely miR-31 and miR-34c.

The upregulation of miR-34c is consistent with results from both metabolomics and transcriptional profiling, as it is normally upregulated by oxidative stress, and is also associated with DNA damage and senescence, which are known down-stream effects of oxidative stress. Similarly, the upregulation of miR-31 provides a means for the transcriptional activation of HIF1-alpha 18, which is known to induce both autophagy, and mitophagy, and to inhibit mitochondrial biogenesis. The transcriptional activation of HIF1-alpha by miR-31 is indirectly mediated by FIH-1 (factor inhibiting HIF), which is the direct target of miR-31 18. Thus, over-expression of miR-31 blocks the transcriptional expression of a HIF inhibitory factor, FIH-1, leading to HIF activation. The Autophagic Tumor Stroma Model of Cancer: Compartmentalized Autophagy. Based on current observations, the inventors have developed a new model for cancer pathogenesis. In this model, tumor cells activate autophagy in the tumor stromal compartment via paracrine mechanisms. Autophagy in the tumor stroma, especially in cancer-associated fibroblasts, then provides epithelial cancer cells with a steady stream of recycled nutrients and energy-rich metabolites which could then be re-used by cancer cells to drive increases in tumor growth and metastasis. Additional mesenchymal stem cells from the bone marrow can be recruited to the tumor and induced to undergo autophagy, to satisfy the tumor's appetite. The extension of this scheme from a local to a systemic phenomenon, can explain the onset of anorexia, cachexia, insulin-resistance, and metabolic syndrome, all features that are known to be associated with chronic malignancy, and this would provide the tumor with autophagic/catabolic-based nutrients (including ketone bodies)—from distant systemic sources. Tumor cells might even metastasize to the major sites of ketone production (the liver or adipose-tissue-rich bone marrow) or ketone re-utilization (the brain), in search of energy-rich metabolites. Cannell I G, Kong Y W, Johnston S J, Chen M L, Collins H M, Dobbyn H C, Elia A, Kress T R, Dickens M, Clemens M J, Heery D M, Gaestel M, Eilers M, Willis A E, Bushell M. p38 MAPK/MK2-mediated induction of miR-34c following DNA damage prevents Myc-dependent DNA replication. Proc Natl Acad Sci USA 2010; 107:5375-80. He X, He L, Hannon G J. The guardian's little helper: microRNAs in the p53 tumor suppressor network. Cancer Res 2007; 67:11099-101. Lafferty-Whyte K, Cairney C J, Jamieson N B, Oien K A, Keith W N. Pathway analysis of senescence-associated miRNA targets reveals common processes to different senescence induction mechanisms. Biochim Biophys Acta 2009; 1792:341-52. Mazure N M, Pouyssegur J. Hypoxia-induced autophagy: cell death or cell survival? Curr Opin Cell Biol 2010; 22:177-80. Bellot G, Garcia-Medina R, Gounon P, Chiche J, Roux D, Pouyssegur J, Mazure N M. Hypoxia-induced autophagy is mediated through hypoxia-inducible factor induction of BNIP3 and BNIP3L via their BH3 domains. Mol Cell Biol 2009; 29:2570-81. Chan S Y, Loscalzo J. MicroRNA-210: A unique and pleiotropic hypoxamir. Cell Cycle 2010; 9. 39. Liu C J, Kao S Y, Tu H F, Tsai M M, Chang K W, Lin S C. Increase of microRNA miR-31 level in plasma could be a potential marker of oral cancer. Oral Dis 2010. Wang C J, Zhou Z G, Wang L, Yang L, Zhou B, Gu J, Chen H Y, Sun X F. Clinicopathological significance of microRNA-31, -143 and -145 expression in colorectal cancer. Dis Markers 2009; 26:27-34.

This model also provides a rationale basis for designing new therapeutic intervention(s), as autophagy in the tumor stroma may be sustaining tumor growth. Thus, inhibition of autophagy in the tumor stroma halts or reverses tumor growth. This can explain the effectiveness of known autophagy inhibitors as anti-tumor agents, such as chloroquine and 3-m ethyladenine. Conversely, induction of autophagy in epithelial cancer cells would also be expected to block or inhibit tumor growth. This idea would also explain the anti-tumor activity of agents that activate autophagy, such as mTOR inhibitors. Thus, using this model, compounds that either systemically block or activate autophagy would both have the same net effect, which is to disrupt the metabolic coupling between the epithelial cancer cells and the tumor stromal fibroblasts (Figure ______). This model directly resolves the long-lived “autophagy paradox”, that both systemic inhibition of autophagy and systemic stimulation of autophagy have the same net effect, which is to inhibit tumor growth. This new model provides a new paradigm and rationale basis for drug development, driving new metabolic therapeutic interventions.

Clinical Connections with Malignancy: ADMA, ATG16L, and SPARC

The inventors have found an association of ADMA and ketone production with malignancy, based on metabolomics analysis of a mouse model of a “lethal tumor-microenvironment”. In addition, the inventors have elucidated the role of autophagy in the tumor micro-environment. With regard to An autophagic marker, ATG16L, high levels of stromal ATG16L were associated with i) the lympho-vascular invasion of tumor cells and ii) positive lymph node status—consistent with our proposed model. Unfortunately, no data on clinical outcome were presented. The inventors have found that ATG16L was transcriptionally over-expressed in Cav-1 (−/−) stromal cells and the tumor stroma of human breast cancer patients, and its expression was associated with tumor recurrence (See Tables 3 and 6, Figures ______ and ______). Thus, high expression of autophagy-associated biomarkers in the tumor stroma are a general feature of human epithelial cancers and are associated with poor clinical outcome. SPARC is a multi-functional extracellular matrix protein that is associated with the tumor stroma. Recently, SPARC over-expression has been shown to be sufficient to induce autophagy in cells in culture via the up-regulation of cathepsin B. Similarly, the inventors have previously demonstrated that Cav-1 (−/−) deficient stromal cells over-express SPARC, as evidenced by both unbiased proteomic and genome-wide transcriptional profiling. Also, it was shown that the stromal expression of SPARC accurately predicts DCIS recurrence and/or progression. Taken together, these finding are consistent with the idea that a loss of stromal Cav-1 induces SPARC and autophagy in the tumor microenvironment, thereby promoting tumor progression in DCIS patients. In accordance with this idea, a loss of stromal Cav-1 is strongly associated with progression to invasive breast cancer in DCIS patients.

The inventors provide the first evidence that the end-products of aerobic glycolysis (namely, 3-hydroxy-butyrate and L-lactate) can fuel tumor growth and metastasis, when administered systemically in a human tumor xenograft model. More specifically, 3-hydroxy-butyrate is sufficient to promote a 2.5-fold increase in tumor volume, without any significant increases in angiogenesis. Although L-lactate did not increase tumor growth, it had a significant effect on lung colonization/metastasis, resulting in a 10-fold increase in the formation of metastatic tumor foci. The results are consistent with the idea that human breast cancer cells can re-utilize the energy-rich end-products of glycolysis for oxidative mitochondrial metabolism. Consistent with these functional xenograft data, the inventors also show, oxidative mitochondrial metabolism is indeed up-regulated in human breast cancer cells, relative to adjacent stromal tissue, measured using a transcriptional informatics analysis.

### Ketones and Tumor Growth: the Diabetes-Cancer Connection.

Ketones are a “super-fuel” for mitochondria, producing more energy than lacate, and simultaneously decreasing oxygen consumption. In fact, because of these properties, ketones have been used to prevent ischemic tissue damage, in animal models undergoing either myocardial infarctions or stroke, leading to dramatically smaller ischemic/necrotic lesion area. So, just as ketones are a “super-fuel” under conditions of ischemia in the heart and in the brain, the inventors found they fill a similar function during tumorigenesis, as the hypoxic tumor exceeds its blood supply. Stromal ketone production obviates the need for tumor angiogenesis. Once ketones are produced and released from stromal cells, they could then be re-utilized by epithelial cancer cells, where they could directly enter the TCA cycle, just like lactate. In this sense, ketones are a more powerful mitochondrial fuel, as compared with lactate. Cahill G F, Jr., Veech R L. Ketoacids? Good medicine? Trans Am Clin Climatol Assoc 2003; 114:149-61; discussion 62-3. Veech R L. The therapeutic implications of ketone bodies: the effects of ketone bodies in pathological conditions: ketosis, ketogenic diet, redox states, insulin resistance, and mitochondrial metabolism. Prostaglandins Leukot Essent Fatty Acids 2004; 70:309-19. Veech R L, Chance 13, Kashiwaya Y, Lardy H A, Cahill G F, Jr. Ketone bodies, potential therapeutic uses. IUBMB Life 2001; 51:241-7. Zou Z, Sasaguri S, Rajesh K G, Suzuki R. dl-3-Hydroxybutyrate administration prevents myocardial damage after coronary occlusion in rat hearts. Am J Physiol Heart Circ Physiol 2002; 283:H1968-74. Puchowicz M A, Zechel J L, Valerio J, Emancipator D S, Xu K, Pundik S, LaManna J C, Lust W D. Neuroprotection in diet-induced ketotic rat brain after focal ischemia. J Cereb Blood Flow Metab 2008; 28:1907-16.

Thus, our current observations may also explain the close and emerging association between diabetes and cancer susceptibility. A number of elegant studies have been carried out in mouse animal models to assess this association, and chemical induction of diabetes in rats with streptozocin is sufficient to enhance tumor growth. Similarly, acute fasting in rodent animal models is also sufficient to dramatically increase tumor growth. Both of these experimental conditions (diabetes and fasting/starvation) are known to be highly ketogenic, and, thus, are consistent with the model wherein ketone production fuels tumor growth. Finally, given our current findings that ketones increase tumor growth, cancer patients and their dieticians may want to re-consider the use of a “ketogenic diet” as a form of anti-cancer therapy. Nicolucci A. Epidemiological aspects of neoplasms in diabetes. Acta Diabetol 2010; 47:87-95. Sauer L A, Dauchy R T. Stimulation of tumor growth in adult rats in vivo during acute streptozotocin-induced diabetes. Cancer Res 1987; 47:1756-61. Goodstein M L, Richtsmeier W J, Sauer L A. The effect of an acute fast on human head and neck carcinoma xenograft. Growth effects on an ‘isolated tumor vascular pedicle’ in the nude rat. Arch Otolaryngol Head Neck Surg 1993; 119:897-902.

### Lactate Drives Metastatic Disease Progression: Quercetin and Lactated Ringers Solution.

Tumor lactate production, serum lactate levels, and serum LDH levels have long been known as biomarkers for poor clinical outcome in many different types of human epithelial cancers, including breast cancer. In fact, lactic-acidosis (due to the over-production and/or accumulation of serum lactate) is often the cause of death in patients with metastatic breast cancer, or other types of metastatic cancer 34-49. However, a causative role for L-lactate production in tumor metastatic progression has not yet been suggested or demonstrated. Koukourakis M I, Kontomanolis E, Giatromanolaki A, Sivridis E, Liberis V. Serum and tissue LDH levels in patients with breast/gynaecological cancer and benign diseases. Gynecol Obstet Invest 2009; 67:162-8. Ryberg M, Nielsen D, Osterlind K, Andersen P K, Skovsgaard T, Dombernowsky P. Predictors of central nervous system metastasis in patients with metastatic breast cancer. A competing risk analysis of 579 patients treated with epirubicin-based chemotherapy. Breast Cancer Res Treat 2005; 91:217-25. Nisman B, Barak V, Hubert A, Kaduri L, Lyass O, Baras M, Peretz T. Prognostic factors for survival in metastatic breast cancer during first-line paclitaxel chemotherapy. Anticancer Res 2003; 23:1939-42. Ryberg M, Nielsen D, Osterlind K, Skovsgaard T, Dombernowsky P. Prognostic factors and long-teen survival in 585 patients with metastatic breast cancer treated with epirubicin-based chemotherapy. Ann Oncol 2001; 12:81-7. Vigano A, Bruera E, Jhangri G S, Newman S C, Fields A L, Suarez-Almazor M E. Clinical survival predictors in patients with advanced cancer. Arch Intern Med 2000; 160:861-8. Kher A, Moghe G. Deshpande A. Significance of serum ferritin and lactate dehydrogenase in benign and malignant disease of breast. Indian J Pathol Microbiol 1997; 40:321-6. Khan N, Tyagi S P, Salahuddin A. Diagnostic and prognostic significance of serum cholinesterase and lactate dehydrogenase in breast cancer. Indian J Pathol Microbiol 1991; 34:126-30. Yeshowardhana, Gupta M. M, Bansal G, Goyal S, Singh V S, Jain S. Sangita Jain K. Serum glycolytic enzymes in breast carcinoma. Tumori 1986; 72:35-41.

Here, the inventors have directly demonstrated that L-lactate can play a causative role in breast cancer cell metastasis, by increasing the number of lung metastatic foci by ˜10-fold. This provides the necessary evidence that mitochondrial oxidative metabolism can also fuel cancer cell metastasis. This may have important clinical implications, as MCT/lactate transport inhibitors could be used to therapeutically to suppress tumor metastasis. Our findings also explain the multiple therapeutic activities of quercetin. Quercetin is a naturally occurring dietary flavenoid (available as an over-the-counter supplement) that functions both as an MCT/lactate transport inhibitor, and inhibitor of TGF-beta signaling. One explanation for these dual activities is that L-lactate uptake into tumor cells somehow metabolically activates TGF-beta signaling. As such, the inhibitory effects of quercetin on TGF-beta signaling may be due to its ability to inhibit the uptake of L-lactate into tumor cells, presumably resulting in reduced cell migration and metastasis. Further studies will be necessary to address this attractive possibility. Belt J A, Thomas J A, Buchsbaum R N, Racker E. Inhibition of lactate transport and glycolysis in Ehrlich ascites tumor cells by bioflavonoids. Biochemistry 1979; 18:3506-11. Hu Q, Noor M, Wong Y F, Hylands P J, Simmonds M S, Xu Q, Jiang D, Hendry B M. In vitro anti-fibrotic activities of herbal compounds and herbs. Nephrol Dial Transplant 2009. Phan T T, Lim I J, Chan S Y, Tan E K, Lee S T, Longaker M T. Suppression of transforming growth factor beta/smad signaling in keloid-derived fibroblasts by quercetin: implications for the treatment of excessive scars. J Trauma 2004; 57:1032-7. Subramanian A, Tamayo P, Mootha V K, Mukherjee S, Ebert B L, Gillette M A, Paulovich A, Pomeroy S L, Golub T R, Lander E S, Mesirov J P. Gene set enrichment analysis: a knowledge-based approach for interpreting genome-wide expression profiles. Proc Natl Acad Sci USA 2005; 102:15545-50.

Finally, given the pro-metastatic activity of L-lactate, its medical use in cancer patients should be restricted. However, nearly every oncology surgeon world-wide uses “Lactated Ringers” (which contains 25 mM L-lactate) as an intravenous (i.v.) solution in cancer patients, before, during, and after tumor excision, and possibly during the entire extended post-operative hospital stay. Based on our current studies, the use of “Lactated Ringers” in cancer patients may unnecessarily increase their risk for progression to metastatic disease. Thus, oncology surgeons may wish to re-consider using “Lactated Ringers” solution in cancer patients.

### Loss of Cav-1 is Sufficient to Induce Autophagy.

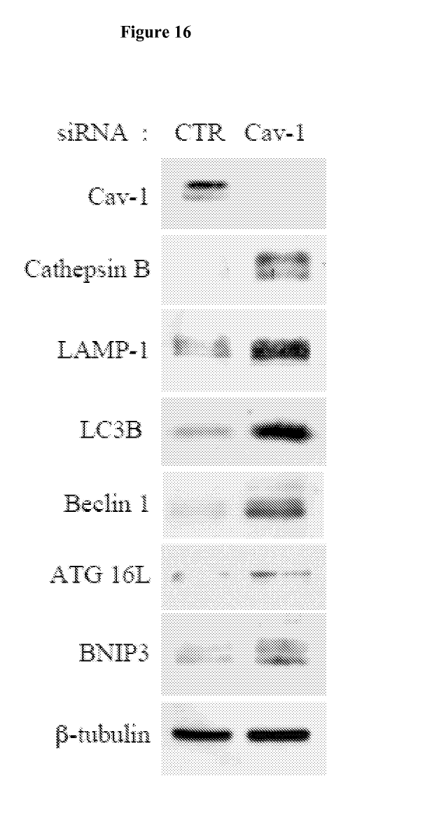

Loss of Cav-1 is sufficient to activate the autophagy and/or mitophagy program in fibroblasts. Western blot analysis was performed on hTERT-fibroblasts treated with Cav-1 siRNA or control siRNA, using antibodies directed against a panel of autophagy markers. FIG. 16 shows that acute Cav-1 knock-down in fibroblasts drives the increased expression of six autophagy markers, including Cathepsin B (active form), LAMP-1, LC3B, Beclin 1, ATG16L, and BNIP3.

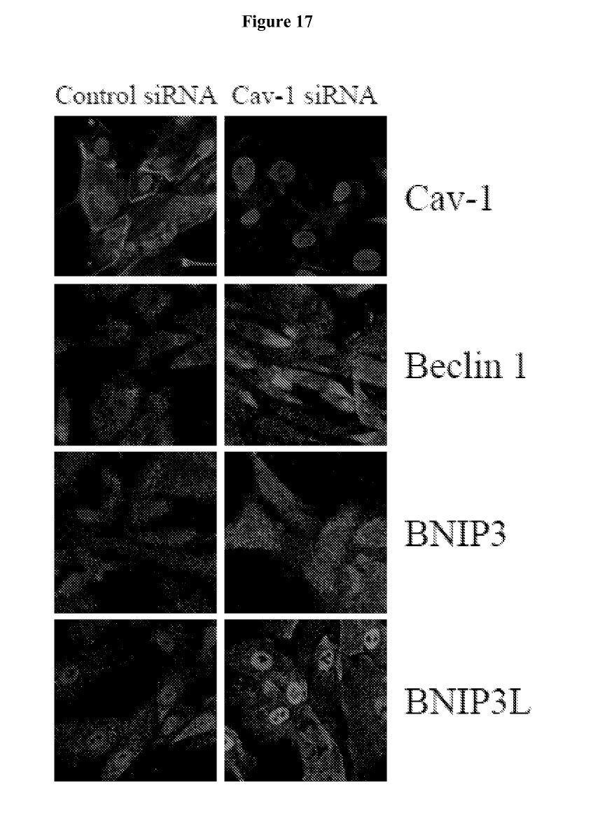

To independently validate these results, hTERT-fibroblasts treated with Cav-1 siRNA or control siRNA were also immuno-stained with a subset of autophagy/mitophagy markers. FIG. 17 shows that Beclin 1, BNIP3 and BNIP3L are greatly increased in Cav-1 knock-down cells, indicating that an acute loss of Cav-1 is sufficient to promote autophagy. Taken together, our current findings indicate that oxidative stress and hypoxia induce the autophagy-mediated loss of Cav-1 in fibroblasts, and that loss of Cav-1 further promotes autophagy/mitophagy in fibroblasts, via a feed-forward mechanism.

### Human Breast Cancers Lacking Stromal Cav-1 Display Increased Stromal BNIP3L.

The inventors have shown that a loss of Cav-1 promotes autophagy and that Cav-1 is degraded via an autophagic mechanism. To evaluate the translational significance of our findings, loss of stromal Cav-1 in human breast cancer was evaluated and correlates with increased autophagy. To this end, a number of human breast cancer samples were selected that lack Cav-1 in the stroma to perform immuno-staining with the autophagy/mitophagy marker BNIP3L.

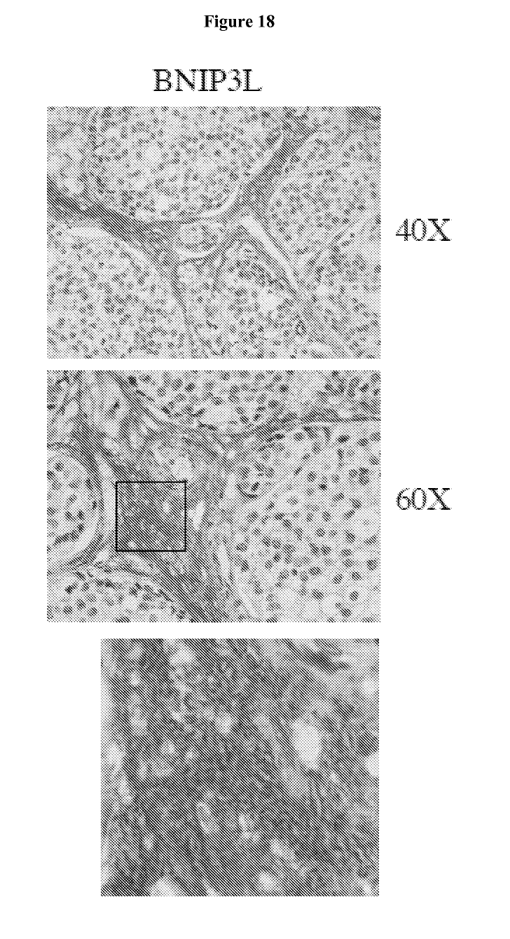

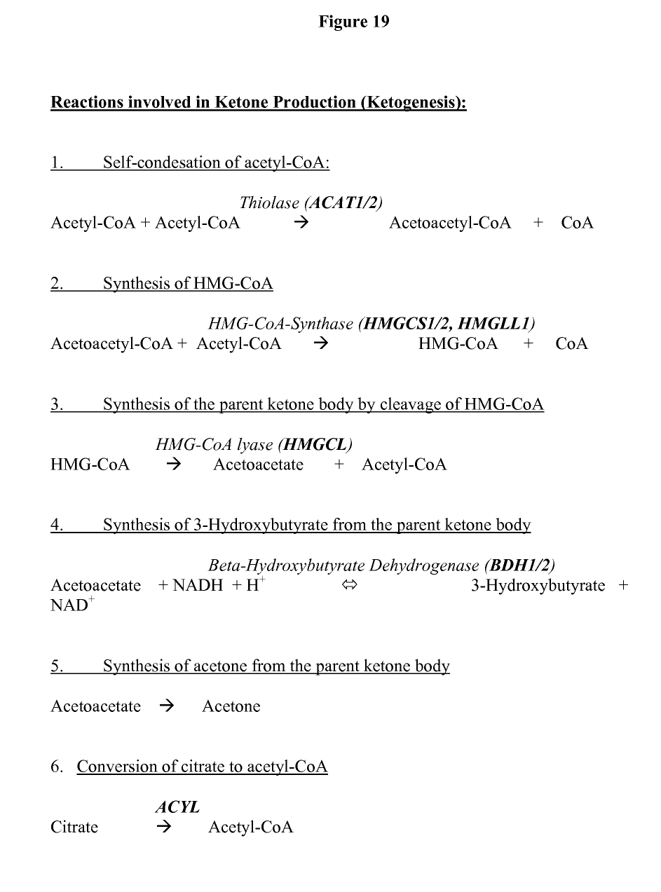

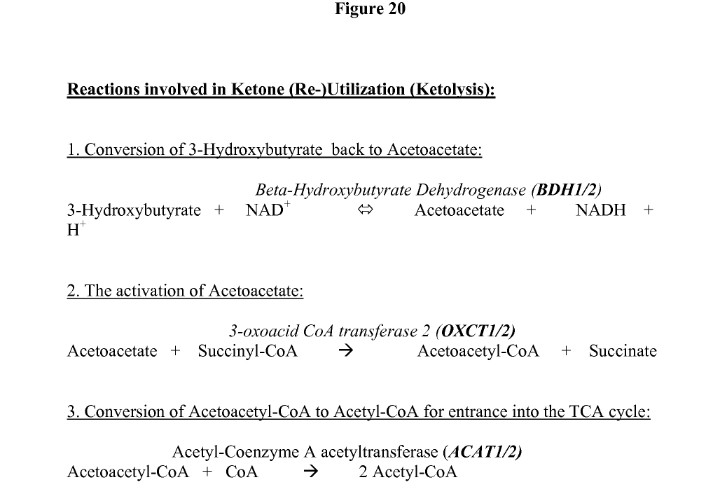

FIG. 18 shows that BNIP3L is highly expressed in the stromal compartment of human breast cancers that lack Cav-1. These results directly support the “Autophagic Tumor Stroma Model of Cancer Metabolism”. As a loss of Cav-1 is a powerful predictor of poor clinical outcome in breast cancers, our findings indicate that in human breast cancer a loss of Cav-1 promotes autophagy/mitophagy in the stroma, to support the growth and aggressive behavior of adjacent cancer cells.

Biomarkers

The present invention relates to biomarkers that are differentially expressed in neoplastic disease compared to normal patients, and various methods, reagents and kits for diagnosis, staging, prognosis, monitoring and treatment of neoplastic disease, including, e.g., breast cancer.

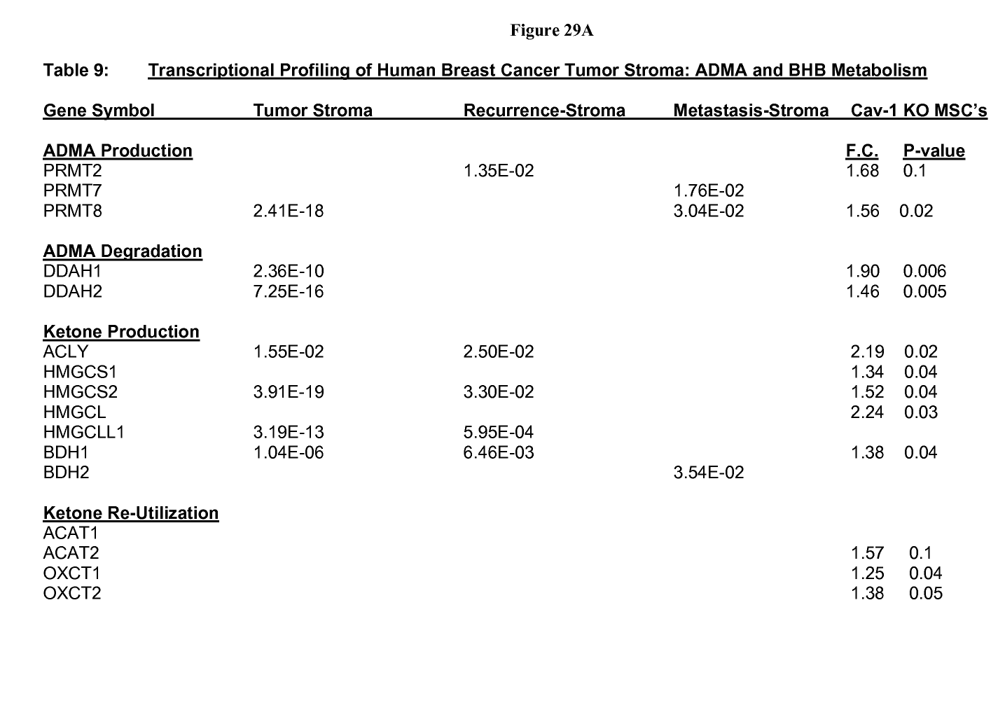

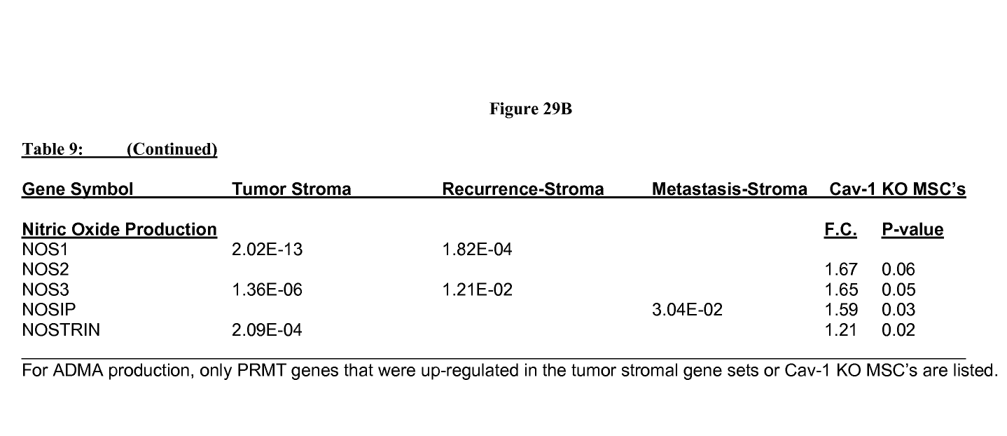

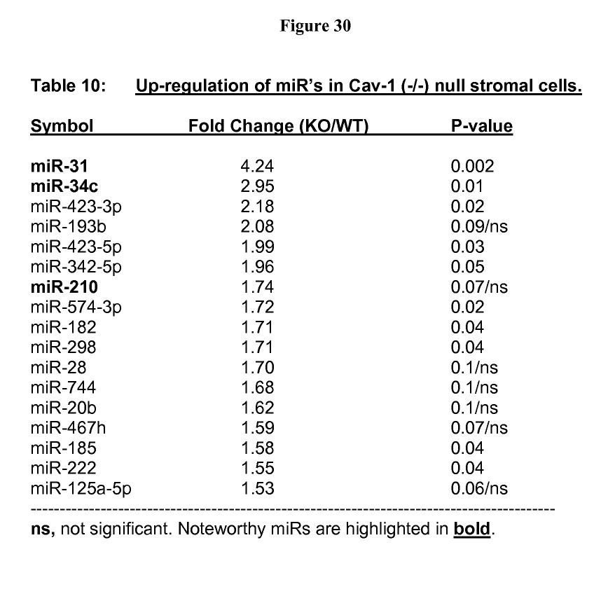

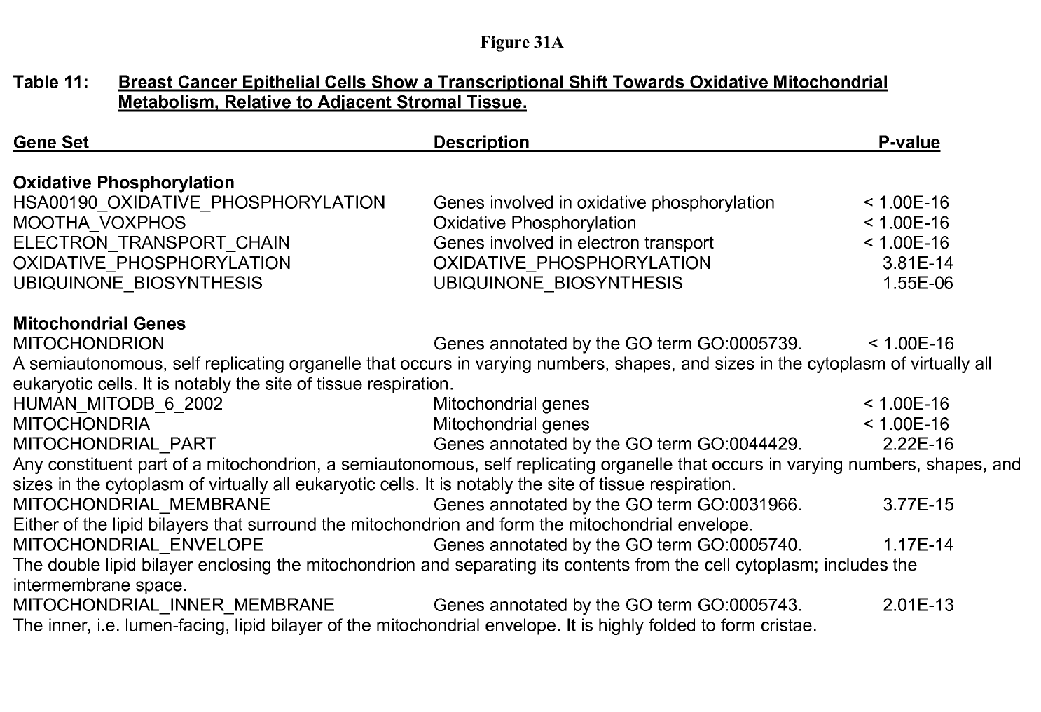

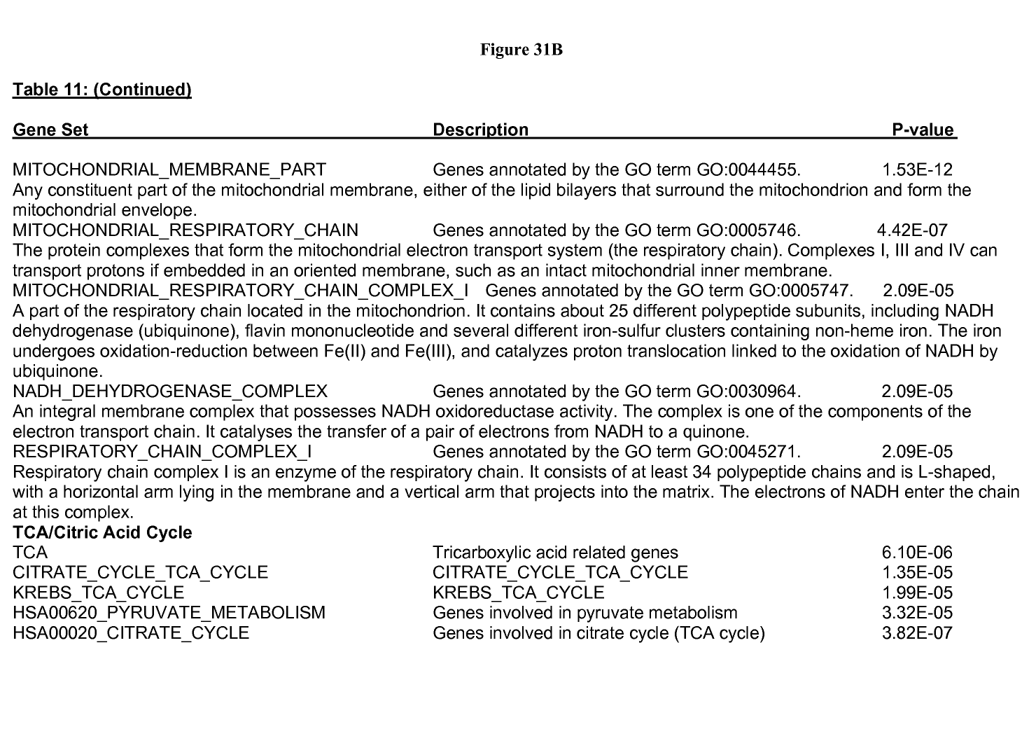

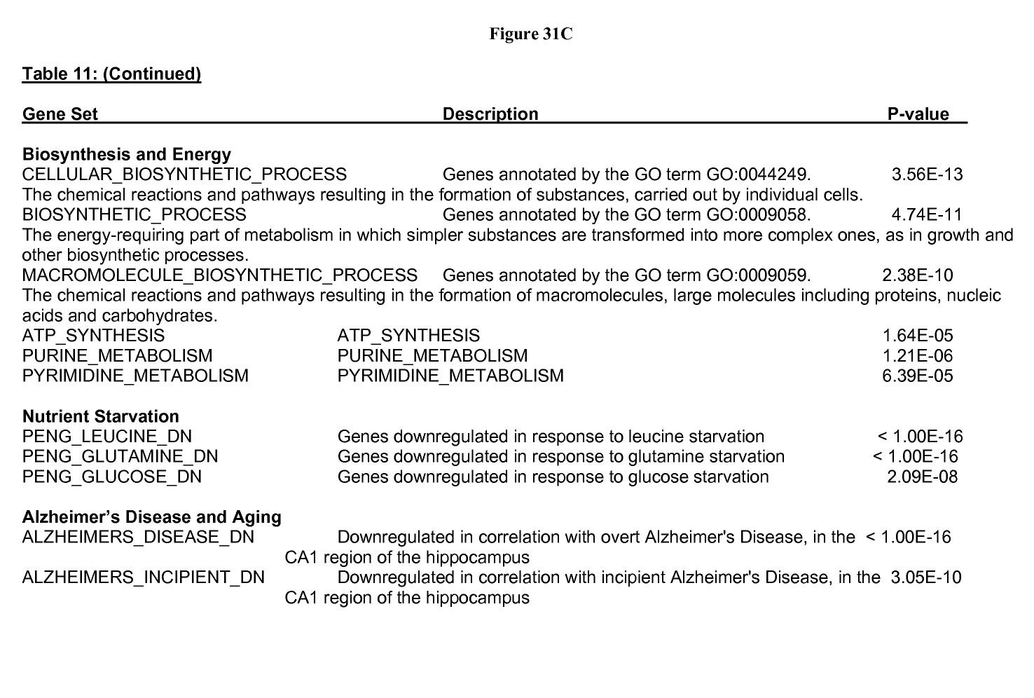

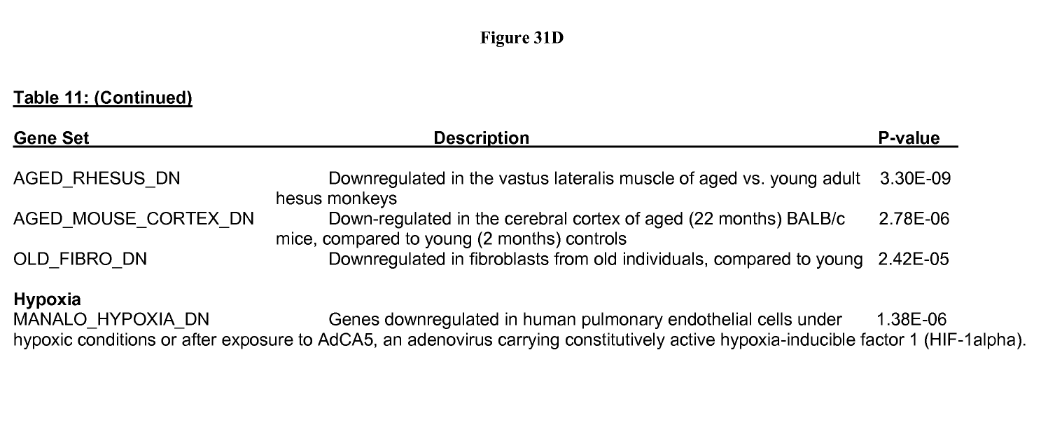

In one aspect, the present invention provides biomarkers which are, for example, as set forth in FIG. 29. In another aspect, the biomarker is selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof.

In one aspect, the present invention provides methods for determining the expression levels of individual and/or combinations of the differentially expressed biomarker sequences in a biological sample that are indicative of the presence, or stage of the disease, or the efficacy of therapy. The method comprises contacting said sample with a polynucleotide probe or a polypeptide ligand under conditions effective for said probe or ligand to hybridize specifically to a nucleic acid or a polypeptide in said sample, and detecting the presence or absence of biomarker. In one embodiment, methods are provided to determine the amounts and/or the differentially expressed levels at which the marker sequences of the present invention are expressed in samples. Such methods can comprise contacting said sample with a polynucleotide probe or a polypeptide ligand under conditions effective for said probe to hybridize specifically to the nucleic acids in said sample, and detecting the amounts or differentially expressed level of the marker sequences. In one preferred embodiment, said polynucleotide probe is a polynucleotide designed to identify one of the marker sequences in selected from the group consisting of ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof. See FIG. 29. In another preferred embodiment, said polypeptide ligand is an antibody.

In another aspect, the present invention provides polypeptides encoded by the marker sequences, biologically active portions thereof, and polypeptide fragments suitable for use as immunogens to raise antibodies directed against polypeptides of the marker sequences of the present invention.

In another aspect, the present invention provides ligands directed to polypeptides and fragments thereof of the marker sequences of the present invention. Preferably, said polypeptide ligands are antibodies. Antibodies of the invention include, but are not limited to, polyclonal, monoclonal, multispecific, human, humanized, or chimeric antibodies, single chain antibodies, Fab fragments, Fv fragments F(ab′) fragments, fragments produced by a Fab expression library, anti-idiotypic antibodies, or other epitope binding polypeptide. Preferably, an antibody, useful in the present invention for the detection of the individual marker sequences (and optionally at least one additional neoplastic disease-specific marker), is a human antibody or fragment thereof, including scFv, Fab, Fab′, F(ab′), Fd, single chain antibody, of Fv. Antibodies, useful in the invention may include a complete heavy or light chain constant region, or a portion thereof, or an absence thereof.

Another aspect of the present invention provides a method of assessing whether a subject is suffering from or at risk of developing neoplastic disease including colon neoplastic disease by detecting the differential expression of the marker sequences of the present invention. In one embodiment, the diagnostic method comprises determining whether a subject has an abnormal mRNA or cDNA and/or protein level of the marker sequences. The method comprises detecting the expression level of the individual and/or the combinations of the marker sequences in a biological sample obtained from a patient.

In some embodiments, the present invention provides methods for detection of expression of biomarkers in some embodiments, expression is measured directly (e.g., at the nucleic acid or protein level). In some embodiments, expression is detected in tissue samples (e.g., biopsy tissue). In other embodiments, expression is detected in bodily fluids (e.g., including but not limited to, plasma, serum, whole blood, mucus, and urine). The present invention further provides panels and kits for the detection of biomarkers. In preferred embodiments, the presence of a biomarker is used to provide a prognosis to a subject. For example, the detection of a biomarker in neoplastic disease tissues may be indicative of a neoplastic disease that is or is not likely to metastasize. In addition, the expression level of a biomarker may be indicative of a transformed cell, cancerous tissue or a neoplastic disease likely to metastasize.

The information provided can also be used to direct the course of treatment. For example, if a subject is found to possess or lack a biomarker that is likely to metastasize, therapies can be chosen to optimize the response to treatment (e.g., for subjects with a high probability of possessing a metastatic neoplastic disease more aggressive forms of treatment can be used).

Biomarkers may be measured as up or down-regulated using the methods of the present invention, and can be further characterized using microarray (e.g., nucleic acid or tissue microarray), immunohistochemistry, Northern blot analysis, siRNA or antisense RNA inhibition, mutation analysis, investigation of expression with clinical outcome, as well as other methods disclosed herein.

In some embodiments, the present invention provides a panel for the analysis of a plurality of biomarkers. The panel allows for the simultaneous analysis of multiple biomarkers correlating with carcinogenesis, metastasis and/or angiogenesis associated with neoplastic disease. For example, a panel may include biomarkers identified as correlating with cancerous tissue, metastatic cancer, localized neoplastic disease that is likely to metastasize, pre-cancerous tissue that is likely to become cancerous, pre-cancerous tissue that is not likely to become cancerous, and cancerous tissues or cells likely or not likely to respond to treatment. Depending on the subject, panels may be analyzed alone or in combination in order to provide the best possible diagnosis and prognosis. Markers for inclusion on a panel are selected by screening for their predictive value using any suitable method, including but not limited to, those described in the illustrative examples below.

In other embodiments, the present invention provides an expression profile map comprising expression profiles of s (e.g., of various stages or progeny) or prognoses (e.g., likelihood to respond to treatment or likelihood of future metastasis). Such maps can be used for comparison with patient samples. Any suitable method may be utilized, including but not limited to, by computer comparison of digitized data. The comparison data is used to provide diagnoses and/or prognoses to patients.

In some preferred embodiments, biomarkers (e.g., including but not limited to, those disclosed herein) are detected by measuring the levels of the biomarker in cells and tissue (e.g., cancer cells and tissues). For example, in some embodiments, a biomarker are monitored using antibodies (e.g., antibodies generated according to methods described below) or by detecting a biomarker protein. In some embodiments, detection is performed on cells or tissue after the cells or tissues are removed from the subject. In other embodiments, detection is performed by visualizing the biomarker in cells and tissues residing within the subject.

In some preferred embodiments, biomarkers of the invention are detected by measuring the expression of corresponding mRNA in a tissue sample (e.g., cancerous tissue).

In some embodiments, RNA is detected by Northern blot analysis. Northern blot analysis involves the separation of RNA and hybridization of a complementary labeled probe.

In still further embodiments, RNA (or corresponding cDNA) of the biomarkers of the invention is detected by hybridization to a oligonucleotide probe. A variety of hybridization assays using a variety of technologies for hybridization and detection are available. For example, in some embodiments, a TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA of the biomarkers of the invention. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

In other embodiments, gene expression of a biomarker of the invention is detected by measuring the expression of the corresponding protein or polypeptide. Protein expression may be detected by any suitable method. In some embodiments, proteins are detected by immunohistochemistry. In other embodiments, proteins are detected by their binding to an antibody raised against the protein. The generation of antibodies is described below.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to neoplastic disease markers is utilized.

In other embodiments, the immunoassay described in U.S. Pat. Nos. 5,599,677 and 5,672,480; each of which is herein incorporated by reference.

In yet other embodiments, the present invention provides kits for the detection and characterization of biomarkers of the invention. In some preferred embodiments, the kit contains s. In some embodiments, the kits contain antibodies specific for a biomarker of the invention, in addition to detection reagents and buffers. In other embodiments, the kits contain reagents specific for the detection of mRNA or cDNA (e.g., oligonucleotide probes or primers). In preferred embodiments, the kits contain all of the components necessary to perform a detection assay, including all controls, directions for performing assays, and any necessary software for analysis and presentation of results.

In some embodiments, in vivo imaging techniques are used to visualize the expression of a biomarker of the invention in an animal (e.g., a human or non-human mammal). For example, in some embodiments, a biomarker mRNA or protein is labeled using an labeled antibody specific for the biomarker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the biomarkers of the present invention are described herein.

The in vivo imaging methods of the present invention are useful in the diagnosis of cancers that express a biomarker of the invention of the present invention (e.g., cancerous cells or tissue). In vivo imaging is used to visualize the presence of a biomarker. Such techniques allow for diagnosis without the use of a biopsy. The in vivo imaging methods of the present invention are also useful for providing prognoses to neoplastic disease patients. For example. the presence of a biomarker indicative of an aggressive neoplastic disease likely to metastasize or likely to respond to a certain treatment can be detected. The in vivo imaging methods of the present invention can further be used to detect a (e.g., one that has metastasized) in other parts of the body.

In some embodiments, reagents (e.g., antibodies) specific for biomarkers of the present invention are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In some embodiments, flow-cytometry is utilized to monitor (e.g., detect) a marker (e.g., a biomarker of the present invention) (See, e.g., Example 1). The use of flow-cytometry to identify and/or isolate and/or purify cell populations is well known in the art (See, e.g., Givan, Methods Mol Biol 263, 1-32 (2004)).

In other embodiments, antibodies are radioactively labeled. The use of antibodies for in vivo diagnosis is well known in the art. Sumerdon et al., (Nucl. Med. Biol 17:247-254 (1990) have described an optimized antibody-chelator for the radioimmunoscintographic imaging of tumors using Indium-111 as the label. Griffin et al., (J Clin One 9:631-640 (1991)) have described the use of this agent in detecting tumors in patients suspected of having recurrent neoplastic disease. The use of similar agents with paramagnetic ions as labels for magnetic resonance imaging is known in the art (Lauffer, Magnetic Resonance in Medicine 22:339-342 (1991)). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

The present invention relates to methods using the biomarkers of the invention as an assayable biomarker in cell, fluid, and/or tissue samples obtained from individuals having neoplastic disease or suspected to have neoplastic disease, including e.g., various breast neoplasms, cancers and tumors, and also including primary disease samples. In this embodiment, the present invention provides substrate specific enzyme assays that are performed on samples, e.g. a specimen obtained from a breast biopsy or aspiration, to determine the enzyme level and activity of the biomarkers of the invention in the samples.

Therapeutic Compounds

The markers and marker sets of the present invention assess the likelihood of short or long term survival in neoplastic disease patients, e.g., patients having breast neoplastic disease. Using this prediction, neoplastic disease therapies can be evaluated to design a therapy regimen best suited for patients.

Known angiogenesis inhibitors that may used in methods of the invention include, but are not limited to, both direct and indirect angiogenesis inhibitors such as Angiostatin, bevacizumab (Avastin), Arresten, Canstatin, Combretastatin, Endostatin, NM-3, Thrombospondin, Tumstatin, 2-methoxyestradiol, and Vitaxin, ZD1839 (Iressa; getfitinib), ZD6474, OS1774 (tarceva), CI1033, PKI1666, IMC225 (Erbitux), PTK787, SU6668, SU11248, Herceptin, Marimastat, COL-3, Neovastat, 2-ME, SU6668, anti-VEGF antibody, Medi-522 (Vitaxin II), tumstatin, arrestin, recombinant EPO, troponin I, EMD121974, and IFN CELEBREX® (celecoxib), and THALOMID® (thalidomide), have also been recognized as angiogenesis inhibitors (Kerbel et al., Nature Reviews, Vol. 2, October 2002, pp. 727). A further example of an anti-angiogenic compound includes, but is not limited to PD 0332991 (see Fry, D. W. et al. Specific inhibition of cyclin-dependent kinase 4/6 by PD 0332991 and associated antitumor activity in human tumor xenografts. Mol Cancer Ther. 2004;3:1427-1438). Suitable antiangiogenic compositions include, but are not limited to Galardin (GM6001, Glycomed, Inc., Alameda, Calif.), endothelial response inhibitors (e.g., agents such as interferon alpha, TNP-470, and vascular endothelial growth factor inhibitors), agents that prompt the breakdown of the cellular matrix (e.g., Vitaxin (human LM-609 antibody, Ixsys Co., San Diego, Calif.; Metastat, CollaGenex, Newtown, Pa.; and Marimastat BB2516, British Biotech), and agents that act directly on vessel growth (e.g., CM-101, which is derived from exotoxin of Group A Streptococcus antigen and binds to new blood vessels inducing an intense host inflammatory response; and Thalidomide). Preferred anti-angiogenic inhibitors include, for example, bevacizumab, getfitinib thalidomide, tarceva, celecoxib, erbitux, arrestin, recombinant EPO, troponin I, herceptin. Dosages and routes of administration for these Food and Drug Administration (FDA) approved therapeutic compound are known to those of ordinary skill in the art as a matter of the public record.

Several kinds of steroids have also been noted to exert antiangiogenic activity. In particular, several reports have indicated that medroxyprogesterone acetate (MPA), a synthetic progesterone, potently inhibited neovascularization in the rabbit corneal assay (Oikawa (1988) Cancer Lett. 43: 85). A pro-drug of 5FU, 5′-deoxy-5-fluorouridine (5DFUR), might be also characterized as an antiangiogenic compound, because 5′DFUR is converted to 5-FU by the thymidine phosphorylase activity of PD-ECGF/TP. 5′DFUR might be selectively active for PD-ECGF/TP positive tumor cells with high angiogenesis potential. Recent clinical investigations in showed that 5DFUR is likely to be effective for PD-ECGF/TP-positive tumors. It was showed that a dramatic enhancement of antitumor effect of 5′DFUR appeared in PD-ECGF/TP transfected cells compared with untransfected wild-type cells (Haraguchi (1993) Cancer Res. 53: 5680 5682). In addition, combined 5DFUR+MPA compounds are also effective antiangiogenics (Yayoi (1994) Int J Oncol. 5: 27 32). The combination of the 5′DFUR+MPA might be categorized as a combination of two angiogenesis inhibitors with different spectrums, an endothelial growth factor inhibitor and a protease inhibitor. Furthermore, in in-vivo experiments using DMBA-induced rat mammary carcinomas, 5′DFUR exhibited a combination effect with AGM-1470 (Yamamoto (1995) Oncol Reports 2:793 796).

Another group of antiangiogenic compounds for use in this invention include polysaccharides capable of interfering with the function of heparin-binding growth factors that promote angiogenesis (e.g., pentosan polysulfate).

Other modulators of angiogenesis include platelet factor IV, and AGM 1470. Still others are derived from natural sources collagenase inhibitor, vitamin D3-analogues, fumigallin, herbimycin A, and isoflavones.

Therapeutic agents for use in the methods of the invention include, for example, a class of therapeutic agents known as proteosome inhibitors. As used herein, the term “proteasome inhibitor” refers to any substance which directly inhibits enzymatic activity of the 20S or 26S proteasome in vitro or in vivo. In some embodiments, the proteasome inhibitor is a peptidyl boronic acid. Examples of peptidyl boronic acid proteasome inhibitors suitable for use in the methods of the invention are disclosed in Adams et al., U.S. Pat. No. 5,780,454 (1998), U.S. Pat. No. 6,066,730 (2000), U.S. Pat. No. 6,083,903 (2000); U.S. Pat. No. 6,297,217 (2001), U.S. Pat. No. 6,465,433 (2002), U.S. Pat. No. 6,548,668 (2003), U.S. Pat. No. 6,617,317 (2003), and U.S. Pat. No. 6,747,150 (2004), each of which is hereby incorporated by reference in its entirety, including all compounds and formulae disclosed therein. Preferably, the peptidyl boronic acid proteasome inhibitor is selected from the group consisting of: N(4 morpholine)carbonyl-.beta.-(1-naphthyl)-L-alanine-L-leucine boronic acid; N(8 quinoline)sulfonyl-.beta.-(1-naphthyl)-L-alanine-L-alanine-L-leucine boronic acid; N(pyrazine)carbonyl-L-phenylalanine-L-leucine boronic acid, and N(4 morpholine)-carbonyl-[O-(2-pyridylmethyl)]L-tyrosine-L-leucine boronic acid. In a particular embodiment, the proteasome inhibitor is N (pyrazine)carbonyl-L-phenylalanine-L-leucine boronic acid (bortezomib; VELCADE®; formerly known as MLN341 or PS-341).

Additional peptidyl boronic acid proteasome inhibitors are disclosed in Siman et al., international patent publication WO 99/30707; Bernareggi et al., international patent publication WO 05/021558; Chatterjee et al., international patent publication WO 05/016859; Furet et al., U.S. patent publication 2004/0167337; Furet et al., international patent publication 02/096933; Attwood et al., U.S. Pat. No. 6,018,020 (2000); Magde et al., international patent publication WO 04/022070; and Purandare and Laing, international patent publication WO 04/064755.

Additionally, proteasome inhibitors include peptide aldehyde proteasome inhibitors, such as those disclosed in Stein et al., U.S. Pat. No. 5,693,617 (1997); Siman et al., international patent publication WO 91/13904; Iqbal et al., J. Med. Chem. 38:2276-2277 (1995); and linuma et al., international patent publication WO 05/105826, each of which is hereby incorporated by reference in its entirety.

Additionally, proteasome inhibitors include peptidyl epoxy ketone proteasome inhibitors, examples of which are disclosed in Crews et al., U.S. Pat. No. 6,831,099; Smyth et al., international patent publication WO 05/111008; Bennett et al., international patent publication WO 06/045066; Spaltenstein et al. Tetrahedron Lett. 37:1343 (1996); Meng, Proc. Natl. Acad. Sci. 96: 10403 (1999); and Meng, Cancer Res. 59: 2798 (1999), each of which is hereby incorporated by reference in its entirety.

Additionally, proteasome inhibitors include alpha-ketoamide proteasome inhibitors, examples of which are disclosed in Chatterjee and Mallamo, U.S. Pat. No. 6,310,057 (2001) and U.S. Pat. No. 6,096,778 (2000); and Wang et al., U.S. Pat. No. 6,075,150 (2000) and U.S. Pat. No. 6,781,000 (2004), each of which is hereby incorporated by reference in its entirety.

Additional proteasome inhibitors include peptidyl vinyl ester proteasome inhibitors, such as those disclosed in Marastoni et al., J. Med. Chem. 48:5038 (2005), and peptidyl vinyl sulfone and 2-keto-1,3,4-oxadiazole proteasome inhibitors, such as those disclosed in Rydzewski et al., J. Med. Chem. 49:2953 (2006); and Bogyo et al., Proc. Natl. Acad. Sci. 94:6629 (1997), each of which is hereby incorporated by reference in its entirety.

Additional proteasome inhibitors include azapeptoids and hydrazinopeptoids, such as those disclosed in Bouget et al., Bioorg. Med. Chem. 11:4881 (2003); Baudy-Floc'h et al., international patent publication WO 05/030707; and Bonnemains et al., international patent publication WO 03/018557, each of which is hereby incorporated by reference in its entirety.

Furthermore, proteasome inhibitors include peptide derivatives, such as those disclosed in Furet et al., U.S. patent publication 2003/0166572, and efrapeptin oligopeptides, such as those disclosed in Papathanassiu, international patent publication WO 05/115431, each of which is hereby incorporated by reference in its entirety.

Further, proteasome inhibitors include lactacystin and salinosporamide and analogs thereof, which have been disclosed in Fenteany et al., U.S. Pat. No. 5,756,764 (1998), U.S. Pat. No. 6,147,223 (2000), U.S. Pat. No. 6,335,358 (2002), and U.S. Pat. No. 6,645,999 (2003); Fenteany et al., Proc. Natl. Acad. Sci. USA (1994) 91:3358; Fenical et al., international patent publication WO 05/003137; Palladino et al., international patent publication WO 05/002572; Stadler et al., international patent publication WO 04/071382; Xiao and Patel, U.S. patent publication 2005/023162; and Corey, international patent publication WO 05/099687, each of which is hereby incorporated by reference in its entirety.

Further, proteasome inhibitors include naturally occurring compounds shown to have proteasome inhibition activity can be used in the present methods. For example, TMC-95A, a cyclic peptide, and gliotoxin, a fungal metabolite, have been identified as proteasome inhibitors. See, e.g., Koguchi, Antibiot. (Tokyo) 53:105 (2000); Kroll M, Chem. Biol. 6:689 (1999); and Nam S, J. Biol. Chem. 276: 13322 (2001), each of which is hereby incorporated by reference in its entirety. Additional proteasome inhibitors include polyphenol proteasome inhibitors, such as those disclosed in Nam et al., J. Biol. Chem. 276:13322 (2001); and Dou et al., U.S. patent publication 2004/0186167, each of which is hereby incorporated by reference in its entirety.

Preferred proteasome inhibitors include, for example, bortezomib. Dosages and routes of administration for Food and Drug Administration (FDA) approved therapeutic compounds are known to those of ordinary skill in the art as a matter of the public record.

Preferred angiogenesis inhibitors and other anti-neoplastic disease compounds, for use in the methods of the invention include, for example, 17-AAG, Apatinib, Ascomycin, Axitinib, Bexarotene, Bortezomib, Bosutinib, Bryostatin 1, Bryostatin 2, Canertinib, Carboplatin, Cediranib, Cisplatin, Cyclopamine, Dasatinib, 17-DMAG, Docetaxel, Doramapimod, Dovitinib, Erlotinib, Everolimus, Gefitinib, Geldanamycin, Gemcitabine, Imatinib, Imiquimod, Ingenol 3-Angelate, Ingenol 3-Angelate 20-Acetate, Irinotecan, Lapatinib, Lestaurtinib, Nedaplatin, Masitinib, Mubritinib, Nilotinib, NVP-BEZ235, OSU-03012, Oxaliplatin, Paclitaxel, Pazopanib, Picoplatin, Pimecrolimus, PKC412, Rapamycin, Satraplatin, Sorafenib, Sunitinib, Tandutinib, Tivozanib, Thalidomide, Temsirolimus, Tozasertib, Vandetanib, Vargatef, Vatalanib, Zotarolimus, ZSTK474, Bevacizumab (Avasti). Cetuximab, Herceptin, Rituximab, Trastuzumab.

Preferred protein kinase inhibitors for use in the methods of the invention include, for example, Apatinib, Axitinib, Bisindolylmaleimide I, Bisindolylmaleimide I, Bosutinib, Canertinib, Cediranib, Chelerythrine, CP690550, Dasatinib, Dovitinib. Erlotinib, Fasudil, Gefitinib, Genistein, Go 6976, H-89, HA-1077, Imatinib, K252a, K252c, Lapatinib, Di-p-Toluenesulfonate, Lestaurtinib, LY 294002, Masitinib, Mubritinib, Nilotinib, OSU-03012, Pazopanib, PD 98059, PKC412, Roscovitine, SB 202190, SB 203580, Sorafenib, SP600125, Staurosporine, Sunitinib, Tandutinib, Tivozanib, Tozasertib, Tyrphostin AG 490, Tyrphostin AG 1478, U0126, Vandetanib, Vargatef Vatalanib, Wortmannin, ZSTK474. Preferred Hedgehog and Smoothened (Smo) Inhibitors for use in the methods of the invention include, for example, Cyclopamine.

Platinum-based Anti-Cancer Compounds for use in the methods of the invention include, for example, Carboplatin, Cisplatin, Eptaplatin, Nedaplatin, Oxaliplatin, Picoplatin, Satraplatin. Proteasome Inhibitors for use in the methods of the invention include, for example, Bortezomib (Velcade). Anti-Diabetes Drugs for use in the methods of the invention include, for example, Metformin.

Fibrosis Inhibitors for use in the methods of the invention include, for example, Halofuginone. Metformin, N-acetyl-cysteine (NAC). NfkB Inhibitors for use in the methods of the invention include, for example, RTA 402 (Bardoxolone methyl), Auranofin, BMS-345541, IMD-0354, PS-1145, TPCA-1, Wedelolactone. HIF Inhibitors for use in the methods of the invention include, for example, Echinomycin. Glycolysis Inhibitors for use in the methods of the invention include, for example, 2-deoxy-D-glucose (2-DG), 2-bromo-D-glucose, 2-fluoro-D-glucose, and 2-iodo-D-glucose, dichloro-acetate (DCA), 3-chloro-pyruvate, 3-Bromo-pyruvate (3-BrPA), 3-Bromo-2-oxopropionate, Oxamate.

PI-3 Kinase, Akt, and mTOR inhibitors for use in the methods of the invention include, for example, LY 294002, NVP-BEZ235, Rapamycin, Wortmannin. Isoflavones for use in the methods of the invention include, for example, Quercetin, and Resveratrol. Anti-Oxidants for use in the methods of the invention include, for example, N-acetyl-cysteine (NAC), N-acetyl-cysteine amide (NACA).

Immunosuppressants for use in the methods of the invention include, for example, Ascomycin, CP690550, Cyclosporin A, Everolimus, Fingolimod, FK-506, Mycophenolic Acid, Pimecrolimus, Rapamycin, Temsirolimus, Zotarolimus, and AR-C117977, which inhibits monocarboxylate transporter 1 (MCT1). Cyclin dependent kinase inhibitors (CDK) inhibitors for use in the methods of the invention include, for example, Roscovitine, and PD 0332991 (CDK4/6 inhibitor). Lysosomal acidification inhibitors for use in the methods of the invention include, for example, Chloroquine. PARP Inhibitors for use in the methods of the invention include, for example, BSI-201, Olaparib, DR 2313, NU 1025.

Abraxane® is an albumin-bound paclitaxel nanoparticles formulation as an injectable suspension for the treatment of metastatic breast cancer. It contains albumin-bound paclitaxel for the treatment of metastatic breast cancer. Schaumburg, III: Abraxis Oncology, a Division of American Pharmaceutical Partners, Inc; January 2005). See O'Shaughnessy, J. A. et al. (2004). “Weekly Nanoparticle Albumin Paclitaxel (Abraxane) Results in Long-Term Disease Control in Patients With Taxane-Refractory Metastatic Breast Cancer,” Breast Cancer Research and Treatment, 27.sup.th Annual Charles A. Coltman San Antonio Breast Cancer Symposium, San Antonio, Tex., Dec. 8-11, 2004, 88(1):S65, Abstract No. 1070.

Compounds described herein can be administered to a human patient per se, or in pharmaceutical compositions mixed with suitable carriers or excipient(s). Techniques for formulation and administration of the compounds of the instant application may be found in “Remington's Pharmaceutical Sciences,” Mack Publishing Co., Easton, Pa., latest edition. Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Pharmaceutical compositions suitable for use in the present invention include compositions wherein the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

Antibodies

The invention provides antibodies to biomarker proteins, or fragments of biomarker proteins. e.g., ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2. ADMA, 3-hydroxybutyrate, and combinations thereof. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, Fab, Fab and F(ab)2 fragments, and an Fab expression library, in general, an antibody molecule obtained from humans relates to any of the classes IgG, IgM, IgA, IgE and IgD, which differ from one another by the nature of the heavy chain present in the molecule. Certain classes have subclasses as well, such as IgG1, IgG2, and others. Furthermore, in humans, the light chain may be a kappa chain or a lambda chain. Reference herein to antibodies includes a reference to all such classes, subclasses and types of human antibody species.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining biomarker protein expression as well as biomarker activity, in the context of a biological sample (e.g., blood, serum, cells, tissue) to thereby determine whether an individual is afflicted with a disease or disorder, or is at risk of developing a disorder, associated with aberrant biomarker expression or activity. The disorders include cell proliferative disorders such as neoplastic disease. The invention also provides for prognostic (or predictive) assays for determining whether an individual is at risk of developing, a disorder associated with biomarker protein expression or activity. Such assays may be used for prognostic or predictive purpose to thereby prophylactically treat an individual prior to the onset of a disorder characterized by or associated with protein, nucleic acid expression, or biological activity, wherein the biomarker is e.g., ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof.

Another aspect of the invention provides methods for determining biomarker protein expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g. drugs, compounds) on the expression or activity of biomarker in clinical trials.

Diagnostic Assays

An exemplary method for detecting the presence or absence of the biomarkers of the invention in a biological sample involves obtaining a biological sample from a test subject and contacting the biological sample with a compound or an agent capable of detecting biomarker protein such that the presence of biomarker is detected in the biological sample, wherein the biological sample includes, for example, cells, and/or physiological fluids.

An agent for detecting biomarker protein is an antibody capable of binding to biomarker protein, preferably an antibody with a detectable label. Antibodies can be polyclonal, or more preferably, monoclonal. An intact antibody, or a fragment thereof (e.g., Fab or F(ab′)2) can be used. The term “labeled”, faith regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect biomarker protein in a biological sample in vitro as well as in vivo. For example, in vitro techniques for detection of biomarker protein include enzyme linked immunosorbent as (ELISA), Western blot, immunoprecipitation, and immunofluorescence. Furthermore, in vitro techniques for detection of biomarker protein include introducing into a subject a labeled anti-biomarker antibody. For example the antibody can be labeled with a radioactive marker whose presence and location in a subject can be detected by standard imaging techniques.

In another embodiment, the methods further involve obtaining a control biological sample from a control subject, contacting the control sample with a compound or agent capable of detecting biomarker protein, for example, ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, and combinations thereof, is detected in the biological sample, and comparing the presence of biomarker protein, or lack thereof in cells, for example control cells, compared to the control sample with the presence of biomarker protein, in the test sample.

The invention also encompasses kits for detecting the presence of the biomarkers of the invention in a biological sample. For example, the kit can comprise: a labeled compound or agent capable of detecting, for example, ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate protein in a biological sample, for example cells; means for determining the amount of the biomarkers of the invention in the sample; and means for comparing the amount of the biomarkers of the invention in the sample with a standard. The compound or agent can be packaged in a suitable container. The kit can further comprise instructions for using the kit to detect the biomarkers of the invention protein in, for example, cells.

In yet other embodiments, the present invention provides kits for the detection, characterization, and/or treatment of neoplastic disease. In some embodiments, the kits contain antibodies specific for biomarkers (e.g., ACLY, HMGCS1, HMGCS2, HMGCL, HMGCLL1, BDH1, BDH2, BNIP3, BNIP3L, miR-31, miR-34c, ACAT1, ACAT2, OXCT1, OXCT2, ADMA, 3-hydroxybutyrate, or combinations thereof). In some embodiments, the kits further contain detection reagents and buffers. In other embodiments, the kits contain reagents specific for the detection of nucleic acids (e.g., DNA, RNA, mRNA or cDNA, oligonucleotide probes or primers). In preferred embodiments, the kits contain all of the components necessary and/or sufficient to perform a detection assay, including all controls, directions for performing assays, and any necessary software for analysis and presentation of results.

Prognostic Assays

The diagnostic methods described herein can furthermore be utilized to identify subjects having or at risk of developing a disease or disorder associated with aberrant expression or activity of the biomarkers of the invention. For example, the assays described herein. Such as the preceding diagnostic assays or the following assays, can be utilized to identify a subject having or at risk of developing a disorder associated with protein, nucleic acid expression or activity of the biomarkers of the invention. Alternatively, the prognostic assays can be utilized to identify a subject having or at risk for developing, a disease or disorder. Thus the invention provides method for identifying a disease or disorder in which a test sample is obtained from a subject and biomarker protein is detected, wherein the presence or absence of biomarker protein in cells is diagnostic for a subject having or at risk of developing a disease or disorder such as neoplastic disease or disorder. As used herein, a “test sample” refers to a biological sample obtained from a subject of interest. For example, a test sample can be a biological fluid (e.g. serum), cell sample, and/or tissue, including but not limited to cells.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder. For example, such methods can be used to determine whether a subject can be effectively treated with an agent for a disorder. Thus, the invention provides methods for determining whether a subject can be effectively treated with an agent for a disorder in which a test sample is obtained and biomarker protein expression or activity is detected (e.g., herein the presence of biomarker protein is diagnostic for a subject that can be administered the agent to treat, for example, a neoplastic disorder).

The methods described herein may be performed, for example, by utilizing pre-packaged diagnostic kits comprising at least one antibody reagent described herein, which may be conveniently used, e.g., in clinical settings to diagnose patients exhibiting symptoms or family history of a disease or illness.

The term “control” refers, for example, to a cell or group of cells that is exhibiting common characteristics for the particular cell type from which the cell or group of cells was isolated. A normal cell sample does not exhibit tumorigenic potential, metastatic potential, or aberrant growth in vivo or in vitro. A normal control cell sample can be isolated from tissues in a subject that is not suffering from neoplastic disease. It may not be necessary to isolate a normal control cell sample each time a cell sample is tested for neoplastic disease as long as the normal control cell sample allows for probing during the testing procedure. In some embodiments, the levels of expression of the protein markers in the cell sample are compared to the levels of expression of the protein markers in a normal control cell sample of the same tissue type as the cell sample.

A “control” refers, for example, to a sample of biological material representative of healthy, neoplastic disease-free animals, and/or cells or tissues. The level of the biomarkers of the invention in a control sample is desirably typical of the general population of normal, neoplastic disease-free animals or of a particular individual at a particular time (e.g. before, during or after a treatment regimen), or in a particular tissue. This sample can be removed from an animal expressly for use in the methods described in this invention, or can be any biological material representative of normal, neoplastic disease-free animals, including neoplastic disease-free biological material taken from an animal with neoplastic disease elsewhere in its body. A control sample can also refer to an established level of the biomarkers of the invention, representative of the neoplastic disease-free population, that has been previously established based on measurements from normal, neoplastic disease-free animals. In one embodiment, the control may be adjacent normal tissue. In one embodiment, the control may be any commonly used positive or negative controls. In one embodiment, the control is anon-invasive, non-metastatic control sample. Kits may also comprise, for example, positive and negative control samples for quality control purposes.

In preferred embodiments, the level of activity of one or more proteasomal peptidases in a test sample is used in conjunction with clinical factors other than proteasomal peptidase activity to diagnose a disease. In these embodiments, the level of proteasome activity measured in the test sample is compared to a reference value to determine if the levels of activity are elevated or reduced relative to the reference value. Preferably, the reference value is the proteasomal peptidase activity measured in a comparable sample from one or more healthy individuals. An increase or decrease in proteasome activity may be used in conjunction with clinical factors other than proteasomal peptidase activity to diagnose a disease.

The term “elevated levels” or “higher levels” as used herein refers to levels of a proteasome peptidase activity, that are higher than what would normally be observed in a comparable sample from control or normal subjects (i.e., a reference value). In some embodiments of the invention “control levels” (i.e. normal levels) refer to a range of biomarker or biomarker activity levels that would be normally be expected to be observed in a mammal that does not have a neoplastic disorder and “elevated levels” refer to biomarker or biomarker activity levels that are above the range of control levels. The ranges accepted as “elevated levels” or “control levels” are dependant on a number of factors. For example, one laboratory may routinely determine absolute levels of an activity of an enzyme in a sample that are different than the absolute levels obtained for the same sample by another laboratory. Also, different assay methods may achieve different value ranges. Value ranges may also differ in various sample types, for example different body fluids or by different treatments of the sample. One of ordinary skill in the art is capable of considering the relevant factors and establishing appropriate reference ranges for “control values” and “elevated values” of the present invention. For example, a series of samples from control subjects and subjects diagnosed with neoplastic disorders can be used to establish ranges that are “normal” or “control” levels and ranges that are “elevated” or “higher” than the control range.

Similarly, “reduced levels” or “lower levels” as used herein refer to levels of a biomarker or biomarker activity that are lower than what would normally be observed in a comparable sample from control or normal subjects (i.e., a reference value). In some embodiments of the invention “control levels” (i.e. normal levels) refer to a range of biomarker or biomarker activity levels that would be normally be expected to be observed in a mammal that does not have a hematological disorder and “reduced levels” refer to biomarker or biomarker activity levels that are below the range of such control levels.

Monitoring of Effects During Clinical Trials

Monitoring the influence of agents (e.g., drugs, compounds) on the expression or activity of the biomarkers of the invention (e.g., the ability to modulate aberrant cell proliferation and/or differentiation) can be applied not only in basic drug screening, but also in clinical trials. For example, the effectiveness of an agent determined by a screening assay as described herein to increase biomarker gene expression, protein levels, or upregulate biomarker activity, can be monitored in clinical trails of subjects exhibiting, for example, increased biomarker expression, protein levels, or downregulated biomarker activity or expression, for example in cells. Alternatively, the effectiveness of an agent determined by a screening assay to decrease biomarker expression, protein levels, or downregulate biomarker activity or expression, can be monitored in clinical trails of subjects exhibiting increased biomarker expression, protein levels, or upregulated biomarker activity. In such clinical trials, the expression or activity of biomarker and, preferably, other genes that have been implicated in, for example, a cellular proliferation or immune disorder can be used as a “read out” or markers of the immune responsiveness of a particular cell.

In one embodiment, the invention provides a method for monitoring the effectiveness of treatment of a subject with an agent (e.g., an agonist, antagonist, protein, peptide, peptidomimetic, nucleic acid, small molecule, or other drug candidate) comprising the steps of (i) obtaining a pre-administration sample from a subject prior to administration of the agent: (ii) detecting the level of expression of a biomarker protein, in the preadministration sample; (iii) obtaining one or more post-administration samples from the subject; (ill) detecting the level of expression or activity of the biomarker protein, in the post-administration samples; (v) comparing the level of expression or activity of the biomarker protein, in the pre-administration sample with the biomarker protein, in the post administration sample or samples; and (vi) altering the administration of the agent to the subject accordingly. For example, increased administration of the agent may be desirable to increase the expression or activity of biomarker to higher levels than detected, i.e., to increase the effectiveness of the agent. Alternatively, decreased administration of the agent may be desirable to decrease expression or activity of biomarker to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant expression or activity of the biomarkers of the invention. The disorders include, but are not limited to cell proliferative disorders such as neoplastic disease.

Prophylactic Methods

In one aspect, the invention provides a method for preventing, in a subject, a disease or condition associated with an aberrant expression or activity of the biomarkers of the invention, by administering to the subject an agent that modulates biomarkers expression or at least one biomarker activity, in for example cells. Subjects at risk for a disease that is caused or contributed to by aberrant biomarker expression or activity can be identified by, for example, any or a combination of diagnostic or prognostic assays as described herein. Administration of a prophylactic agent can occur prior to the manifestation of symptoms characteristic of the biomarker aberrancy, such that a disease or disorder is prevented or, alternatively, delayed in its progression. Depending upon the type of biomarker aberrancy, for example, a biomarker agonist or biomarker antagonist agent can be used for treating the subject. The appropriate agent can be determined based on screening assays described herein.

Therapeutic Methods

Another aspect of the invention pertains to methods of modulating expression or activity of the biomarkers of the invention in, for example cells, for therapeutic purposes. The modulatory method of the invention involves contacting a cell with an agent that modulates one or more of the activities of biomarker protein activity associated with the cell. An agent that modulates biomarker protein activity can be an agent as described herein, such as a nucleic acid or a protein, a naturally-occurring cognate ligand of a biomarker protein, a peptide. a biomarker peptidomimetic, or other small molecule. In one embodiment, the agent stimulates one or more biomarker protein activity. Examples of such stimulatory agents include active protein and a nucleic acid molecule encoding biomarker that has been introduced into the cell. In another embodiment, the agent inhibits one or more biomarker protein activity. Examples of such inhibitory agents include antisense biomarker nucleic acid molecules and anti-biomarker antibodies. These modulatory methods can be performed in vitro (e.g. by culturing the cell with the agent) or, alternatively, in vivo (e.g., by administering the agent to a subject). As such, the invention provides methods of treating an individual afflicted with a disease or disorder characterized by aberrant expression or activity of a biomarker protein molecule. In one embodiment, the method involves administering an agent (e.g., an agent identified by a screening assays described herein), or combination of agents that modulates (e.g., up-regulates or down-regulates) biomarker expression or activity.

Stimulation of biomarker activity is desirable in situations in which biomarker is abnormally downregulated and/or in which increased biomarker activity is likely to have a beneficial effect. One example of such a situation is where a subject has a disorder characterized by aberrant cell proliferation and/or differentiation (e.g., neoplastic disease or immune associated disorders).

Determination of the Biological Effect of the Therapeutic

In various embodiments of the invention, suitable in vitro or in vivo assays are performed to determine the effect of a specific therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Kits

As used herein, the term “label” encompasses chemical or biological molecules that are used in detecting the presence in a sample of a target molecule which is capable of binding to or otherwise interact with the label so as to indicate its presence in the sample, and the amount of the target molecule in the sample. Examples of such labels include, but not limited to, a nucleic acid probe such as a DNA probe. or RNA probe, an antibody, a radioisotope, a fluorescent dye, and the like.

As used herein, the term “usage instruction” includes instructions in the kit for carrying out the procedure for detecting the presence of a target molecular such as the biomarkers of the invention in the sample to be tested. In the context of kit being used in the United States, the usage instruction comprising the statement of intended use required by the U.S. Food and Drug Administration (FDA) in labeling in vitro diagnostic products. It would be apparent to one with ordinary skill in the art of medical diagnostic devices as to the format and content of these usage instructions as required by the FDA.

As used in the present invention, an appropriate binding assay for selecting specific biomarker-related angiogenesis inhibitor includes HPLC, immunoprecipitation, fluorescent-binding assay, capillary electrophoresis, and so forth.

As used herein, an “anti-angiogenesis assay” is an experiment where a pool of candidate molecules are screened in order to discover the effectiveness of the candidate molecules in inhibiting angiogenesis. In order to discover whether a molecule has anti-angiogenesis property, various methods can be applied to carry out the present invention. For example, proteins and peptides derived from these and other sources, including manual or automated protein synthesis, may be quickly and easily tested for endothelial proliferation inhibiting activity using a biological activity assay such as the bovine capillary endothelial cell proliferation assay. Other bioassays for inhibiting activity include the chick embryonic chorioallantoic membrane (CAM) assay, the mouse corneal assay, and the effect of administering isolated or synthesized proteins on implanted tumors. The chick CAM assay is described by O'Reilly, et al. in “Angiogenic Regulation of Metastatic Growth”, Cell, vol. 79(2), Oct. 21, 1994, pp. 315-328, which is hereby incorporated by reference in its entirety. Additional anti-angiogenesis assays for screening for angiogenesis inhibitors can be found in Yu, et al., PNAS, Vol. 101, No. 21, pp 8005-8010 (2004), which is hereby incorporated by reference in its entirety.

In some embodiments of the invention, methods such as flow cytometry as well as Enzyme-linked Immunosorbent Assay (ELISA) techniques are used for quantification of the biomarkers of the invention.

Detection of the protein molecule of the biomarkers of the invention can be performed using techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

For example, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In certain cases, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to neoplastic disease markers is utilized.

Antibodies specific for the biomarkers of the invention are made according to techniques and protocols well known in the art. The antibodies may be either polyclonal or monoclonal. The antibodies are utilized in well-known immunoassay formats, such as competitive and non-competitive immunoassays, including ELISA, sandwich immunoassays and radioimmunoassays (RIAs), to determine the presence or absence of the endothelial proliferation inhibitors of the present invention in body fluids. Examples of body fluids include but are not limited to blood, serum, peritoneal fluid, pleural fluid, cerebrospinal fluid, uterine fluid, saliva, and mucus.

The present invention provides isolated antibodies that can be used in the diagnostic kits in the detection of the biomarkers of the invention. In preferred embodiments, the present invention provides monoclonal antibodies that specifically bind to the biomarkers of the invention.

An antibody against the biomarkers of the invention in the present invention may be any monoclonal or polyclonal antibody, as long as it can recognize the protein. Antibodies can be produced by using the biomarkers of the invention or its analogues as the antigen using conventional antibody or antiserum preparation processes.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter with the antiserum and then measuring the activity of the labeling agent bound to the antibody. The cell fusion can be carried out according to known methods, for example, the method described by Koehler and Milstein (Nature 256:495 [1975]). As a fusion promoter, for example, Sendai virus (HVJ) or, preferably, polyethylene glycol (PEG), is used.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

Methods of linking an antibody to a second agent such as a cytotoxic agent in order to form a combination antibody, also know as an immunotoxic, is well known in the art. Two major advances in the immunotoxin field have been the use of the recombinant DNA technique to produce recombinant toxins with better clinical properties and the production of single-chain immunotoxins by fusing the DNA elements encoding combining regions of antibodies, growth factors, or cytokines to a toxin gene.

First-generation immunotoxins were constructed by coupling toxins to MAb or antibody fragments using a heterobifunctional cross-linking agent. It was also discovered that genetic engineering could be used to replace the cell-binding domains of bacterial toxins with the Fv portions of antibodies or with growth factors.

The present invention provides kits for the detection and characterization of the biomarkers of the invention in neoplastic disease diagnostics. In some embodiments, the kits contain antibodies specific for the biomarkers of the invention, in addition to detection reagents and buffers. In other embodiments, the kits contain reagents specific for the detection of the biomarkers of the invention. In preferred embodiments, the kits contain all of the components necessary to perform a detection assay, including all controls, directions for performing assays, and any necessary software for analysis and presentation of results.

Kits containing labels such as antibodies against the biomarkers of the invention for measurement of the biomarkers of the invention are also contemplated as part of the present invention. Antibody solution is prepared such that it can detect the presence of biomarkers peptides in extracts of plasma, urine, tissues, and in cell culture media are further examined to establish easy to use kits for rapid, reliable, sensitive, and specific measurement and localization of the biomarkers of the invention. These assay kits include but are not limited to the following techniques; competitive and non-competitive assays, radioimmunoassay, bioluminescence and chemiluminescence assays, fluorometric assays, sandwich assays, immunoradiometric assays, dot blots, enzyme linked assays including ELISA, microtiter plates, antibody coated strips or dipsticks for rapid monitoring of urine or blood, and immunocytochemistry. For each kit the range, sensitivity, precision, reliability, specificity and reproducibility of the assay are established according to industry practices that are commonly known to and used by one with ordinary skill in the art.

This immunohistochemistry kit provides instructions, biomarker molecules, preferably labeled and linked to a fluorescent molecule such as fluorescein isothiocyanate, or to some other reagent used to visualize the primary antiserum. Immunohistochemistry techniques are well known to those skilled in the art. This immunohistochemistry kit permits localization of the biomarkers of the invention in tissue sections and cultured cells using both light and electron microscopy. It is used for both research and clinical purposes. For example, tumors are biopsied or collected and tissue sections cut with a microtome to examine sites of biomarker production. Such information is useful for diagnostic and possibly therapeutic purposes in the detection and treatment of neoplastic disease.

Diagnostic Applications

The subject compositions may be used in a variety of diagnostic applications. Exemplary embodiments of such diagnostic applications are described below.

As noted above, the present invention is based on the discovery that expression of the biomarkers of the invention in cells and/or fluids is increased in cells of high metastatic potential relative to cells of low metastatic potential, cells of non-metastatic potential, and to normal cells. In general, the terms “high metastatic potential” and “low metastatic potential” are used to describe the relative ability of a cell to give rise to metastases in an animal model, with “high metastatic potential” cells giving rise to a larger number of metastases and/or larger metastases than “low metastatic potential” cells. Thus, a cell of high metastatic potential poses a greater risk of metastases to the subject than a cell of low metastatic potential. “Non-metastatic cells” are those cells that are cancerous, but that do not develop detectable metastases following injection in an animal model.

The invention thus features methods and compositions for diagnosis and prognosis, as well as grading and staging of cancers, by detection of expression or activity of the biomarkers of the invention in a biological test sample, e.g, cell sample or tissue sample. The methods of the invention can also be used to monitor patients having a predisposition to develop a particular neoplastic disease, e.g., through inheritance of an allele associated with susceptibility to a neoplastic disease (e.g., BRCA1, BRCA2, TP53, ATM, or APC for breast cancer). Detection and monitoring of expression or activity levels the biomarkers of the invention can be used to detect potentially malignant events at a molecular level before they are detectable at a gross morphological level.

In general, diagnosis, prognosis, and grading and/or staging of cancers may be performed by a number of methods to determine the relative level of expression of the differentially expressed biomarker gene at the transcriptional level, and/or the absence or presence or altered amounts of a normal or abnormal biomarker polypeptide in patient cells. As used herein, “differentially expressed gene” is intended to refer to a gene having an expression level (e.g., which in turn is associated with a level of biomarker polypeptide production and/or biomarker transcription) that is associated with a decrease in expression level of at least about 25%, usually at least about 50% to 75%, more usually at least about 90% or more. In general, such a decrease in differentially expressed biomarker is indicative of the onset or development of the metastatic phenotype

“Diagnosis” as used herein generally includes determination of a subject's susceptibility to a disease or disorder, determination as to whether a subject is unaffected, susceptible to, or presently affected by a disease or disorder, and/or to identify a tumor as benign. non-cancerous, or cancerous (e.g. non-metastatic or metastatic, e.g., high metastatic potential or low metastatic potential). “Prognosis” is used herein to generally mean a determination of the severity of disease (e.g., identification or pre-metastatic or metastatic cancerous states, stages of cancer. etc.), which in turn can be correlated with the potential outcome, response to therapy, etc. A complete diagnosis thus can include diagnosis as discussed above, as well as determination of prognosis, cancer staging, and tumor grading. The present invention particularly encompasses diagnosis and prognosis of subjects in the context of cancers of various origins, particularly breast cancer (e.g., carcinoma in situ (e.g., ductal carcinoma in situ), estrogen receptor (ER)-positive breast cancer, ER-negative breast cancer, or other forms and/or stages of breast cancer) and prostate cancer.

“Sample” or “biological sample” as used throughout here are generally meant to refer to samples of biological fluids or tissues, particularly samples obtained from tissues, especially from cells of the type associated with the disease for which the diagnostic application is designed (e.g., cells, and/or ductal adenocarcinoma), and the like. “Samples” is also meant to encompass derivatives and fractions of such samples (e.g., cell lysates). Where the sample is solid tissue, the cells of the tissue can be dissociated or tissue sections can be analyzed.

Methods of the subject invention useful in diagnosis or prognosis typically involve comparison of the amount of gene product of the biomarkers of the invention in a sample of interest with that of a control to detect relative differences in the expression of the gene product, where the difference can be measured qualitatively. and/or quantitatively. Quantitation can be accomplished, for example, by comparing the level of expression product detected in the sample with the amounts of product present in a standard curve. A comparison can be made visually using ELISA to detect relative amounts of biomarker polypeptides in test and control samples; by using a technique such as densitometry, with or without computerized assistance, to detect relative amounts of detectably labeled biomarker polypeptides; or by using an array to detect relative levels of anti-biomarker polypeptide antibody binding, and comparing the pattern of antibody binding to that of a control.

In some embodiments of the methods of the invention it may be particularly desirable to detect expression of a biomarker gene product as well as at least one gene product.

Other gene products that can serve as controls or increase the sensitivity of classification of the metastatic phenotype of a cell, as well as gene products that can serve as controls for identification of normal cells (e.g., gene products that are expressed in normal cells but not in cancerous cells, or expressed in normal cells, but not in metastatic cells, etc.) are known in the art. In addition, the cells can be classified as normal or cancerous based on conventional methodologies such as general morphology as determined by light microscopy. For example, conventional techniques for classifying a cell as cancerous based on morphology can be performed prior to or simultaneously with detection of biomarker expression. Thus, a cell that exhibits abnormal morphology associated with the neoplastic disease phenotype, and that expresses a low level of biomarker relative to a normal cells or in which biomarker expression is not detectable is identified as a cell of high metastatic potential.

Methods for qualitative and quantitative detection of biomarker polypeptides in a sample, as well as methods for comparing such to control samples are well known in the art. The patient from whom the sample is obtained can be apparently healthy, susceptible to disease (e.g., as determined by family history or exposure to certain environmental factors), or can already be identified as having a condition in which altered expression of a gene product of the invention is implicated.

In the assays of the invention, the diagnosis can be determined based on detected gene product expression levels of the biomarkers of the invention, and may also include detection of additional diagnostic markers and/or reference sequences. Where the diagnostic method is designed to detect the presence or susceptibility of a patient to metastatic cancer, the assay preferably involves detection of a biomarker gene product and comparing the detected gene product levels to a level associated with a normal sample, to levels associated with a low metastatic potential sample, and/or to level associated with a high metastatic potential sample. For example, detection of a higher level of biomarker expression relative to a normal level is indicative of the presence in the sample of a cell having high metastatic potential. Given the disclosure provided herein, variations on the diagnostic and prognostic assays described herein will be readily apparent to the ordinarily skilled artisan.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely relation sequences (e. 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42.degree. C. in a solution consisting of 5×SSPE (43.8 g/1 NaCl, 6.9 g/1 NaH, PO₄.H₂O and 1.85 g/1EDTA, pH adjusted to 7.4 with NaOH), 0.5% SDS, 5.times. Denhardt's reagent and 100 microg/ml denatured salmon sperm DNA followed by washing in a solution comprising 0.1.times.SSPE, 1.0% SDS at 42° C. when a probe of about 500 nucleotides in length is employed.

“Medium stringency conditions” when used in reference to nucleic acid hybridization comprise conditions equivalent to binding or hybridization at 42.degree. C. in a solution consisting of 5.times.SSPE (43.8 g/1 NaCl, 6.9 g/1 NaH₂PO₄.H₂O and 1.85 g/1 EDTA, pH adjusted to 7.4 with NaOH), 0.5% SDS, 5.times. Denhardt's reagent and 100.mu.g/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42.degree. C. in a solution consisting of 5×SSPE (43.8 g/1 NaCl, 6.9 g/1 NaH₂PO₄H₂O and 1.85 g/1 EDTA, pH adjusted to 7.4 with NaOH), 0.1% SDS, 5.times. Denhardt's reagent (50.times. Denhardt's contains per 500 ml: 5 g Ficoll (Type 400, Pharamcia), 5 g BSA (Fraction V; Sigma)) and 100 microg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such that as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “probe” refers to an oligonucleotide (i.e., a sequence of nucleotides), whether occurring naturally as in a purified restriction digest or produced synthetically, recombinantly or by PC R amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

Any of a variety of detectable labels can be used in connection with the various methods of the invention. Suitable detectable levels include fluorochromes, radioactive labels, and the like. Suitable labels include, but are not necessarily limited to, fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. 32P, 35S, 3H; etc. The detectable label can involve a two stage system (e.g., biotin-avidin, hapten-anti-hapten antibody, etc.).

Reagents specific for the polynucleotides and polypeptides of the invention, such as detectably labeled antibodies or detectably labeled nucleotide probes, can be supplied in a kit for detecting the presence of an expression product in a biological sample. The kit can also contain buffers or labeling components, as well as instructions for using the reagents to detect and quantify expression products in the biological sample. Exemplary embodiments of the diagnostic methods of the invention are described below in more detail.

Polypeptide Detection in Diagnosis, Prognosis, Cancer Grading and Cancer Staging

In one embodiment, the test sample is assayed for the level of a polypeptide of the biomarkers of the invention. Diagnosis can be accomplished using any of a number of methods to determine the absence or presence or altered amounts of the differentially expressed polypeptide in the test sample. For example, detection can utilize staining of cells or histological sections (e.g., from a biopsy sample) with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.). The absence or presence of antibody binding can be determined by various methods, including flow cytometry of dissociated cells, microscopy, radiography, scintillation counting, etc. Any suitable alternative methods can of qualitative or quantitative detection of levels or amounts of differentially expressed polypeptide can be used, for example ELISA, western blot, immunoprecipitation, radioimmunoassay, etc.

In general, the detected level of biomarker polypeptide in the test sample is compared to a level of the differentially expressed gene product in a reference or control sample, e.g., in a normal cell or in a cell having a known disease state (e.g., cell of high metastatic potential).

Immunological Methods

In the context of the present invention, “immunological methods” are understood as meaning analytical methods based on immunochemistry, in particular on an antigen-antibody reaction. Examples of immunological methods include immunoassays such as radioimmunoassay (RIA), enzyme immunoassay (ER, combined with solid-phase technique: ELISA) or else immunofluorescence assays. The immunoassay is carried out by exposing the sample to be investigated to an SP-C-binding antibody and detecting and quantifying the amount of antibody which binds to SP-C. In these assays. detection and quantification is carried out directly or indirectly in a known manner. Thus, detection and quantification of the antigen-antibody complexes is made possible by using suitable labels which may be carried by the antibody directed against SP-C and/or by a secondary antibody directed against the primary antibody. Depending on the type of the abovementioned immunoassays, the labels are, for example, radioactive labels, fluorescent dyes or else enzymes. such as phosphatase or peroxidase, which can be detected and quantified with the aid of a suitable substrate.

In one embodiment of the invention, the immunological method is carried out with the aid of a suitable solid phase. Suitable solid phases which may be mentioned include the customary commercial microtiter plates made of polystyrene or membranes (for example made of polyvinylidene difluoride, PVDF) which are customarily used for the ELISA technique. Surprisingly, it has been found that even chromatography plates are suitable for use as solid phase in the process according to the invention. The implementation of the process according to the invention using chromatography plates is hereinbelow also referred to as immuno-TLC.

Screening for Targeted Drugs

In one embodiment, any of the biomarkers of the invention as described herein are used in drug screening assays. The biomarker proteins, antibodies, nucleic acids, modified proteins and cells containing the biomarkers of the invention are used in drug screening assays or by evaluating the effect of drug candidates on a “gene expression profile” or expression profile of polypeptides. In one embodiment, the expression profiles are used, preferably in conjunction with high throughput screening techniques to allow monitoring for expression profile genes after treatment with a candidate agent, Zlokamik, et al., Science 279, 84-8 (1998), Heid, et al., Genome Res., 6:986-994 (1996).

In another embodiment, the biomarker proteins, antibodies, nucleic acids, modified proteins and cells containing the native or modified biomarker proteins are used in screening assays. That is, the present invention provides novel methods for screening for compositions that modulate the cancer phenotype. This can be done by screening for modulators of gene expression or for modulators of protein activity. Similarly, this may be done on an individual gene or protein level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokamik, supra.

Having identified the biomarker genes herein, a variety of assays to evaluate the effects of agents on gene expression may be executed. In a preferred embodiment, assays may be run on an individual gene or protein level. That is, having identified a particular gene as aberrantly regulated in neoplastic disease, candidate bioactive agents may be screened to modulate the gene's regulation. “Modulation” thus includes both an increase and a decrease in gene expression or activity. The preferred amount of modulation will depend on the original change of the gene expression in normal versus tumor tissue, with changes of at least 10%. preferably 50%, more preferably 100-300%. and in some embodiments 300-1000% or greater. Thus, if a gene exhibits a 4 fold increase in tumor compared to normal tissue, a decrease of about four fold is desired; a 10 fold decrease in tumor compared to normal tissue gives a 10 fold increase in expression for a candidate agent is desired, etc. Alternatively, where the biomarkers of the invention has been altered but shows the same expression profile or an altered expression profile, the protein will be detected as outlined herein.

As will be appreciated by those in the art. this may be done by evaluation at either the gene or the protein level; that is, the amount of gene expression may be monitored using nucleic acid probes and the quantification of gene expression levels, or, alternatively, the level of the gene product itself can be monitored, for example through the use of antibodies to the biomarker protein and standard immunoassays. Alternatively, binding and bioactivity assays with the protein may be done as outlined below.

In a preferred embodiment, gene expression monitoring is done and a number of genes, i.e. an expression profile, is monitored simultaneously, although multiple protein expression monitoring can be done as well.

In this embodiment, the biomarker nucleic acid probes are attached to biochips as outlined herein for the detection and quantification of biomarker sequences in a particular cell. The assays are further described below.

Generally, in a preferred embodiment, a candidate bioactive agent is added to the cells prior to analysis. Moreover, screens are provided to identify a candidate bioactive agent that modulates a particular type of cancer, modulates biomarker proteins, binds to a biomarker protein, or interferes between the binding of a biomarker protein and an antibody.

The term “potential therapeutic agent” “candidate bioactive agent” or “drug candidate” or grammatical equivalents as used herein describes any molecule, e.g., protein, oligopeptide, small organic or inorganic molecule, polysaccharide, polynucleotide, etc., to be tested for bioactive agents that are capable of directly or indirectly altering either the cancer phenotype, binding to and/or modulating the bioactivity of a biomarker protein, or the expression of a biomarker sequence, including both nucleic acid sequences and protein sequences. In a particularly preferred embodiment, the candidate agent increases a biomarker phenotype, for example to a normal tissue fingerprint. Generally a plurality of assay mixtures are run in parallel with different agent concentrations to obtain a differential response to the various concentrations. Typically. one of these concentrations serves as a negative control, i.e., at zero concentration or below the level of detection.

In one aspect, a candidate agent will neutralize the effect of a biomarker protein. By “neutralize” is meant that activity of a protein is either inhibited or counter acted against so as to have substantially no effect on a cell.

Potential therapeutic agents encompass numerous chemical classes, though typically they are organic or inorganic molecules, preferably small organic compounds having a molecular weight of more than 100 and less than about 2,500 Daltons. Preferred small molecules are less than 2000, or less than 1500 or less than 1000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, or amidification to produce structural analogs.

In one embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and norleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of prokaryotic and eukaryotic proteins may be made for screening in the methods of the invention. Particularly preferred in this embodiment are libraries of bacterial, fungal, viral, and mammalian proteins, with the latter being preferred, and human proteins being especially preferred.

In another preferred embodiment, the candidate bioactive agents are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

In one embodiment, the candidate bioactive agents are nucleic acids. As described generally for proteins, nucleic acid candidate bioactive agents may be naturally occurring nucleic acids, random nucleic acids, or “biased” random nucleic acids. In another embodiment, the candidate bioactive agents are organic chemical moieties, a wide variety of which are available in the literature.

In assays for testing alteration of the expression profile of one or more biomarker genes, after the candidate agent has been added and the cells allowed to incubate for some period of time, a nucleic acid sample containing the target sequences to be analyzed is prepared. The target sequence is prepared using known techniques (e.g., converted from RNA to labeled cDNA, as described above) and added to a suitable microarray. For example, an in vitro reverse transcription with labels covalently attached to the nucleosides is performed. Generally, the nucleic acids are labeled with a label as defined herein, especially with biotin-FITC or PE, Cy3 and Cy5.

As will be appreciated by those in the art. these assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions that allow formation of the label probe hybridization complex only in the presence of target. Stringency can be controlled by altering a step parameter that is a thermodynamic variable, including, but not limited to, temperature, formamide concentration, salt concentration, chaotropic salt concentration, pH, organic solvent concentration, etc. These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents in the assays. These include reagents like salts, buffers, neutral proteins, e.g. albumin, detergents, etc which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target. In addition, either solid phase or solution based (i.e., kinetic PCR) assays may be used.

Once the assay is run, the data are analyzed to determine the expression levels, and changes in expression levels as between states, of individual genes, forming a gene expression profile.

In a preferred embodiment, as for the diagnosis and prognosis applications, having identified the differentially expressed gene(s) or mutated gene(s) important in any one state, screens can be run to test for alteration of the expression of the biomarker genes individually. That is, screening for modulation of regulation of expression of a single gene can be done. Thus, for example, in the case of target genes whose presence or absence is unique between two states, screening is done for modulators of the target gene expression.

In addition, screens can be done for novel genes that are induced in response to a candidate agent. After identifying a candidate agent based upon its ability to modulate a biomarker expression pattern leading to a normal expression pattern, or modulate a single biomarker gene expression profile so as to mimic the expression of the gene from normal tissue, a screen as described above can be performed to identify genes that are specifically modulated in response to the agent. Comparing expression profiles between normal tissue and agent treated tissue reveals genes that are not expressed in normal tissue, but are expressed in agent treated tissue. These agent specific sequences can be identified and used by any of the methods described herein for biomarker genes or proteins. In particular these sequences and the proteins they encode find use in marking or identifying agent-treated cells.

Thus, in one embodiment, a candidate agent is administered to a population of cells, that thus has an associated expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e. a peptide) may be put into a viral construct such as a retroviral construct and added to the cell, such that expression of the peptide agent is accomplished; see PCT US97/01019, hereby expressly incorporated by reference.

Once the candidate agent has been administered to the cells, the cells can be washed if desired and are allowed to incubate under preferably physiological conditions for some period of time. The cells are then harvested and a new gene expression profile is generated, as outlined herein.

In a preferred embodiment, screening is done to alter the biological function of the expression product of a biomarker gene. Again, having identified the importance of a gene in a particular state, screening for agents that bind and/or modulate the biological activity of the gene product can be run as is more fully outlined below.

In a preferred embodiment, screens are designed to first find candidate agents that can bind to biomarker proteins, and then these agents may be used in assays that evaluate the ability of the candidate agent to modulate the biomarker activity and the cancer phenotype. Thus, as will be appreciated by those in the art, there are a number of different assays that may be run; binding assays and activity assays.

In a preferred embodiment, binding assays are done. In general, purified or isolated gene product is used; that is, the gene products of one or more biomarker nucleic acids are made. In general, this is done as is known in the art. For example, antibodies are generated to the protein gene products, and standard immunoassays are run to determine the amount of protein present. Alternatively, cells comprising the biomarker proteins can be used in the assays.

Thus, in a preferred embodiment, the methods comprise combining a biomarker protein and a candidate bioactive agent, and determining the binding of the candidate agent to the biomarker protein. Preferred embodiments utilize the human or mouse biomarker protein, although other mammalian proteins may also be used, for example for the development of animal models of human disease. In some embodiments, as outlined herein, variant or derivative biomarker proteins may be used.

Generally, in a preferred embodiment of the methods herein, the biomarker protein or the candidate agent is non-diffusably bound to an insoluble support having isolated sample receiving areas (e.g. a microtiter plate, an array, etc.). The insoluble support may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon®, etc. Microtiter plates and arrays are especially convenient because a large number of assays can be carried out simultaneously, using small amounts of reagents and samples.

The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical cross linking. the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the biomarker protein is bound to the support, and a candidate bioactive agent is added to the assay. Alternatively, the candidate agent is bound to the support and the biomarker protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the candidate bioactive agent to the biomarker protein may be done in a number of ways. In a preferred embodiment, the candidate bioactive agent is labeled, and binding determined directly. For example, this may be done by attaching all or a portion of the biomarker protein to a solid support, adding a labeled candidate agent (for example a fluorescent label), washing off excess reagent, and determining whether the label is present on the solid support. Various blocking and washing steps may be utilized as is known in the art.

By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the proteins (or proteinaceous candidate agents) may be labeled at tyrosine positions using .sup.125I, or with fluorophores. Alternatively, more than one component may be labeled with different labels; using .sup.125I for the proteins, for example, and a fluorophore for the candidate agents.

In a preferred embodiment, the binding of the candidate bioactive agent is determined through the use of competitive binding assays. In this embodiment, the competitor is a binding moiety known to bind to the target molecule (i.e. the biomarkers of the invention), such as an antibody, peptide, binding partner, ligand, etc. Under certain circumstances, there may be competitive binding as between the bioactive agent and the binding moiety, with the binding moiety displacing the bioactive agent.

In one embodiment, the candidate bioactive agent is labeled. Either the candidate bioactive agent, or the competitor, or both, is added first to the protein for a time sufficient to allow binding, if present. Incubations may be performed at any temperature which facilitates optimal activity, typically between 4 and 40.degree. C. Incubation periods are selected for optimum activity, but may also be optimized to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the candidate bioactive agent. Displacement of the competitor is an indication that the candidate bioactive agent is binding to the biomarkers of the invention and thus is capable of binding to, and potentially modulating, the activity of the biomarkers of the invention. In this embodiment, either component can be labeled. Thus, for example, if the competitor is labeled, the presence of label in the wash solution indicates displacement by the agent. Alternatively, if the candidate bioactive agent is labeled, the presence of the label on the support indicates displacement.

In an alternative embodiment, the candidate bioactive agent is added first, with incubation and washing, followed by the competitor. The absence of binding by the competitor may indicate that the bioactive agent is bound to the biomarkers of the invention with a higher affinity. Thus, if the candidate bioactive agent is labeled, the presence of the label on the support, coupled with a lack of competitor binding, may indicate that the candidate agent is capable of binding to the biomarkers of the invention.

In a preferred embodiment, the methods comprise differential screening to identity bioactive agents that are capable of modulating the activity of the biomarkers of the invention. In this embodiment, the methods comprise combining a the biomarkers of the invention and a competitor in a first sample. A second sample comprises a candidate bioactive agent, a biomarkers of the invention and a competitor. The binding of the competitor is determined for both samples, and a change, or difference in binding between the two samples indicates the presence of an agent capable of binding to the biomarkers of the invention and potentially modulating its activity. That is, if the binding of the competitor is different in the second sample relative to the first sample, the agent is capable of binding to the biomarkers of the invention.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

Screening for agents that modulate the activity of the biomarkers of the invention may also be done. In a preferred embodiment, methods for screening for a bioactive agent capable of modulating the activity of the biomarkers of the invention comprise the steps of adding a candidate bioactive agent to a sample of the biomarkers of the invention, as above, and determining an alteration in the biological activity of proteins. “Modulating the activity of a the biomarkers of the invention” includes an increase in activity, a decrease in activity, or a change in the type or kind of activity present. Thus, in this embodiment, the candidate agent should both bind to the biomarkers of the invention (although this may not be necessary), and alter its biological or biochemical activity as defined herein. The methods include both in vitro screening methods, as are generally outlined above, and in vivo screening of cells for alterations in the presence, distribution, activity or amount of the biomarkers of the invention.

Thus, in this embodiment, the methods comprise combining a sample and a candidate bioactive agent, and evaluating the effect on activity of the biomarkers of the invention. By “biomarker activity” or grammatical equivalents herein is meant one of the biomarker protein's biological activities, including, but not limited to, its role in tumorigenesis, including cell division, preferably in lymphatic tissue, cell proliferation, tumor growth and transformation of cells.

In a preferred embodiment, the activity of the biomarkers of the invention is increased; in another preferred embodiment, the activity of the biomarkers of the invention is increased. Thus, bioactive agents that are antagonists are preferred in some embodiments, and bioactive agents that are agonists may be preferred in other embodiments.

In a preferred embodiment, the invention provides methods for screening for bioactive agents capable of modulating the activity of the biomarkers of the invention. The methods comprise adding a candidate bioactive agent, as defined above, to a cell comprising biomarker proteins. Preferred cell types include almost any cell. The cells contain a recombinant nucleic acid that encodes a biomarker protein. In a preferred embodiment, a library of candidate agents is tested on a plurality of cells.

In one aspect, the assays are evaluated in the presence or absence or previous or subsequent exposure of physiological signals, for example hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, bioactive agents are identified. Compounds with pharmacological activity are able to enhance or interfere with the activity of the biomarkers of the invention.

Animal Models and Transgenics

In another preferred embodiment the biomarkers of the invention find use in generating animal models of cancers. As is appreciated by one of ordinary skill in the art, gene therapy technology wherein antisense RNA directed to the biomarkers of the invention will diminish or repress expression of the gene. An animal generated as such serves as an animal model of biomarkers that finds use in screening bioactive drug candidates. Similarly, gene knockout technology, for example as a result of homologous recombination with an appropriate gene targeting vector, will result in the absence of the biomarker protein. When desired, tissue-specific expression or knockout of the biomarker protein may be necessary.

It is also possible that the biomarkers of the invention is overexpressed in neoplastic disease. As such, transgenic animals can be generated that overexpress the biomarkers of the invention. Depending on the desired expression level, promoters of various strengths can be employed to express the transgene. Also, the number of copies of the integrated transgene can be determined and compared for a deter ruination of the expression level of the transgene. Animals generated by such methods find use as animal models of the biomarkers of the invention and are additionally useful in screening for bioactive molecules to treat neoplastic disease.

The invention will be illustrated in more detail with reference to the following Examples, but it should be understood that the present invention is not deemed to be limited thereto.

Microarrays

Microarrays have become well known and extensively used in the art (See, e.g., Barinaga, Science 253: 1489 (1991); Bains, Bio/Technology 10: 757-758 (1992)). Guidance for the use of microarrays is provided by Wang, E et al., Nature Biotechnology 18; 457-459 (2000); Diehn M et al., Nature Genetics 25: 58-62 (2000).

Polynucleotides, polypeptides, or analogues are attached to a solid support or substrate, which may be made from glass, plastic (e.g., polypropylene, nylon), polyacrylamide, nitrocellulose, or other materials. “Substrate” refers to any suitable rigid or semi-rigid support to which polynucleotides or polypeptides are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores. Polynucleotides can be immobilized on a substrate by any method known in the art.

Among the vendors of microarrays and microarray technology usage are Affymetrix, Inc. (USA), NimbleGen Systems, Inc. (Madison, Wis., USA), and Incyte Genomics (USA); Agilent Technologies (USA) and Grafinity Pharmaceutical Design, GmbH (Germany); and CLONTECH Laboratories (Becton Dickinson Bioscience) and BioRobotics, Ltd. (Great Britain) (See, e.g., Gwynne and Heebner G, Science (2001)).

In some embodiments, microarrays are utilized to monitor the expression of genes from neoplastic disease (e.g., to compare expression to normal HSCs). In some embodiments, microarrays are used to monitor the progression of disease. Differences in gene expression between healthy (e.g., normal) HSCs and cancerous tissues can be identified or monitored by analyzing changes in patterns of gene expression compared with s (e.g., from a subject with neoplastic disease). In some embodiments, neoplastic disease can be diagnosed at earlier stages before the patient is symptomatic. The invention can also be used to monitor the efficacy of treatment. For example, when using a treatment with known side effects, a microarray can be employed to “fine tune” the treatment regimen. A dosage is established that causes a change in genetic expression patterns indicative of successful treatment. Expression patterns associated with undesirable side effects are avoided. This approach may be more sensitive and rapid than waiting for the patient to show inadequate improvement, or to manifest side effects, before altering the course of treatment.

Alternatively, animal models that mimic a disease, rather than patients, can be used to characterize expression profiles associated with a particular disease or condition. This gene expression data may be useful in diagnosing and monitoring the course of disease in a patient, in determining gene targets for intervention, and in testing novel treatment regimens.

Microarrays can be used to rapidly screen large numbers of candidate drug molecules, looking for ones that produce an expression profile similar to those of known therapeutic drugs, with the expectation that molecules with the same expression profile will likely have similar therapeutic effects. Thus, in some embodiments, the invention provides the means to determine the molecular mode of action of a drug.

U.S. Pat. Nos. 6,218,122, 6,165,709, and 6,146,830, each of which is herein incorporated by reference in their entireties, disclose methods for identifying targets of a drug in a cell by comparing (i) the effects of the drug on a wild-type cell, (ii) the effects on a wild-type cell of modifications to a putative target of the drug, and (iii) the effects of the drug on a wild-type cell which has had the putative target modified of the drug. In various embodiments, the effects on the cell can be determined by measuring gene expression, protein abundances, protein activities, or a combination of such measurements. In various embodiments, modifications to a putative target in the cell can be made by modifications to the genes encoding the target, modification to abundances of RNAs encoding the target, modifications to abundances of target proteins, or modifications to activities of the target proteins. The present invention provides an improvement to these methods of drug discovery by providing s, for a more precise drug discovery program.

An “expression profile” comprises measurement of a plurality of cellular constituents that indicate aspects of the biological state of a cell. Such measurements may include, e.g., RNA or protein abundances or activity levels. Aspects of the biological state of a cell of a subject, for example, the transcriptional state, the translational state, or the activity state, are measured. The collection of these measurements, optionally graphically represented, is called the “diagnostic profile”. Aspects of the biological state of a cell which are similar to those measured in the diagnostic profile (e.g., the transcriptional state) can be measured in an analogous subject or subjects in response to a known correlated disease state or, if therapeutic efficacy is being monitored, in response to a known, correlated effect of a therapy. The collection of these measurements, optionally graphically represented, is called herein the “response profile”. The response profiles are interpolated to predict response profiles for all levels of protein activity within the range of protein activity measured. In cases where therapeutic efficacy is to be monitored, the response profile may be correlated to a beneficial effect, an adverse effect, such as a toxic effect, or to both beneficial and adverse effects.

The invention will be illustrated in more detail with reference to the following Examples, but it should be understood that the present invention is not deemed to be limited thereto.

### EXAMPLES

### Example 1

### Metabolomic Analysis of Cav-1 (−/−) Null Tissues: Evidence for Oxidative Stress, Mitochondrial Dysfunction, and Autophagy

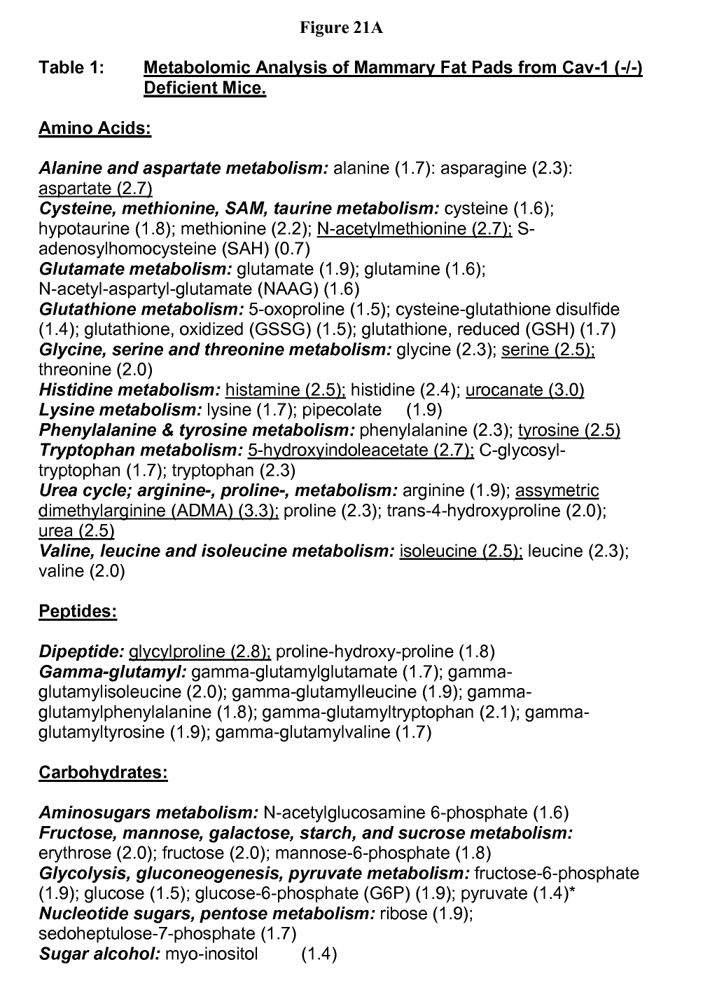

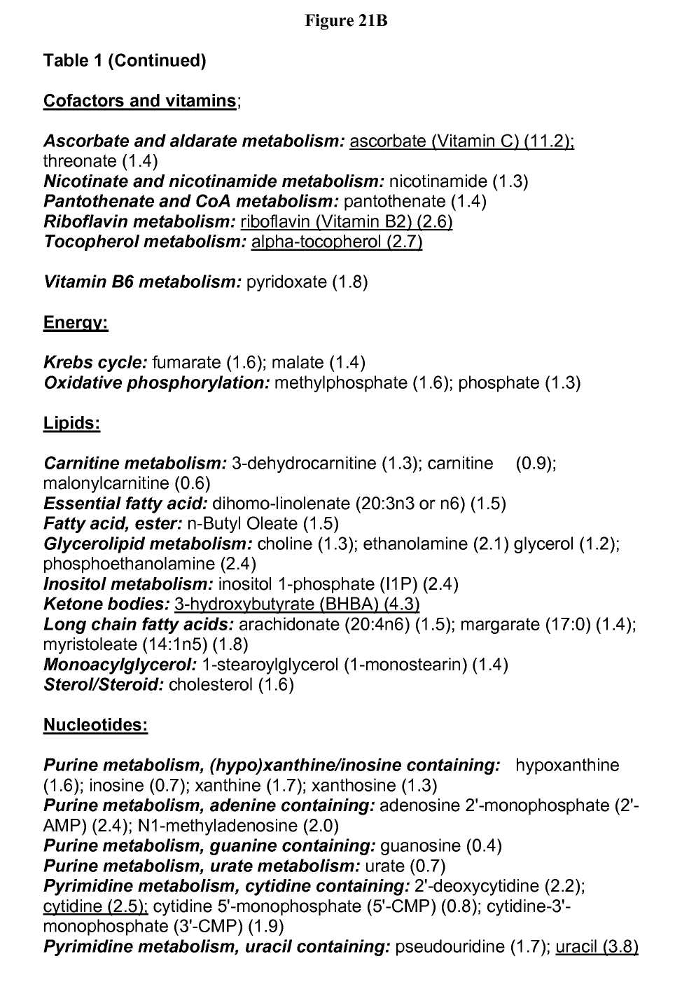

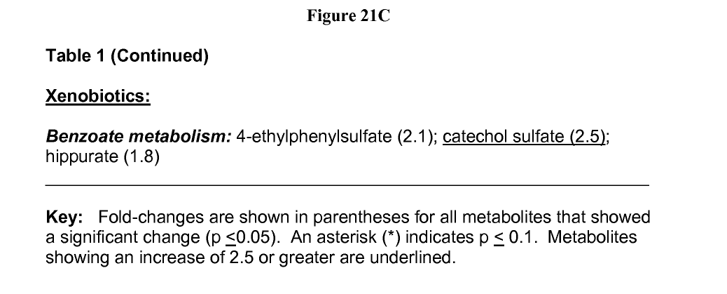

Mammary fat pads were harvested from age-matched female WT and Cav-1 (−/−) null mice (n=6 for each genotype) and subjected to an unbiased metabolomic analysis. Over 200 known compounds were identified by mass spectrometry analysis and were quantitated. Interestingly, there were a large number of compounds that were significantly changed in Cav-1 (−/−) mammary fat pads (n=103; 92 UP; 11 DOWN), consistent with a severe metabolic phenotype (Table 1, FIG. 21). Several observations are consistent with the presence of oxidative stress. These include: 1) an increase in the amounts of several anti-oxidants, such as ascorbic acid (˜11.2-fold), vitamin E (alpha-tocopherol; 2.7-fold), 5-hydroxyindoleacetate (2.7-fold), and hypotaurine (1.8-fold); 2) an increase in the number of amino-acid metabolites associated with the glutathione pathway, more specifically gamma-glutamyl amino acids and glutathione species (GSH, GSSH, 5-oxoproline, cys-glutathione-disulfide); 3) a shift towards gluconeogenesis, and the pentose phosphate pathway, which is known to produce increased amounts of NADPH, which can then be used as reducing equivalents to maintain reduced glutathione; 4) the observed increase in ribose and nucleotides, which emanate from the pentose phosphate pathway; and 5) an increase in the amount of ADMA (asymmetric dimethyl arginine; 3.3-fold), which is both a marker of protein catabolism and oxidative stress, and can also produce more oxidative stress. ADMA acts as an eNOS uncoupler, resulting in the production of superoxide, instead of nitric oxide. Similarly, ADMA is also a marker of chronic hypoxia and mitochondrial dysfunction.

As oxidative stress also drives mitochondrial dysfunction, autophagy, and mitophagy, evidence of these catabolic biological processes was examined in the metabolic data set. Consistent with a generalized catabolic phenotype, see: 1) higher levels of numerous amino acids and their catabolites; 2) elevation of 4 markers of protein or collagen degradation (assymetric dimethylarginine, trans-4-hydroxyproline, glycyl-proline, proline-hydroxy-proline); 3) elevated levels of a marker of increased RNA turnover, namely pseudouridine (1.7-fold); 4) increased levels (4.3-fold) of 3-hydroxybutryate (BHB), a ketone body, which is a well-accepted marker of mitochondrial dysfunction 22, 23; and 5) higher levels of free cholesterol (1.6-fold), which can also contribute to mitochondrial dysfunction 24. A decrease in mitochondrial function is also consistent with the accumulation of certain metabolites associated with glycolysis (pyruvate; 1.4-fold), and the TCA cycle (fumarate and malate; both >1.4-fold). Interestingly, increases in 5-hydroxyindole (2.7-fold). which is an anti-oxidant metabolite of tryptophan, was observed which protects against oxidative damage and mitochondrial dysfunction, as it suppresses ROS generation, lipid peroxidation, peroxynitrite generation, and glutathione depletion—thereby increasing mitochondrial membrane potential.

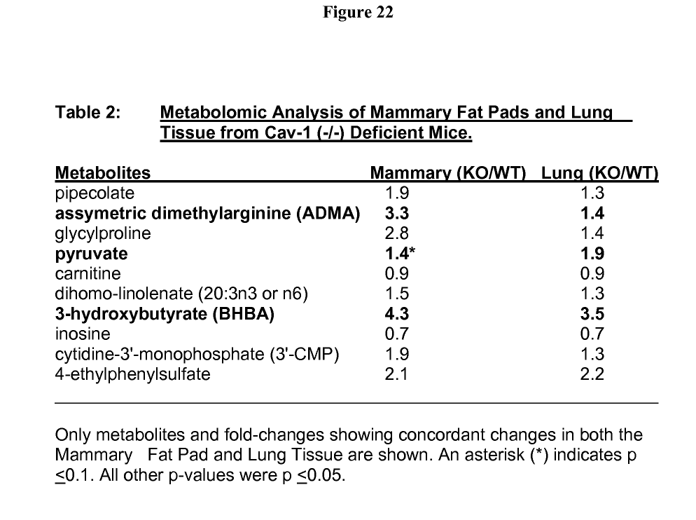

The results from the mammary fat pad were compared with lung tissue, as adipose tissue and lung tissue express the highest levels of Cav-1. Only concordant changes were selected and are shown in Table 2, FIG. 22. Interestingly, ADMA, pyruvate, and 3-hydroxybutyrate were significantly elevated in lung tissue, consistent with the idea that Cav-1 (−/−) null tissues are undergoing 1) oxidative stress and 2) mitochondrial dysfunction. Box plots for ADMA and BHB are shown in Figure ______, and for the antioxidant Vitamins C and E in Figure ______.

It is also known that oxidative stress is indeed sufficient to induce ketone production in an animal model of Amyotrophic Lateral Sclerosis (ALS). These mice express a mutant form of SOD1 (G86R) and show progressively increased serum levels of ketone bodies. Furthermore, ALS patients show increase serum levels of ketone bodies, both 3-hydroxybutyrate and acetone, as documented by NMR spectroscopy. Finally, autophagy has also been implicated in the pathogenesis of ALS, both using transgenic SOD1-mutant mouse models and human patient samples. Thus, oxidative stress, mitochondrial dysfunction, and autophagy/mitophagy are all clustered together in various neurodegenerative disorders, such as ALS and Alzheimer's disease.

Other noteworthy metabolites that were increased include histamine (2.5-fold) and arachidonic acid (1.5-fold), which may directly or indirectly contribute towards an inflammatory micro-environment. As arachidonic acid is the precursor of both prostaglandins and leukotrienes, increased free arachidonic acid could drive the generation of increased inflammatory mediators. Histamine also increases the differentiation of stromal cells towards a more myo-fibroblastic phenotype, consistent with the behavior of cancer-associated fibroblasts.

### Example 2

### Transcriptional mRNA Profiling of Cav-1 (−/−) Stromal Cells Provides Validating Evidence for a Stromal Catabolic State

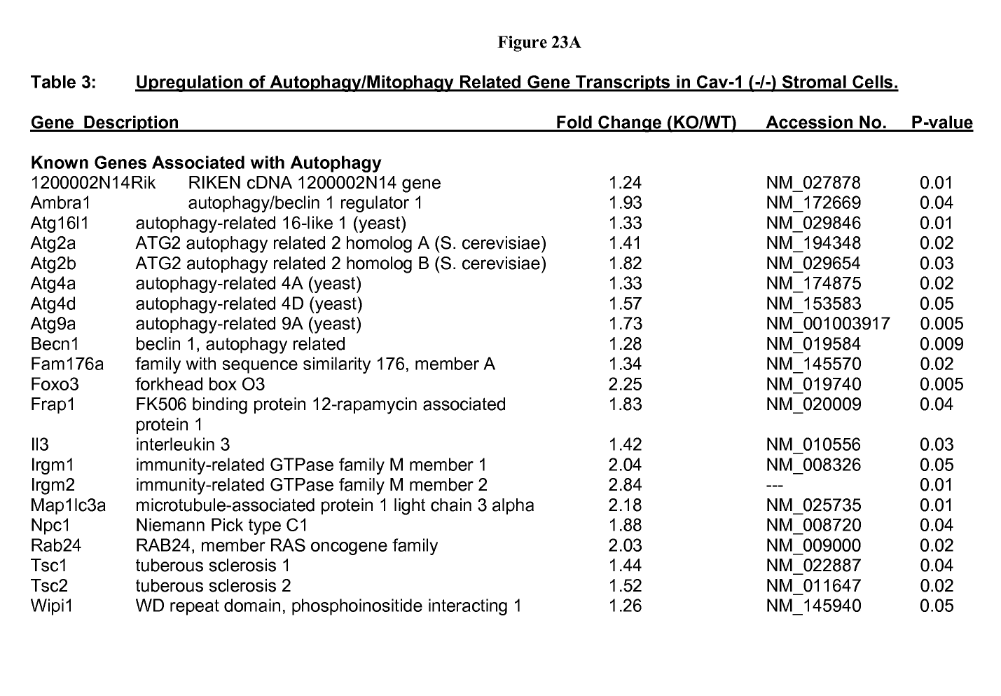

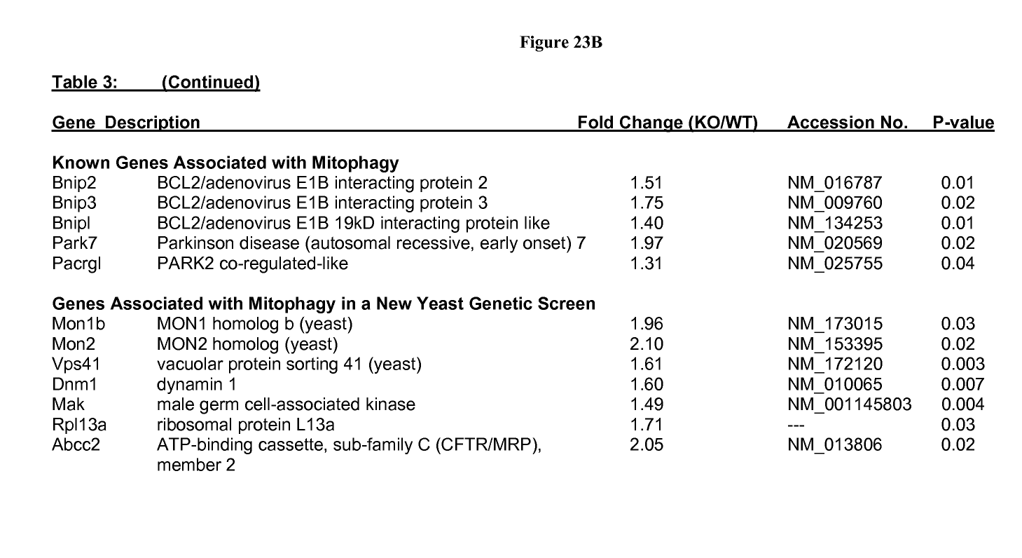

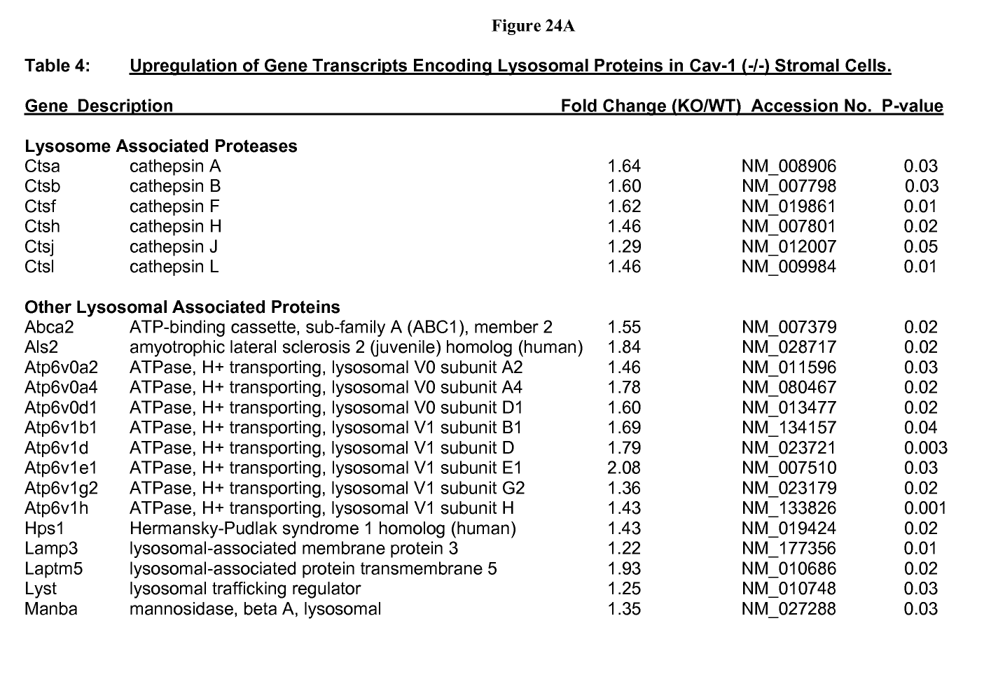

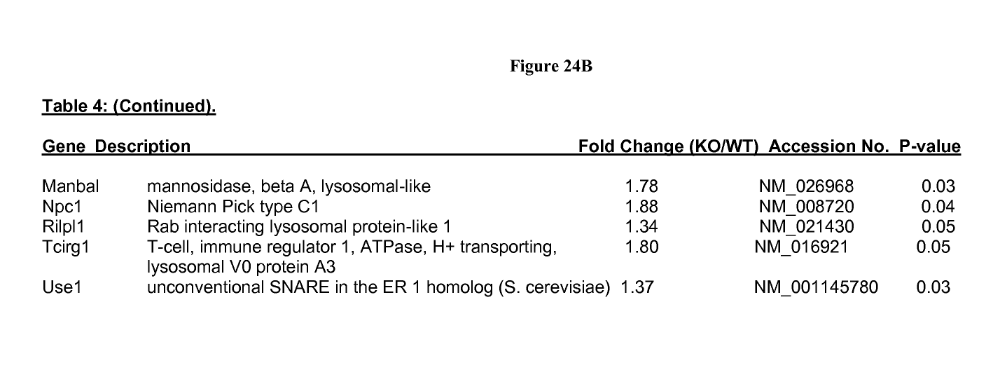

The inventors have previously traced the lethality of a Cav-1 negative tumor micro-environment to the stromal fibroblast or cancer-associated fibroblast compartment. Thus, to garner validating evidence for our metabolic profiling studies, the inventors re-interrogated the transcriptional profiling data obtained via the analysis of WT and Cav-1 (−/−) stromal cells. The metabolic features observed could be explained by oxidative stress induced autophagy and mitophagy. In direct support of this notion, Table 3, FIG. 23 shows that many the genes that are involved in mediating autophagy and mitophagy are indeed upregulated in Cav-1 (−/−) null stromal cells. Since autophagy and mitophagy are dependent on increased lysosomal degradation activity. the transcriptional profiles of lysosomal proteases (the cathepsins) and other lysosomal associated proteins was assessed (Table 4, FIG. 24). Interestingly, numerous cathepsin genes and lysomal associated proteins were transcriptionally upregulated in Cav-1 (−/−) stromal cells.

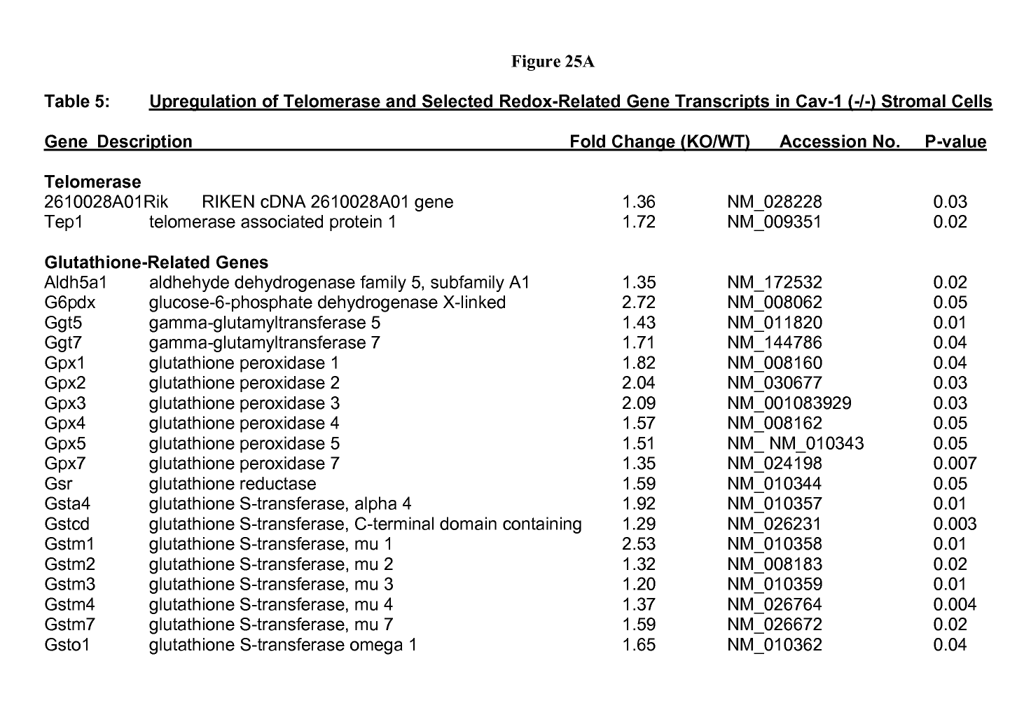

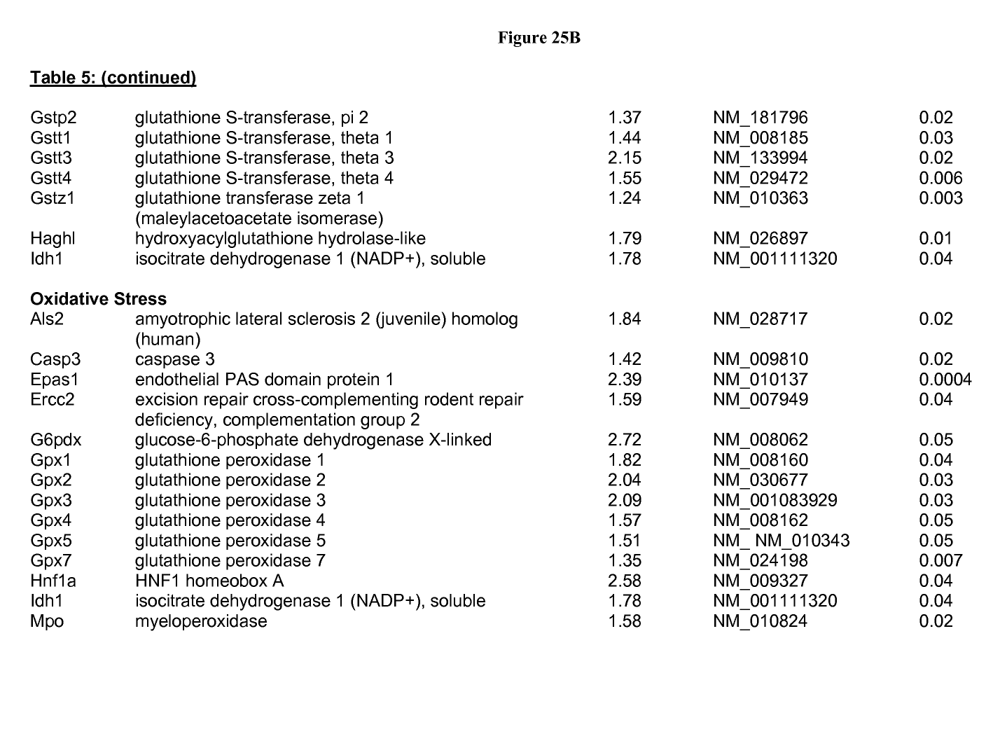

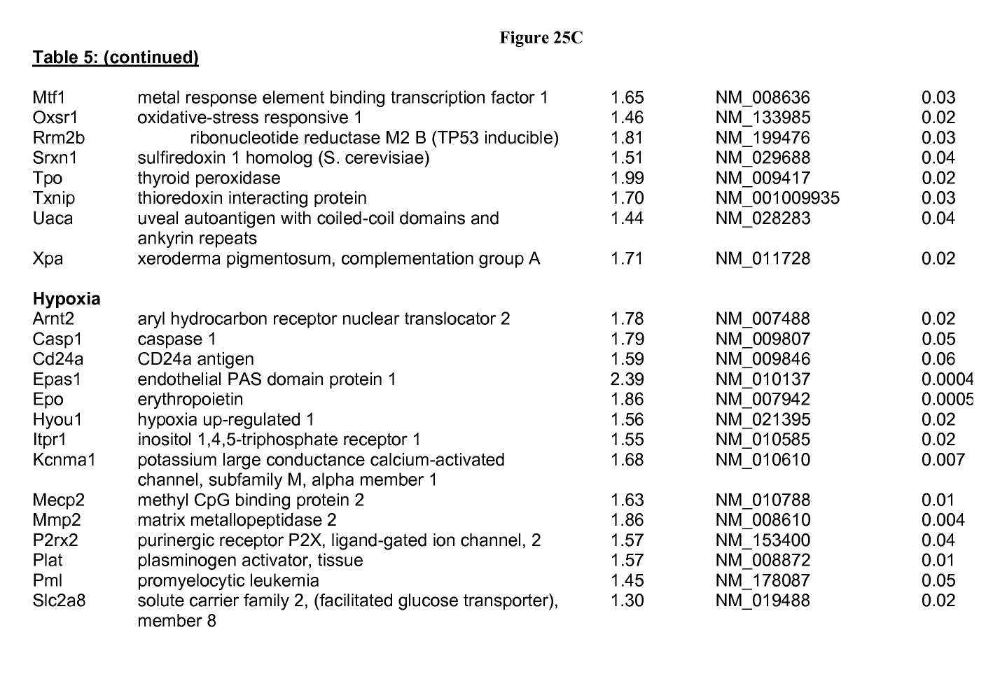

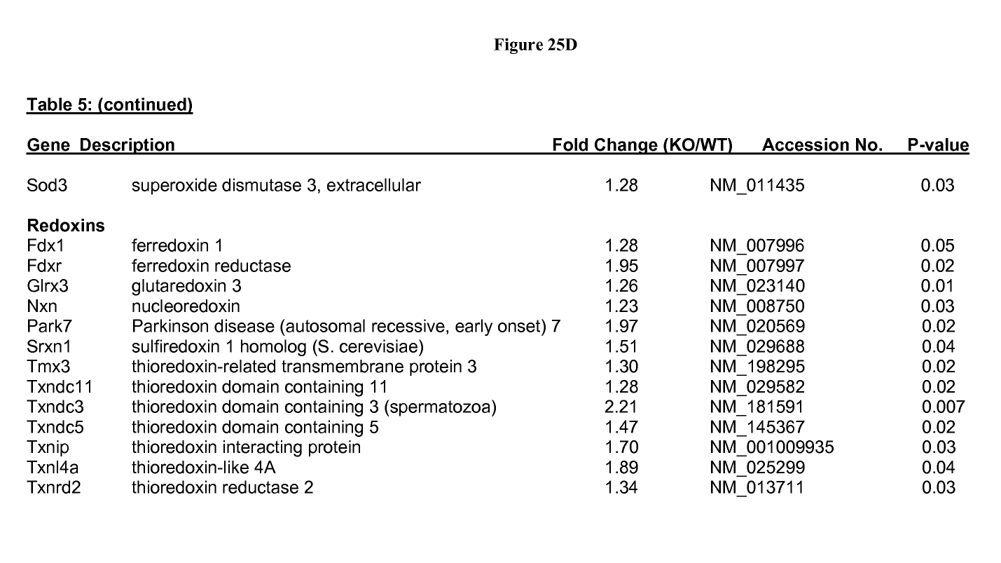

In further support of increased oxidative stress, the transcriptional overexpression of numerous genes associated with glutathione metabolism, genes responsive to oxidative stress) and hypoxia, as well as numerous anti-oxidant proteins (Table 5, FIG. 25). Thus, the transcriptional mRNA profile of Cav-1 (−/−) stromal cells is consistent with oxidative stress induced autophagy and mitophagy, and lysosome-related and peroxisome-related gene transcripts.

### Example 3

### Transcriptional mRNA Profiling of Human Breast Cancer Stroma Provides Evidence for Stromal Autophagy and Mitophagy In Vivo

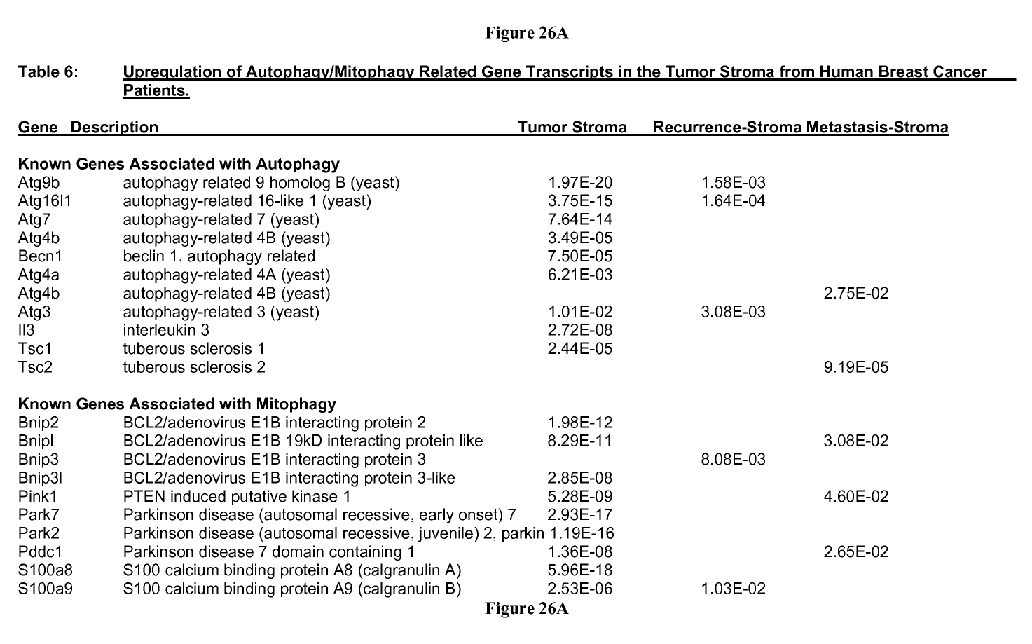

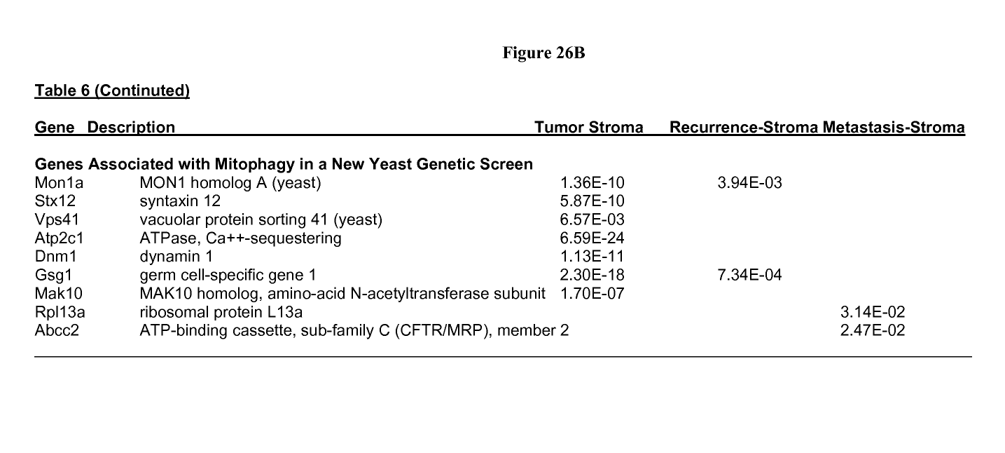

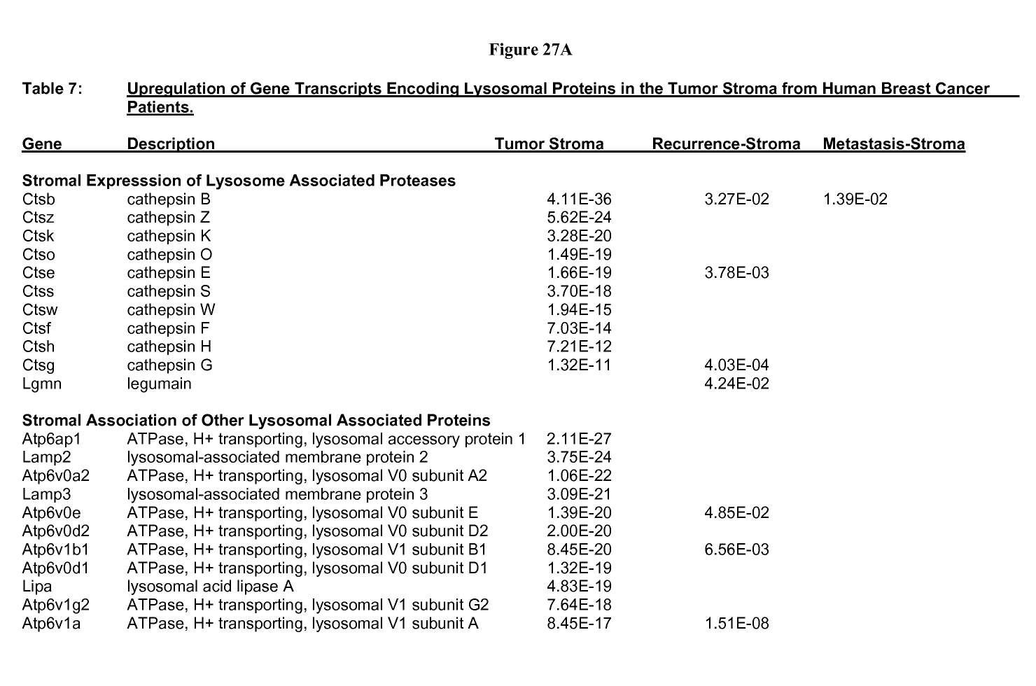

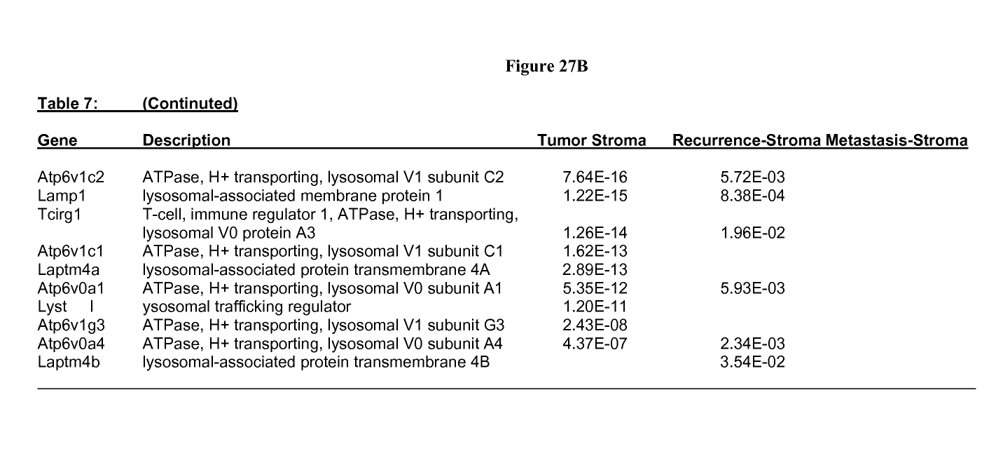

To further test the possible clinical relevance of our observations regarding autophagy and mitophagy, the transcriptional profiles of human tumor stroma that was isolated by laser-capture micro-dissection of breast cancer tumor tissue was analyzed. The methods and origins of these samples have been previously described in detail. Using these raw transcriptional profiling data, three related gene lists were created: 1) tumor stroma, 2) recurrence stroma, and 3) metastasis stroma. The tumor stroma list contains genes that were upregulated in the stroma of primary tumors, as compared with normal mammary gland stroma. The recurrence stroma list contains stromal genes, from the primary tumor, that are upregulated in patients that underwent tumor recurrence, as compared with patients that did not recur. The metastasis stromal list contains stromal genes, from the primary tumor, that are upregulated in patients that underwent lymph node metastasis at diagnosis, as compared with patients that did not show lymph node metastasis. Thus, these 3 complementary gene lists were analyzed for evidence of autophagy and mitophagy. Table 6 (FIG. 26) shows that many of the genes that are associated with autophagy and mitophagy are transcriptionally upregulated in the tumor stroma of human breast cancer patients. In further support of this idea, upregulation of lysosomal proteases (the cathepsins and) legumain), as well as other lysosomal associated proteins was observed (Table 7, FIG. 27).

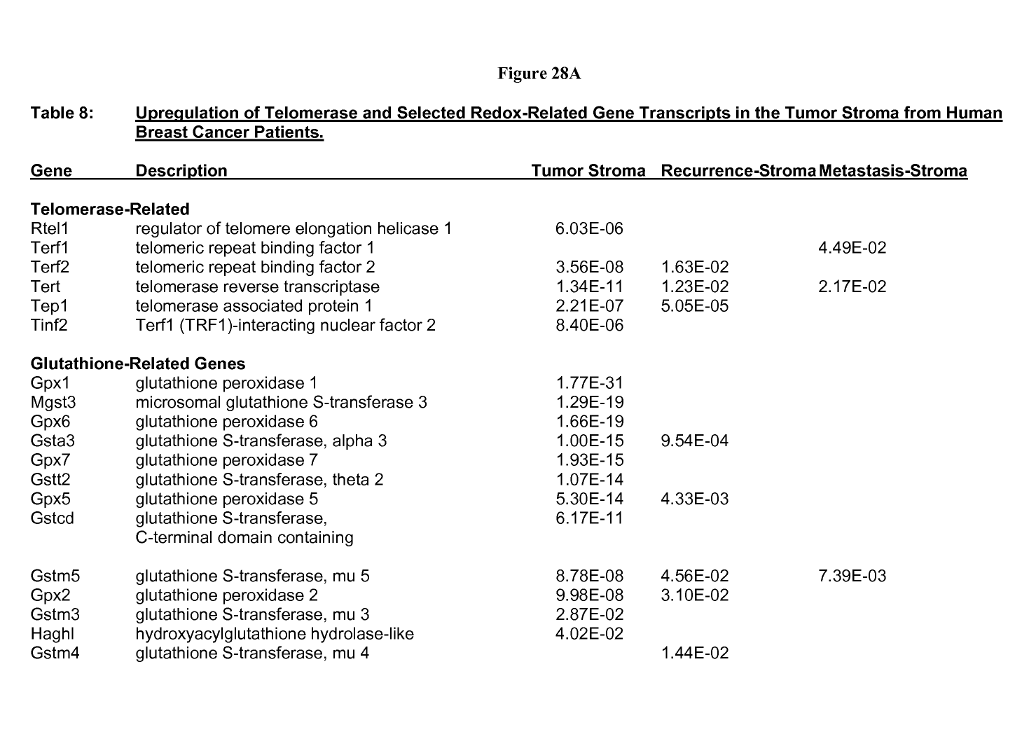

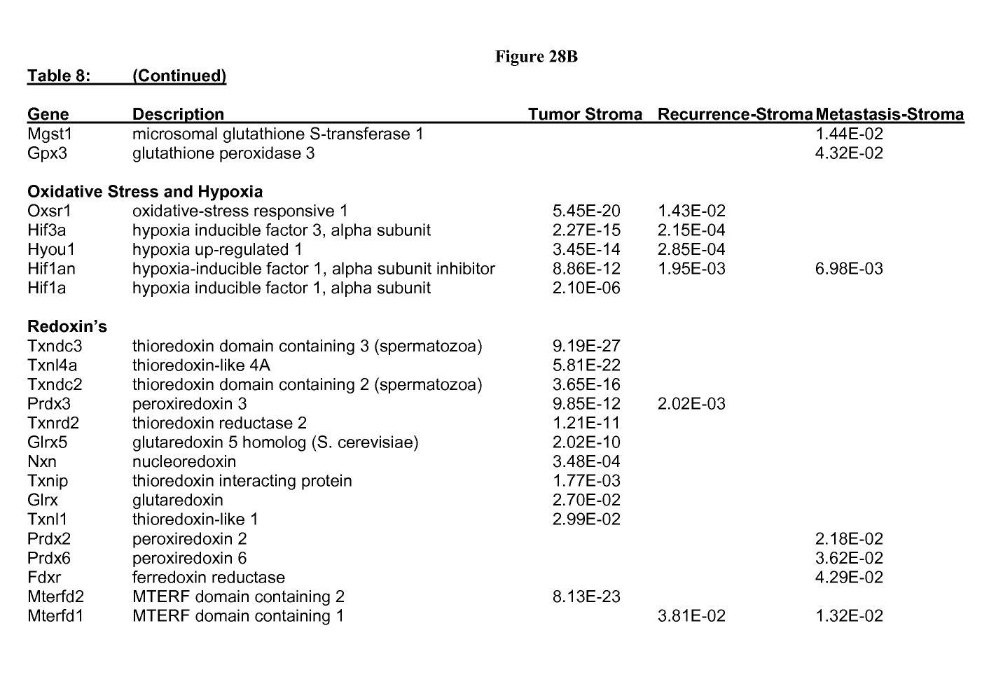

Finally, genes associated with glutathione metabolism, oxidative and hypoxic stress, as well as anti-oxidants are all transcriptionally upregulated in the tumor stroma obtained from human breast cancer patients (Table 8, FIG. 28). Perhaps. most interestingly, many of these gene transcripts are also associated with tumor recurrence and metastasis (See Tables 6-8, FIG. 28). It is important to note that telomerase-related genes are also transcriptionally upregulated in both Cav-1 (−/−) stromal cells and the tumor stroma of human breast cancers (See Tables 5 and 8, FIGS. 25 and 28). Thus, over-expression of telomerase activity could provide an escape mechanism to keep stromal cell cells alive for much longer periods of time under conditions of oxidative stress, autophagy, and mitophagy.

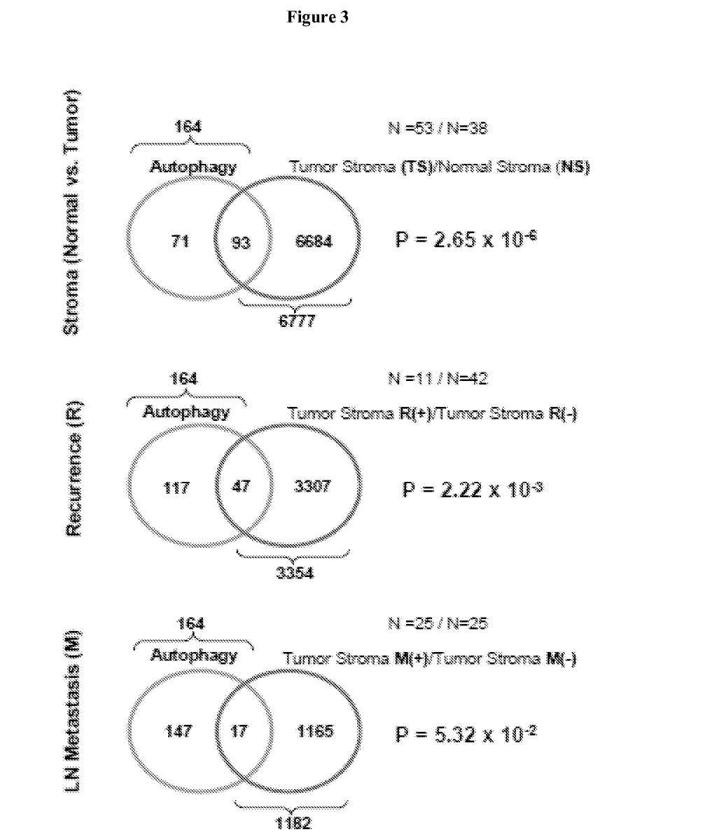

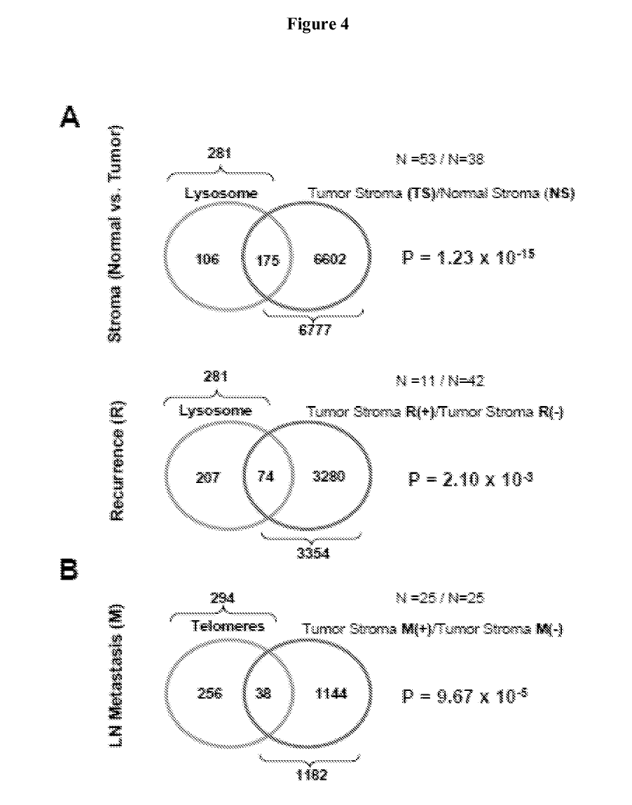

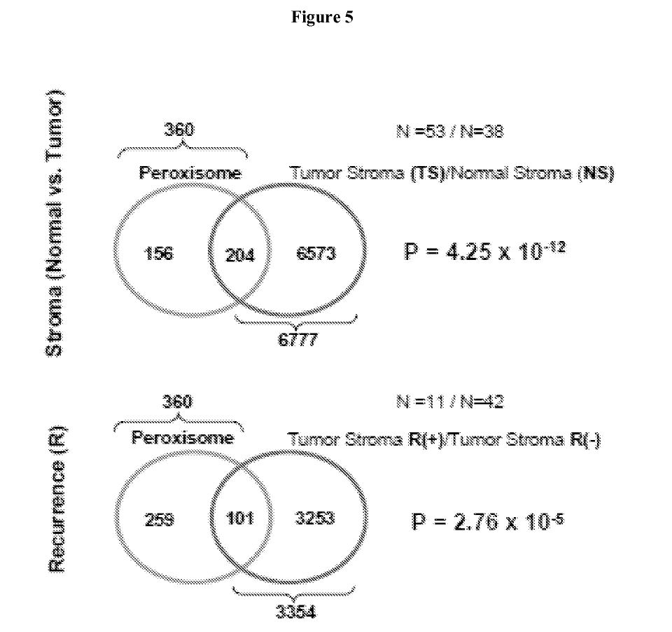

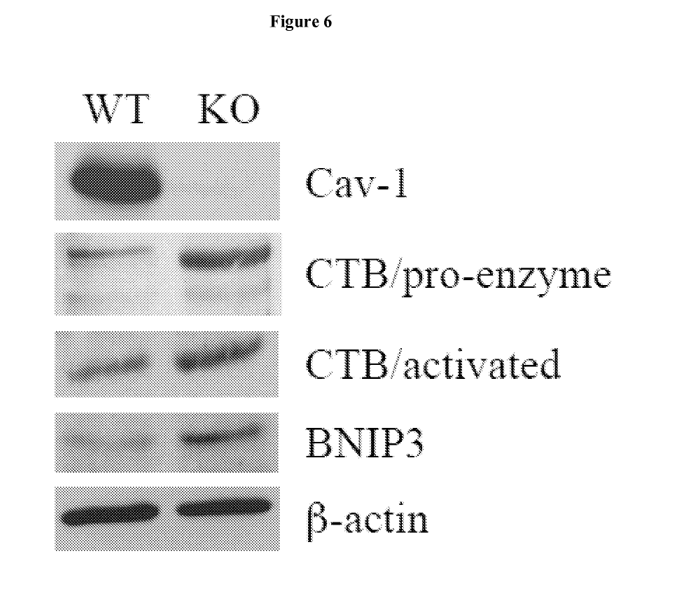

To independently assess the statistical association of autophagy, lysosomes, peroxisomes, and telomere-related gene transcripts with the human tumor stroma of breast cancer patients, the more comprehensive gene ontology lists were used to intersect with the tumor stroma, recurrence stroma, and metastasis stroma gene lists. The results of this more detailed analysis are presented in FIGS. 3, 4, and 5, and are represented as Venn diagrams. More specifically, FIG. 3 shows that autophagy-related genes are significantly associated with tumor stroma, recurrence-stroma, and metastasis-stroma. Similarly, lysosome-related genes were significantly associated with tumor stroma, and recurrencestroma, while telomere-related genes were only associated with metastasis-stroma FIG. 4, panels A and B). Finally, peroxisome-related genes were significantly associated with both tumor stroma and recurrence-stroma (FIG. 5). Thus, all of these inter-related biological processes (oxidative stress, autophagy/lysosomal degradation, and telomere-maintenance) may play a significant pathogenic role in generating an activated lethal tumor stroma.

### Example 4

### ADMA and Ketone Metabolism in Cav-1 (−/−) Stromal Cells and Human Tumor Stroma

Here, ADMA and 3-hydroxybutyrate (BHB) were identified as the two major metabolites, which increased in Cav-1 (−/−) null mammary fat pads and lung tissue, along with pyruvate to a lesser extent. These 2 metabolites (ADMA and BHB) are reflective of oxidative stress and mitochondrial dysfunction in Cav-1 (−/−) stromal cells.

To further validate these observations, transcriptional profiling data for the expression of the relevant enzymes that are involved in ADMA and ketone metabolism were analyzed. Both transcriptional profiles from Cav-1 (−/−) null stromal cells and human breast cancer tumor stroma were analyzed in parallel and are presented in Table 9, FIG. 29. For this purpose, the mRNA expression of the genes involved in ADMA production (PRMT gene family members) and degradation (DDAH1/2), as well as nitric oxide (NO) related genes, as ADMA drives NOS uncoupling and the production of superoxide, instead of NO 19 was analyzed. Interestingly, using this approach, the genes involved in both ADMA production (PRMT2/7/8) and degradation (DDAH1/2), as well as nitric oxide production (NOS1/2/3 or NOS trafficking), are all transcriptionally over-expressed. both in human tumor stroma and in Cav-1 (−/−) stromal cells.

Next, ketone metabolism was assessed (Table 9, FIG. 29). For this purpose the transcriptional profiles of the genes associated with both ketone production (ACYL, HMGCS1/2, HMGCL, HMGCLL1, and BDH1/2) and ketone re-utilization (ACAT1/2 and) OXCT1/2) were, analyzed. Interestingly, only the genes associated with ketone production, but not ketone re-utilization were associated with human tumor stroma. This is exactly as would be predicted, as the epithelial cancer cells should express the genes associated with ketone re-utilization, so that they can re-use 3-hydroxybutyrate as an energy source for mitochondrial oxidative metabolism. Also, many of the stromal genes involved in ketone production are specifically associated with tumor recurrence (ACLY, HMGCS2, HMGCLL1, and BDH1) and/or metastasis (BDH2). Many of these ketone production genes are also transcriptionally over-expressed in Cav-1 (−/−) stromal cells, consistent with our current metabolic analysis.

### Example 5

### Micro-RNA (miR) Profiling Provides New Mechanistic Insight into how Loss of Stromal Cav-1 Drives Oxidative Stress, Autophagy, and Mitochondrial Dysfunction

Since miRs have recently taken center stage in the molecular analysis of tumor progression and metastasis, Cav-1 (−/−) stromal cells were subjected to miR transcriptional profiling. Using this approach, a select subset of miRs that could explain the oxidative and catabolic phenotypes we observe in metabolically in Cav-1 (−/−) mammary fat pads could be identified. Table 10 shows that only a select number of miRs were transcriptionally upregulated in Cav-1 (−/−) stromal cells. For this analysis, a cut-off of 1.5-fold increased (KO/WT) was chosen. P-values are as shown. Note that top 2 miRs showed the most significant pvalues. Notably, miR-3) and miR-34c were increased 4.2-fold and nearly 3-fold, respectively.

A large body of evidence suggests that both miR-31 and miR-34c play prominent roles in both tumorigenesis and metastasis. miR-34c is normally induced under conditions of oxidative stress, DNA damage, and cellular senescence, consistent with our metabolic and mRNA transcriptional profiling data related to oxidative stress. miR-31, only the other hand, targets FIR (factor inhibiting HIF) 18. This, in turn, leads to the loss of FIH protein expression, driving HIF1-alpha transcriptional activation 18. Both hypoxia and HIF1-alpha activation itself are known to be sufficient to induce autophagy and mitophagy. Thus, loss of Cav-1 expression, driving miR-31 over-expression, and HIF1-alpha transcriptional activation, may be sufficient to explain our current findings related to oxidative stress, autophagy, and mitophagy. In accordance of role for Cav-1 in hypoxia regulation and HIF1-alpha transcriptional activation, an increase in miR-210 was observed, which is known to mediate many of the effects attributed to the hypoxia genetic transcriptional program 38. However, although miR-210 was increased nearly 2-fold, it did not reach statistical significance (p=0.07). miR-31 has recently been shown to be increased in the serum of patients with oral squamous cell cancers, and is dramatically reduced upon tumor resection, indicating that it can function as a marker for monitoring the course of cancer therapy. miR-31 is also upregulated in human colon cancers. Similarly, miR-210 is increased in the serum of pancreatic cancer patients.

### Example 6

### Over-Expression of Autophagy and Mitophagy Markers in Cav-1 (−/−) Null Mammary Fat Pads: Cathepsin B and BNIP3

To further validate that a loss of Cav-1 drives the onset of oxidative-stress induced autophagy, the expression of established autophagy markers, namely cathepsin B 42 and BNIP3, in Cav-1 (−/−) mammary fat pads was assessed. Cathepsin B is a well-known lysosomal cysteine protease that is up-regulated in the tumor stroma of human breast cancers, and its expression is also associated with tumor recurrence and metastasis (Table 7, FIG. 27). BNIP3 is a marker of autophagy which mediates the autophagic destruction of mitochondria, by a process called mitophagy. BNIP3 is also up-regulated by oxidative stress and/or hypoxia, and is under the direct transcriptional control of HIF1a. The stromal expression of BNIP3 is also associated with breast cancer tumor recurrence (Table 6, FIG. 26). Importantly, FIG. 18 directly shows that both cathepsin B (the pro-enzyme and activated form) and BNIP3 are significantly over-expressed in Cav-1 (−/−) null mammary fat pads, relative wild-type controls processed in parallel. Immunoblotting with Cav-1 and beta-actin are shown for comparison. These results are consistent with the idea that a loss of Cav-1 expression promotes the onset of autophagy in the tumor stromal compartment.

### Example 7

### Ketones Promote Tumor Growth, without any Increase in Angiogenesis

To evaluate the potential tumor promoting properties of the products of aerobic glycolysis (such as 3-hydroxy-butyrate and L-lacate), a xenograft model employing MDA-MB-231 breast cancer cells injected into the flanks of athymic nude mice was used. Tumor growth was assessed by measuring tumor volume, at 3-weeks post tumor cell injection. During this time period, mice were administered either PBS alone, or PBS containing 3-hydroxy-butyrate (500 mg/kg) or L-lactate (500 mg/kg), via daily intra-peritoneal (i.p.) injections.

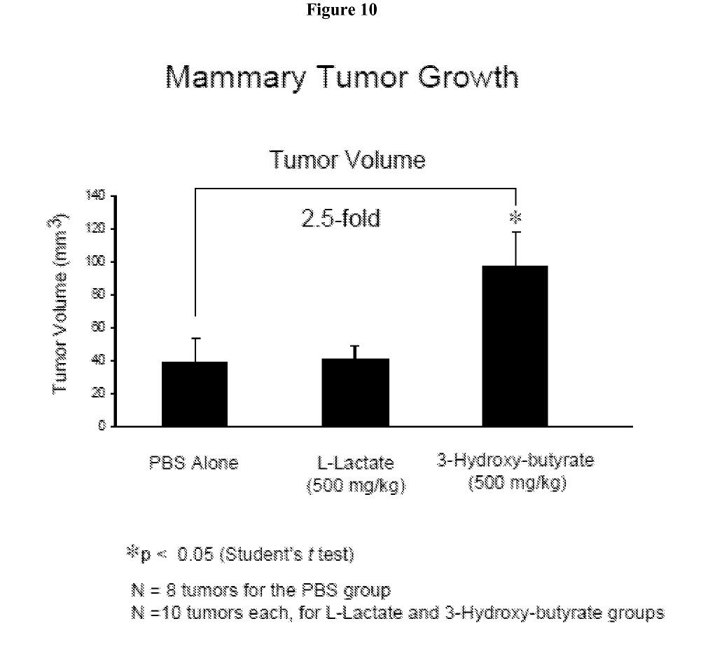

Interestingly, FIG. 10 shows that 3-hydroxy-butyrate is sufficient to promote an ˜2.5-fold increase in tumor growth, relative to the PBS-alone control. Under these conditions, L-lactate had no significant effect on tumor growth.

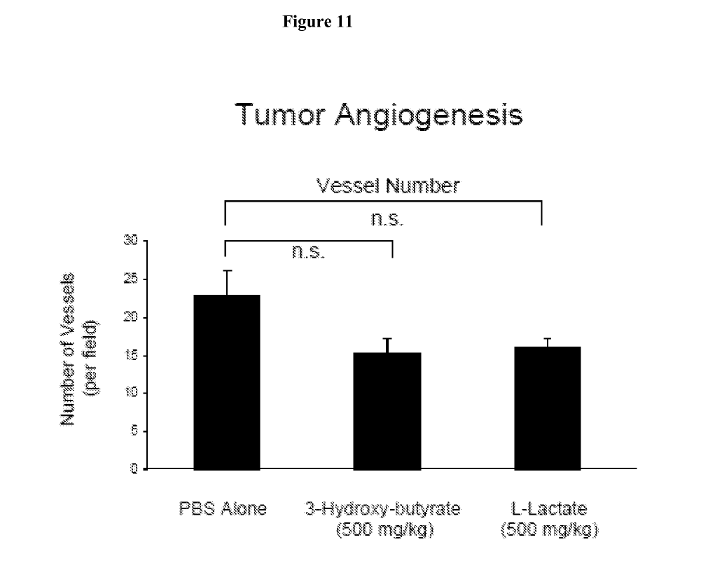

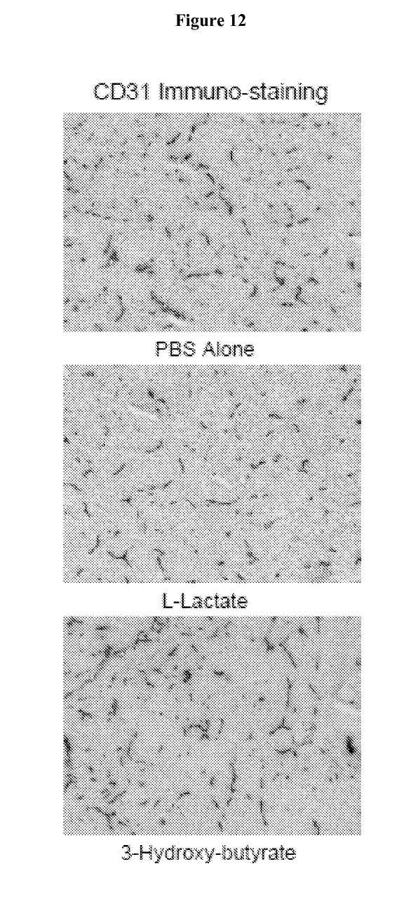

One mechanism that could account for the tumor-promoting properties of 3-hydroxy-butyrate is increased tumor angiogenesis. Thus, the status of tumor vascularity using antibodies directed against CD31 was evaluated. Interestingly, FIG. 11 shows that the vascular density (number of vessels per field) was not increased by the administration of either 3-hydroxy-butyrate or L-lactate. Thus, other mechanisms, such as the “Reverse Warburg Effect” may be operating to increase tumor growth.

### Example 8

### Ketones and Lactate Function as Chemo-Attractants, Stimulating Cancer Cell Migration

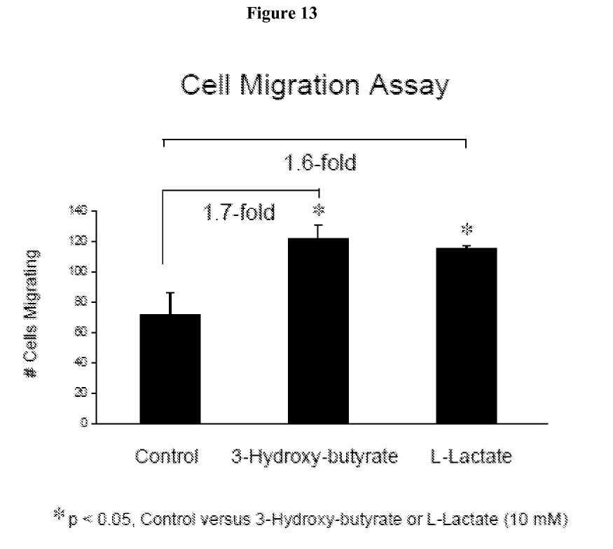

Next, whether 3-hydroxy-butyrate or L-lactate can function as chemo-attractants was assessed, using a modified “Boyden Chamber” assay, employing Transwell cell culture inserts. MDA-MB-231 cells were placed in the upper chambers, and 3-hydroxy-butyrate (10 mM) or L-lactate (10 mM) were introduced into the lower chambers. Interestingly, using this assay system, both 3-hydroxy-butyrate and L-lactate promoted cancer cell migration by nearly 2-fold (FIG. 13). Thus, the metabolic products of aerobic glycolysis can also function as chemo-attractants for cancer cells, probably via a form of nutrient sensing.

### Example 9

### Lactate Fuels Lung Metastasis

The effect of 3-hydroxy-butyrate and L-lactate on cancer cell metastasis were measured. For this purpose, a well-established lung colonization assay was used, where MDA-MB-231 cells are injected into the tail vein of athymic nude mice. After 7 weeks post-injection, the lungs were harvested and the metastases were visualized with India ink staining. In this method, the lung parenchyma stains black, while the tumor metastatic foci remain unstained, and appear white. For quantitation purposes. the number of metastases per lung lobe was scored.

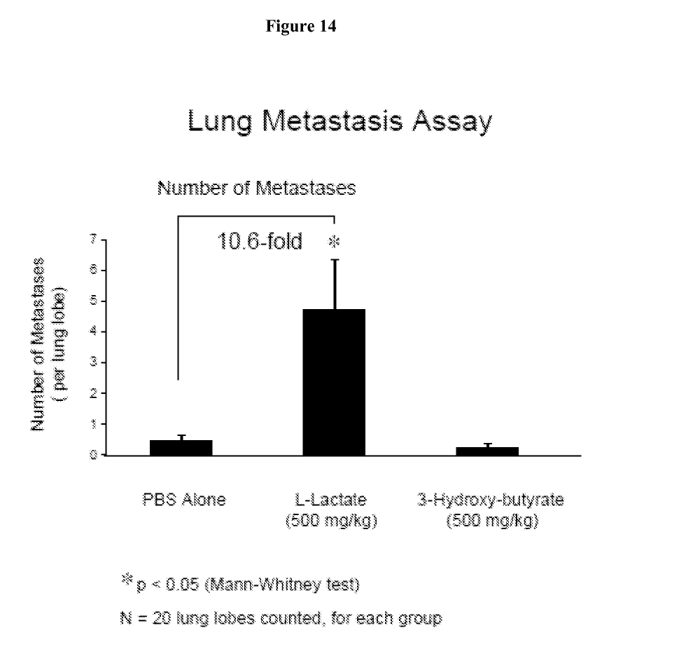

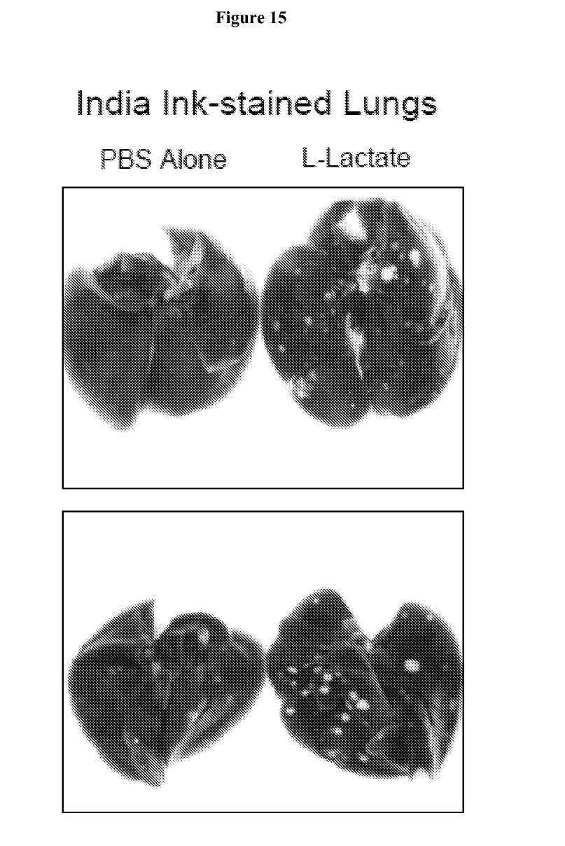

FIG. 14 shows that relative to PBS-alone, the administration of L-lacate stimulated the formation of metastatic foci by ˜10-fold. Under these conditions, 3-hydroxy-butryate had no effect on metastasis formation. Representative examples of lung metastasis in PBS-alone controls and L-lactate-treated animals are shown in FIG. 15. Note that the metastatic foci formed in L-lactate treated animals are more numerous, and are also larger in size.

Thus, 3-hydroxy-butryate fuels tumor growth, while L-lactate stimulates lung metastasis. As such, the “tumor-promoting” effects of 3-hydroxy-butyrate and L-lactate are remarkably specific to a given phase of tumor progression.

### Example 10

### Human Breast Cancer Epithelial Cells In Vivo Show the Induction of Mitochondrial Oxidative Phosphorylation, Relative to Adjacent Tumor Stromal Cells

To further validate the model that human breast cancer cells show a shift towards mitochondrial oxidative metabolism, raw transcriptional profiling data obtained by laser-capture micro-dissection of human breast cancer samples was assessed. In this data set, breast cancer epithelial cells and adjacent tumor stroma were isolated from the same tumors, allowing their direct comparison by transcriptional profiling.

For this purpose, we generated a list of genes that were transcriptionally up-regulated in human breast cancer epithelial cells, relative to the adjacent stromal cells. Then, this list of genes was intersected with existing databases to determine the cellular processes that were up-regulated in epithelial cancer cells, relative to adjacent stromal cells. Interestingly, using this approach, shows that numerous gene sets associated with oxidative mitochondrial metabolism are indeed increased or “enriched” in human breast cancer cells, relative to adjacent stromal cells (Table 1, FIG. 21). Conversely, this means that oxidative mitochondrial metabolism in down-regulated in the tumor stromal compartment, relative to the cancer cells, consistent with the “Reverse Warburg Effect”.

Moreover, the genes that were up-regulated in epithelial cancer cells were also down-regulated in response to starvation, hypoxia, and Alzheimer's disease/aging (associated with oxidative stress) (Table 1, FIG. 21). This is a strong indication these epithelial cancer cells are not experiencing starvation, hypoxia, or oxidative stress, as they are presumably being “fed” by glycolysis in adjacent stromal cells.

Interestingly, the number one gene up-regulated in cancer cells, relative to stromal cells, is a subunit of the mitochondrial enzyme isocitrate dehydrogenase (IDH3B; ˜7.5-fold increased; p=3.2×10-10) which catalyzes the oxidative de-carboxylation of isocitrate to 2-oxoglutarate, in the TCA cycle. During hypoxia, 2-oxoglutarate would accumulate, leading to HIF-stabilization via inhibition of the prolyl-hydroxylases. However, this appears not to be the case in the epithelial cancer cells, as the genes that they up-regulate are down-regulated in response to hypoxia and/or HIF1-alpha activation (Table 1, FIG. 21). This is a further indication that the epithelial cancer cells are indeed using oxidative mitochondrial metabolism.

These results provide independent clinically-relevant evidence that human epithelial breast cancer cells in vivo use oxidative mitochondrial metabolism in patients.

While the invention has been described in detail and with reference to specific examples thereof, it will be apparent to one skilled in the art that various changes and modifications can be made therein without departing from the spirit and scope thereof.

1. A method for making a prognosis of disease course in a human neoplastic disease patient, the method comprising the steps of:
(a) obtaining a sample from the patient, said sample comprising a tumor tissue, a body fluid or stromal cells adjacent to a neoplasm, or stromal cells within a neoplasm and/or a total tumor extract:
(b) determining the level of a biomarker selected from the group consisting of ACLY, HMGCS1, HMGCS2, HMGCL, HMGCLL1, BDH1, BDH2, BNIP3, BNIP3L, miR-3 1, miR-34c, ACAT1, ACAT2, OXCT1, OXCT2, ADMA, 3-hydroxybutyrate, and combinations thereof, in the sample;
wherein said prognosis is made when the level of biomarker in sample is higher than the level of biomarker in a control.

2. The method for making a prognosis of claim 1, wherein the biomarker is selected from the group consisting of ACLY, HMGCS1, HMGCS2, HMGCL, HMGCLL1, BDH1, BDH2, BNIP3, BNIP3L, and combinations thereof, in the sample.

3. The method for making a prognosis of claim 1, wherein making the prognosis of disease course comprises determining the likelihood that a carcinoma is of a grade likely to become an invasive carcinoma.

4. The method for making a prognosis of claim 3, wherein the biomarker is selected from the group consisting of ACLY, HMGCS1, HMGCS2, HMGCL, HMGCLL1, BDH1, BDH2, BNIP3, BNIP3L, and combinations thereof, in the sample.

5. The method for making a prognosis of claim 3, further comprising correlating the amount of labeling signal in the test sample with a control, wherein the carcinoma is of a grade likely to become invasive when the level of biomarker in the sample is higher than the level of biomarker in a control.

6. The method of claim 3, wherein the carcinoma is a carcinoma of the breast.

7. The method of claim 3, wherein the carcinoma is selected from the group consisting of carcinoma of the breast, skin, kidney, parotid gland, lung, bladder and prostate.

8. The method of claim 3, wherein the detection reagent is labeled antibody capable of binding to human ACLY, HMGCS1, HMGCS2, HMGCL, HMGCLL1, BDH1, BDH2, BNIP3, and/or BNIP3L.

9. The method of claim 3, wherein the detection reagent is labeled nucleic acid capable of binding to human ACLY, HMGCS1, HMGCS2, HMGCL, HMGCLL1, BDH1, BDH2, BNIP3, and/or BNIP3L.

10. The method of claim 3, wherein the amount of labeling signal is measured by a technique selected from the group consisting of emulsion autoradiography, phosphorimaging, light microscopy, confocal microscopy, multi-photon microscopy, and fluorescence microscopy.

11. The method of claim 3, wherein the amount of labeling signal is measured by autoradiography and a higher signal intensity in a test sample compared to a control prepared using the same steps as the test sample is used to diagnose a high grade carcinoma possessing a high probability the carcinoma will progress to an invasive carcinoma.

12. The method of claim 3, wherein the amount of labeling signal is measured by a technique selected from the group consisting of is an assay selected from the group consisting of RT-PCR, QRT-PCR, rolling circle amplification and nucleic acid sequenced-based amplification assays.

13. The method for making a prognosis of claim 1, wherein the human neoplastic disease patient has a neoplasm selected from the group consisting of breast, skin, kidney, lung, pancreas, rectum and colon, prostate, bladder, epithelial, non-epithelial, lymphomas, sarcomas, melanomas, and the like.

14. The method for making a prognosis of claim 1, wherein the human neoplastic disease patient has a breast neoplasm subtype selected from the group consisting of ER(+), PR(+), HER2(+), triple-negative (ER(−)/PR(−)/HER2(−)), ER(−), PR(−), all tumor and nodal stages, and all tumor grades.

15. The method for making a prognosis of claim 1, wherein the level of biomarker is determined by immunohistochemical staining.

16. The method for making a prognosis of claim 1, wherein the level of biomarker is determined by an assay selected from the group consisting of RT-PCR, QRT-PCR, rolling circle amplification and nucleic acid sequenced-based amplification assays.

17. The method of claim 1, wherein the level of biomarker is determined by enzymatic assay.

18. The method for making a prognosis of claim 1, wherein the prognosis of disease course includes a risk for metastasis, recurrence and relapse of neoplastic disease.

19. The method for making a prognosis of claim 1, wherein increase of biomarker predicts early disease recurrence, metastasis, survival, and tamoxifen-resistance at diagnosis.

20. The method for making a prognosis of claim 1, wherein INCREASE of biomarker in the sample predicts the prognosis of lymph-node positive (LN(+)) patients.

21. The method for making a prognosis of claim 1, wherein INCREASE of biomarker in the sample is associated with a poor prognosis.

22. The method for making a prognosis of claim 1, wherein the up-regulation or presence of biomarker in the sample is associated with a bad prognosis.

23. The method for making a prognosis of claim 1, wherein the neoplasm is a pre-malignant lesions selected from the group consisting of ductal carcinoma in situ (DCIS) of the breast and myelodysplastic syndrome of the bone marrow.

24. The method for making a prognosis of claim 1, wherein the prognosis of disease course includes staging malignant disease in a human neoplastic disease patient.

25. The method for making a prognosis of claim 1, wherein INCREASE of biomarker in the sample is a surrogate marker for stromal RB tumor suppressor functional inactivation by RB hyper-phosphorylation.

26. The method for making a prognosis of claim 1, wherein the body fluid is selected from the group consisting of plasma, serum, blood, lymphatic fluid, cerebrospinal fluid, synovial fluid, urine, saliva, mucous, phlegm, sputum, and combinations thereof.

27. The method for making a prognosis of claim 1, wherein the sample comprises DCIS breast tissue and stromal cells adjacent to a neoplasm, stromal cells within a neoplasm, and/or a total tumor extract from a human neoplastic disease patient.

28. The method for making a prognosis of claim 27, wherein the sample comprises ductal carcinoma in situ (DCIS) pre-invasive cancerous breast tissue, wherein the level of the biomarker in the stromal cells of the sample is compared with the level of biomarker with the level of biomarker in a control, and wherein the course of disease progression is made when the level of biomarker in the stromal cells of the sample is higher than the level of biomarker in the control.
